(19) United States
(12) Patent Application Publication
(10) Pub. No.: US 2022/0195065 A1
(43) Pub. Date: Jun. 23, 2022

(54) PHARMACEUTICAL FORMULATIONS AND DOSAGE REGIMENS FOR MULTI-SPECIFIC BINDING PROTEINS THAT BIND HER2, NKG2D, AND CD16 FOR CANCER TREATMENT

(71) Applicant: Dragonfly Therapeutics, Inc., Waltham, MA (US)

(72) Inventors: Gregory P. Chang, Medford, MA (US); Ann F. Cheung, Lincoln, MA (US); Jean-Marie Cuillerot, Somerville, MA (US); Daniel Fallon, Winchester, MA (US); Asya Grinberg, Lexington, MA (US); William Haney, Wayland, MA (US); Christopher Ryan Morgan, Southborough, MA (US); Michael C. Naill, Stow, MA (US); Steven O'Neil, Wayland, MA (US); Nicolai Wagtmann, Concord, MA (US); Ronnie Wei, Weston, MA (US)

(21) Appl. No.: 17/682,367

(22) Filed: Feb. 28, 2022

Related U.S. Application Data
(63) Continuation of application No. PCT/US2020/048500, filed on Aug. 28, 2020.
(60) Provisional application No. 62/916,935, filed on Oct. 18, 2019.
(60) Provisional application No. 62/895,320, filed on Sep. 3, 2019.
(60) Provisional application No. 62/894,047, filed on Aug. 30, 2019.

(51) Int. Cl. C07K 16/30; C07K 14/725; C07K 16/28; C07K 16/32

(52) U.S. Cl. CPC C07K 16/30; C07K 2317/94; C07K 16/283; C07K 16/2851; C07K 16/2878; C07K 16/32; C07K 2317/21; C07K 2317/24; C07K 2317/31; C07K 2317/524; C07K 2317/526; C07K 2317/55; C07K 2317/622; C07K 2317/64; C07K 14/7051

(57) ABSTRACT

This disclosure relates to pharmaceutical formulations for multi-specific binding proteins having an epidermal growth factor receptor 2 (ErbB2 or HER2)-binding scFv, an NKG2D-binding Fab, and an antibody Fc domain. Also provided are dosage regimens for such multi-specific binding proteins and pharmaceutical formulations for use in treating cancer, such as locally advanced or metastatic solid tumor.

### CROSS-REFERENCE TO RELATED APPLICATIONS

This application claims the benefit of and priority to U.S. Provisional Patent Application No. 62/894,047, filed on Aug. 30, 2019; U.S. Provisional Patent Application No. 62/895,320, filed on Sep. 3, 2019; and U.S. Provisional Patent Application No. 62/916,935, filed on Oct. 18, 2019, the disclosure of each of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Aug. 27, 2020, is named DFY-078WO_SL.txt and is 194,972 bytes in size.

### FIELD OF THE DISCLOSURE

The present disclosure relates generally to pharmaceutical formulations for multi-specific binding proteins having an epidermal growth factor receptor 2 (ErbB2 or HER2)-binding single-chain variable fragment (scFv), an NKG2D-binding Fab, and an antibody Fc domain, and dosage regimens for such multi-specific binding proteins and pharmaceutical formulations for use in treating cancer, such as locally advanced or metastatic solid tumor.

### BACKGROUND

Cancer continues to be a significant health problem despite the substantial research efforts and scientific advances reported in the literature for treating this disease. Cancer immunotherapies are being developed to facilitate destruction of cancer cells using the patient's own immune system. The immune cells activated by cancer immunotherapies include T cells and natural killer (NK) cells. For example, bispecific T-cell engagers are designed to direct T cells against tumor cells, thereby rendering cytotoxicity against the tumor cells. Bispecific antibodies that bind NK cells and a tumor-associated antigen (TAA) have also been created for cancer treatment (see, e.g., WO 2016/134371).

HER2 is a transmembrane glycoprotein in the epidermal growth factor receptor family It is a receptor tyrosine kinase and regulates cell survival, proliferation, and growth. HER2 plays an important role in human malignancies. The ERBB2 gene is amplified or overexpressed in approximately 30% of human breast cancers. Patients with HER2-overexpressing breast cancer have substantially lower overall survival rates and shorter disease-free intervals than patients whose cancer does not overexpress HER2. Moreover, overexpression of HER2 leads to increased breast cancer metastasis. Over-expression of HER2 is also known to occur in many other cancer types, including ovarian, esophageal, bladder and gastric cancer, salivary duct carcinoma, adenocarcinoma of the lung, and aggressive forms of uterine cancer, such as uterine serous endometrial carcinoma.

Multi-specific binding proteins that bind HER2 and one or more immune cell surface proteins have been studied. For example, WO 2018/152518 describes multi-specific binding proteins that bind HER2, NKG2D, and CD16. The present disclosure adds to these developments and provides clinical methods, including dosage regimens, to treat patients with specific HER2-targeting cancer immunotherapies with desired safety and efficacy. Furthermore, the present disclosure adds to the earlier developments in the field by providing formulations comprising such cancer immunotherapies that are sufficiently stable and suitable for administration to patients.

### SUMMARY OF THE DISCLOSURE

The present disclosure provides pharmaceutical formulations comprising a multi-specific binding protein having a HER2-binding scFv, an NKG2D-binding Fab, and an antibody Fc domain, the ingredients in the formulation optimized for stability of the multi-specific binding proteins. Also provided are dosage regimens for using the multi-specific binding proteins and pharmaceutical formulations in treating cancer, such as locally advanced or metastatic solid tumor.

Accordingly, in one aspect, the present disclosure provides a pharmaceutical formulation at a pH of 5.5 to 6.5 that includes histidine, a polysorbate, a sugar or sugar alcohol, and a multi-specific binding protein that includes an antibody Fc domain, a Fab that binds NKG2D, and a single-chain variable fragment (scFv) that binds HER2.

In certain embodiments, the concentration of histidine in the pharmaceutical formulation is 10 to 25 mM. In certain embodiments, the concentration of histidine in the pharmaceutical formulation is about 20 mM.

In certain embodiments, the sugar or sugar alcohol is a disaccharide. In certain embodiments, the disaccharide is sucrose. In certain embodiments, the sugar or sugar alcohol is a sugar alcohol derived from a monosaccharide. In certain embodiments, the sugar alcohol derived from a monosaccharide is sorbitol. In certain embodiments, the concentration of the sugar or sugar alcohol in the pharmaceutical formulation is 200 to 300 mM. In certain embodiments, the concentration of sugar or sugar alcohol in the pharmaceutical formulation is about 250 mM.

In certain embodiments, the polysorbate is polysorbate 80. In certain embodiments, the concentration of polysorbate 80 in the pharmaceutical formulation is 0.005% to 0.05%. In certain embodiments, the concentration of polysorbate 80 in the pharmaceutical formulation is about 0.01%.

In certain embodiments, the concentration of NaCl, if any, is about 10 mM or lower in the pharmaceutical formulation. In certain embodiments, the concentration of NaCl, if any, is about 1 mM or lower in the pharmaceutical formulation.

In certain embodiments, the pH of the pharmaceutical formulation is 5.8 to 6.2. In certain embodiments, the pH of the pharmaceutical formulation is 5.95 to 6.05.

In certain embodiments, the concentration of the multi-specific binding protein in the pharmaceutical formulation is about 10 to about 20 mg/mL.

In certain embodiments, more than 94% of the multi-specific binding protein has native conformation, as determined by size-exclusion chromatography, after incubation at 50° C. for 3 weeks. In certain embodiments, less than 4% of the multi-specific binding protein forms a high molecular weight complex, as determined by size-exclusion chromatography, after incubation at 50° C. for 3 weeks.

In another aspect, the present disclosure provides use of a pharmaceutical formulation disclosed herein in treating cancer. In certain embodiments, the pharmaceutical formulation is diluted with 0.9% NaCl solution prior to the use.

In another aspect, the present disclosure provides a method for treating cancer, the method comprising administering to a subject in need thereof a multi-specific binding protein in an initial four-week treatment cycle on Day 1, Day 8, and Day 15, wherein the multi-specific binding protein comprises: (a) a Fab that binds NKG2D; (b) an scFv that binds HER2; and (c) an antibody Fc domain.

In certain embodiments, the method further comprises administering to the subject, after the initial treatment cycle, the multi-specific binding protein in one or more subsequent four-week treatment cycles, wherein the multi-specific binding protein is administered on Day 1 and Day 15 in each subsequent treatment cycle. In certain embodiments, each of the doses comprises the multi-specific binding protein at an amount selected from the group consisting of 5.2×10⁻⁵ mg/kg, 1.6×10⁻⁴ mg/kg, 5.2×10⁻⁴ mg/kg, 1.6×10⁻³ mg/kg, 5.2×10⁻³ mg/kg, 1.6×10⁻² mg/kg, 5.2×10⁻² mg/kg, 1.6×10⁻¹ mg/kg, 0.52 mg/kg, 1 mg/kg, 1.6 mg/kg, 5.2 mg/kg, 10 mg/kg, 20 mg/kg, and 50 mg/kg. In certain embodiments, the multi-specific binding protein is administered by intravenous infusion.

In certain embodiments, the multi-specific binding protein is used as a monotherapy.

In certain embodiments, the method further comprises administering to the subject an anti-PD-1 antibody. In certain embodiments, the anti-PD-1 antibody is pembrolizumab. In certain embodiments, 200 mg of pembrolizumab is administered on Day 1 of the initial treatment cycle. In certain embodiments, if the subject receives one or more subsequent treatment cycles, 200 mg of pembrolizumab is administered once every three weeks in the subsequent treatment cycles.

In certain embodiments, the cancer is HER2-positive as determined by immunohistochemistry. In certain embodiments, the HER2 level in the cancer is scored at least 1+ as determined by immunohistochemistry. In certain embodiments, the HER2 level in the cancer is scored 2+ or 3+. In certain embodiments, the HER2 level in the cancer is scored 3+.

In certain embodiments, the cancer has amplification of the ERBB2 gene. In certain embodiments, the ERBB2 gene amplification is determined by fluorescent in situ hybridization. In certain embodiments, the ERBB2 gene amplification is determined by DNA sequencing.

In certain embodiments, the cancer is a solid tumor. In certain embodiments, the cancer is a locally advanced or metastatic solid tumor. In certain embodiments, the cancer is selected from the group consisting of gastric cancer, colorectal cancer, non-small cell lung cancer (NSCLC), head and neck cancer, biliary tract cancer, glioblastoma, sarcoma, uterine cancer, cervical cancer, ovarian cancer, esophageal cancer, squamous carcinoma of the skin, prostate cancer, carcinoma of the salivary gland, breast cancer, pancreatic cancer, and gallbladder cancer. In certain embodiments, the cancer is urothelial bladder cancer or metastatic breast cancer.

The following features can be incorporated into any of the embodiments recited above:

In certain embodiments, the Fab comprises a heavy chain variable domain and a light chain variable domain, wherein (a) the heavy chain variable domain comprises complementarity-determining region 1 (CDR1), complementarity-determining region 2 (CDR2), and complementarity-determining region 3 (CDR3) sequences represented by the amino acid sequences of SEQ ID NOs: 168, 96, and 188, respectively; and (b) the light chain variable domain comprises CDR1, CDR2, and CDR3 sequences represented by the amino acid sequences of SEQ ID NOs: 99, 100, and 101, respectively.

In certain embodiments, (a) the heavy chain variable domain comprises CDR1, CDR2, and CDR3 sequences represented by the amino acid sequences of SEQ ID NOs: 168, 96, and 169, respectively; and (b) the light chain variable domain comprises CDR1, CDR2, and CDR3 sequences represented by the amino acid sequences of SEQ ID NOs: 99, 100, and 101, respectively. In certain embodiments, the heavy chain variable domain of the Fab comprises an amino acid sequence at least 90% identical to SEQ ID NO:94, and the light chain variable domain comprises an amino acid sequence at least 90% identical to SEQ ID NO:98. In certain embodiments, the heavy chain variable domain of the Fab comprises the amino acid sequence of SEQ ID NO:94, and the light chain variable domain comprises the amino acid sequence of SEQ ID NO:98.

In certain embodiments, the scFv comprises a heavy chain variable domain and a light chain variable domain, wherein (a) the heavy chain variable domain comprises CDR1, CDR2, and CDR3 sequences represented by the amino acid sequences of SEQ ID NOs: 115, 116, and 117, respectively; and (b) the light chain variable domain comprises CDR1, CDR2, and CDR3 sequences represented by the amino acid sequences of SEQ ID NOs: 119, 120, and 121, respectively. In certain embodiments, the heavy chain variable domain of the scFv comprises an amino acid sequence at least 90% identical to SEQ ID NO:195, and the light chain variable domain of the scFv comprises an amino acid sequence at least 90% identical to SEQ ID NO:196. In certain embodiments, the heavy chain variable domain of the scFv comprises the amino acid sequence of SEQ ID NO:195, and the light chain variable domain of the scFv comprises the amino acid sequence of SEQ ID NO:196.

In certain embodiments, the light chain variable domain of the scFv is linked to the heavy chain variable domain of the scFv via a flexible linker. In certain embodiments, the flexible linker comprises the amino acid sequence of SEQ ID NO:143. In certain embodiments, the flexible linker consists of the amino acid sequence of SEQ ID NO:143. In certain embodiments, the light chain variable domain of the scFv is positioned to the N-terminus of the heavy chain variable domain of the scFv.

In certain embodiments, the heavy chain variable domain of the scFv forms a disulfide bridge with the light chain variable domain of the scFv. In certain embodiments, the disulfide bridge is formed between C44 of the heavy chain variable domain and C100 of the light chain variable domain.

In certain embodiments, the scFv comprises the amino acid sequence of SEQ ID NO:139.

In certain embodiments, the antibody Fc domain comprises a first antibody Fc sequence linked to the Fab and a second antibody Fc sequence linked to the scFv. In certain embodiments, the first antibody Fc sequence is linked to the heavy chain portion of the Fab. In certain embodiments, the scFv is linked to the second antibody Fc sequence via a hinge comprising Ala-Ser.

In certain embodiments, the first and second antibody Fc sequences each comprise a hinge and a CH2 domain of a human IgG1 antibody. In certain embodiments, the first and second antibody Fc sequences each comprise an amino acid sequence at least 90% identical to amino acids 234-332 of a wild-type human IgG1 antibody.

In certain embodiments, the first and second antibody Fc sequences comprise different mutations promoting heterodimerization. In certain embodiments, the first antibody Fc sequence is a human IgG1 Fc sequence comprising K360E and K409W substitutions. In certain embodiments, the second antibody Fc sequence is a human IgG1 Fc sequence comprising Q347R, D399V, and F405T substitutions.

In certain embodiments, the multi-specific binding protein comprises (a) a first polypeptide comprising the amino acid sequence of SEQ ID NO:141; (b) a second polypeptide comprising the amino acid sequence of SEQ ID NO:140; and (c) a third polypeptide comprising the amino acid sequence of SEQ ID NO:142.

Other embodiments and details of the disclosure are presented herein below.

### BRIEF DESCRIPTION OF THE DRAWINGS

FIG. 1 illustrates a trispecific antibody (TriNKET) that contains a HER2-binding scFv, a NKG2D-targeting Fab, and a heterodimerized antibody Fc domain that binds CD16 (the “F3′-TriNKET” format). In an exemplary embodiment, the Fc domain linked to the Fab fragment comprises mutations K360E and K409W, and the Fc domain linked to the scFv comprises matching mutations Q347R, D399V, and F405T for forming an Fc heterodimer (shown as a triangular lock-and-key format in the Fc domains in FIG. 1). In another exemplary embodiment, the Fc domain linked to the Fab fragment comprises the mutations of Q347R, D399V, and F405T, and the Fc domain linked to the scFv comprises matching mutations K360E and K409W for forming a heterodimer.

FIG. 2A is an interaction plot for average size as measured by Dynamic Light Scattering (DLS) after a 3-week incubation at 50° C. FIG. 2B is an interaction plot for average size as measured by DLS after a 3-week incubation at 2-8° C.

FIG. 3A is an interaction plot for monomer size as measured by DLS after a 3-week incubation at 50° C. FIG. 3B is an interaction plot for monomer size as measured by DLS after a 3-week incubation at 2-8° C.

FIG. 4A is an interaction plot for % main species determined by Size Exclusion Chromatography (SEC) for a 3-week incubation at 50° C. FIG. 4B is an interaction plot for % main species determined by SEC for a 3-week incubation at 2-8° C.

FIG. 5A is an interaction plot for percent High Molecular Weight (% HMW) species determined by SEC for a 3-week incubation at 50° C. FIG. 5B is an interaction plot for % HMW species determined by SEC for a 3-week incubation at 2-8° C.

FIG. 6A is an interaction plot for percent Low Molecular Weight (% LMW) species determined by SEC for a 3-week incubation at 50° C. FIG. 6B is an interaction plot for % LMW species determined by SEC for a 3-week incubation at 2-8° C. at pH 6.0.

FIG. 7A is an interaction plot for % acidic species determined by Imaged Capillary Isoelectric Focusing (icIEF) for a 3-week incubation at 50° C. FIG. 7B is an interaction plot for % basic species for sucrose only as determined by icIEF for a 3-week incubation 4-8° C.

FIG. 8A is an interaction plot for % main species determined by icIEF for a 3-week incubation at 50° C. FIGS. 8B-8D are interaction plots for % main species in the sucrose only formulation, determined by icIEF, for a 3-week incubation at 4° C. at pH 5.5 (FIG. 8B), pH 6.0 (FIG. 8C), and pH 6.5 (FIG. 8D).

FIG. 9A is an interaction plot for % basic species determined by icIEF for a 3-week incubation at 50° C. FIG. 9B is an interaction plot for % basic species for sucrose only as determined by icIEF for a 3-week incubation 2-8° C.

FIG. 10A is an interaction plot for % purity determined by Capillary Electrophoresis (CE) for a 3-week incubation at 50° C. FIG. 10B is an interaction plot for % impurities determined by CE for a 3-week incubation at 50° C.

FIG. 11A is an interaction plot for % main species determined by Capillary Electrophoresis (Non-Reduced) (CE (NR)) for a 3-week incubation at 50° C. at pH 6.0. FIG. 11B is an interaction plot for %main species determined by CE (NR) for a 3-week incubation at 2-8° C. at pH 6.0.

FIG. 12A is an interaction plot for % HMW species determined by CE (NR) for a 3-week incubation at 50° C. FIG. 12B shows the interaction plot for % HMW species determined by CE (NR) for a 3-week incubation at 2-8° C.

FIG. 13A is an interaction plot for % LMW species for sucrose only as determined by CE (NR) for a 3-week incubation at 50° C. FIG. 13B is an interaction plot for % LMW species for sucrose only as determined by CE (NR) for a 3-week incubation at 2-8° C.

FIGS. 14A-14B is a schematic diagram of a clinical trial design. FIG. 14A describes the trial design for a dose escalation phase. FIG. 14B describes the trial design for an efficacy expansion cohorts phase. Abbreviations used in the figures include: DL=dose level; Combo PD-1=combination therapy with pembrolizumab; PK=pharmacokinetics; PD=pharmacodynamics; Her2 HIGH=high expression of HER2 of 3+, per immunohistochemistry; MBC HER2 2+/1+=metastatic breast cancer with medium/low expression of HER2 of 2+/1+, per immunohistochemistry; UBC 2L/3L=urothelial bladder cancer 2nd line-/3rd line treatment.

### DETAILED DESCRIPTION

### Definitions

To facilitate an understanding of the present invention, a number of terms and phrases are defined below.

The terms “a” and “an” as used herein mean “one or more” and include the plural unless the context is inappropriate.

As used herein, the terms “Fab” and “scFv” refer to two different forms of protein fragments that each include an antigen-binding site. The term “antigen-binding site” refers to the part of the immunoglobulin molecule that participates in antigen binding. In human antibodies, the antigen-binding site is formed by amino acid residues of the N-terminal variable (“V”) regions of the heavy (“H”) and light (“L”) chains, which are also called “VH” and “VL,” respectively. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface such as a Fab, or in a recombinant polypeptide such as an scFv, using a peptide linker to connect the heavy chain variable domain to the light chain variable domain in a single polypeptide. All the amino acid positions in heavy or light chain variable regions disclosed herein are numbered according to Kabat numbering.

The CDRs of an antigen-binding site can be determined by the methods described in Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared against each other. In certain embodiments, the term “CDR” is a CDR as defined by MacCallum et al., J. Mol. Biol. 262:732-745 (1996) and Martin A., Protein Sequence and Structure Analysis of Antibody Variable Domains, in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. For example, in certain embodiments, the heavy chain CDRs are defined according to MacCallum (supra), and the light CDRs are defined according to Kabat (supra). CDRH1, CDRH2 and CDRH3 denote the heavy chain CDRs, and CDRL1, CDRL2 and CDRL3 denote the light chain CDRs.

As used herein, the term “pharmaceutical formulation” refers to the combination of an active agent with a carrier, inert or active, making the composition especially suitable for diagnostic or therapeutic use in vivo or ex vivo.

As used herein, the terms “subject” and “patient” refer to an organism to be treated by the methods and compositions described herein. Such organisms preferably include, but are not limited to, mammals (e.g., murines, simians, equines, bovines, porcines, primates, canines, felines, and the like), and more preferably include humans.

The terms “treat,” “treating,” or “treatment,” and other grammatical equivalents as used in this disclosure, include alleviating, abating, ameliorating, or preventing a disease, condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder.

The term “about” refers to any minimal alteration in the concentration or amount of an agent that does not change the efficacy of the agent in preparation of a formulation and in treatment of a disease or disorder. In certain embodiments, the term “about” may include ±5%, ±10%, or ±15% of a specified numerical value or data point.

Ranges can be expressed in this disclosure as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed in this disclosure, and that each value is also disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### Multi-Specific Binding Proteins

The present disclosure provides pharmaceutical formulations comprising a multi-specific binding protein having a HER2-binding scFv, an NKG2D-binding Fab, and an antibody Fc domain, the ingredients in the formulation optimized for stability of the multi-specific binding proteins. Also provided are dosage regimens for using the multi-specific binding proteins and pharmaceutical formulations in treating cancer, such as a locally advanced or metastatic solid tumor. The multi-specific binding proteins are capable of binding HER2 on a cancer cell and NKG2D and CD16 on natural killer cells. Such binding brings the cancer cell into proximity with the natural killer cell, which facilitates direct and indirect destruction of the cancer cell by the natural killer cells.

The first component of the multi-specific binding proteins binds to NKG2D receptor-expressing cells, which can include but are not limited to NK cells, NKT cells, γδ T cells and CD8⁺ αβ T cells. Upon NKG2D binding, the multi-specific binding proteins may block natural ligands, such as ULBP6 and MICA, from binding to NKG2D and activating NK cells. The second component of the multi-specific binding proteins binds to HER2-expressing cells, which can include but are limited to breast, ovarian, esophageal, bladder and gastric cancer, salivary duct carcinoma, adenocarcinoma of the lung and aggressive forms of uterine cancer, such as uterine serous endometrial carcinoma. The third component of the multi-specific binding proteins is an antibody Fc domain, which binds to cells expressing CD16 such as NK cells, macrophages, neutrophils, eosinophils, mast cells, and follicular dendritic cells.

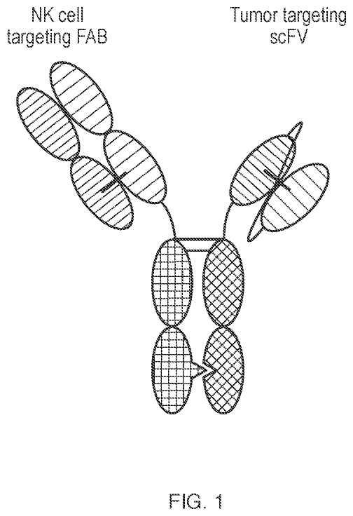

The multi-specific binding proteins described herein can take various formats. For example, one format involves a heterodimeric, multi-specific antibody including a first immunoglobulin heavy chain, a second immunoglobulin heavy chain and an immunoglobulin light chain (FIG. 1). The first immunoglobulin heavy chain includes a first antibody Fc sequence (hinge-CH2-CH3) fused via either a linker or an antibody hinge to the heavy chain portion of a Fab, which includes a heavy chain variable domain and a CH1 domain. The immunoglobulin light chain includes the light chain portion of a Fab, which includes a light chain variable domain and a light chain constant domain (CL), wherein the heavy chain and light chain portions of the Fab fragment pair and bind NKG2D. The second immunoglobulin heavy chain includes a second antibody Fc sequence (hinge-CH2-CH3) fused via either a linker or an antibody hinge to a single-chain variable fragment (scFv) composed of a heavy chain variable domain and a light chain variable domain which pair and bind HER2.

Individual components of the multi-specific binding proteins are described in more detail below.

### NKG2D-Binding Site

Upon binding to the NKG2D receptor and CD16 receptor on natural killer cells and a tumor-associated antigen on cancer cells, the multi-specific binding proteins can engage more than one kind of NK-activating receptor, and may block the binding of natural ligands to NKG2D. In certain embodiments, the proteins can agonize NK cells in humans. In some embodiments, the proteins can agonize NK cells in humans and in other species such as rodents and cynomolgus monkeys.

Table 1 lists peptide sequences of heavy chain variable domains and light chain variable domains that, in combination, can bind to NKG2D. In some embodiments, the heavy chain variable domain and the light chain variable domain are arranged in Fab format. These NKG2D binding domains can vary in their binding affinity to NKG2D, nevertheless, they all activate human NK cells. Unless indicated otherwise, the CDR sequences provided in Table 1 are determined under Kabat.

TABLE 1
Exemplary NKG2D-Binding Sites

| | Heavy chain variable domain amino | Light chain variable domain |
|---|---|---|
| Clones | acid sequence | amino acid sequence |
| ADI-27705 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | NSYPITFGGGTKVEIK |
| | (SEQ ID NO: 1) | (SEQ ID NO: 2) |
| | CDR1 (SEQ ID NO: 3) - | |
| | GSFSGYYWS | |
| | CDR2 (SEQ ID NO: 4) - | |
| | EIDHSGSTNYNPSLKS | |
| | CDR3 (SEQ ID NO: 5) - | |
| | ARARGPWSFDP | |
| ADI-27724 | QVQLQQWGAGLLKPSETLSLTCAV | EIVLTQSPGTLSLSPGERATLSCR |
| | YGGSFSGYYWSWIRQPPGKGLEWI | ASQSVSSSYLAWYQQKPGQAPR |
| | GEIDHSGSTNYNPSLKSRVTISVDTS | LLIYGASSRATGIPDRFSGSGSG |
| | KNQFSLKLSSVTAADTAVYYCARA | TDFTLTISRLEPEDFAVYYCQQY |
| | RGPWSFDPWGQGTLVTVSS | GSSPITFGGGTKVEIK |
| | (SEQ ID NO: 6) | (SEQ ID NO: 7) |
| ADI-27740 | QVQLQQWGAGLLKPSETLSLTCAV | DIQMTQSPSTLSASVGDRVTITC |
| (A40) | YGGSFSGYYWSWIRQPPGKGLEWI | RASQSIGSWLAWYQQKPGKAP |
| | GEIDHSGSTNYNPSLKSRVTISVDTS | KLLIYKASSLESGVPSRFSGSGS |
| | KNQFSLKLSSVTAADTAVYYCARA | GTEFTLTISSLQPDDFATYYCQQ |
| | RGPWSFDPWGQGTLVTVSS | YHSFYTFGGGTKVEIK |
| | (SEQ ID NO: 8) | (SEQ ID NO: 9) |
| ADI-27741 | QVQLQQWGAGLLKPSETLSLTCAV | DIQMTQSPSTLSASVGDRVTITC |
| | YGGSFSGYYWSWIRQPPGKGLEWI | RASQSIGSWLAWYQQKPGKAP |
| | GEIDHSGSTNYNPSLKSRVTISVDTS | KLLIYKASSLESGVPSRFSGSGS |
| | KNQFSLKLSSVTAADTAVYYCARA | GTEFTLTISSLQPDDFATYYCQQ |
| | RGPWSFDPWGQGTLVTVSS | SNSYYTFGGGTKVEIK |
| | (SEQ ID NO: 10) | (SEQ ID NO: 11) |
| ADI-27743 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | NSYPTFGGGTKVEIK |
| | (SEQ ID NO: 12) | (SEQ ID NO: 13) |
| ADI-28153 | QVQLQQWGAGLLKPSETLSLTCAV | ELQMTQSPSSLSASVGDRVTITC |
| | YGGSFSGYYWSWIRQPPGKGLEWI | RTSQSISSYLNWYQQKPGQPPK |
| | GEIDHSGSTNYNPSLKSRVTISVDTS | LLIYWASTRESGVPDRFSGSGSG |
| | KNQFSLKLSSVTAADTAVYYCARA | TDFTLTISSLQPEDSATYYCQQS |
| | RGPWGFDPWGQGTLVTVSS | YDIPYTFGQGTKLEIK |
| | (SEQ ID NO: 14) | (SEQ ID NO: 15) |
| ADI-28226 | QVQLQQWGAGLLKPSETLSLTCAV | DIQMTQSPSTLSASVGDRVTITC |
| (C26) | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | GSFPITFGGGTKVEIK |
| | (SEQ ID NO: 16) | (SEQ ID NO: 17) |
| ADI-28154 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TDFTLTISSLQPDDFATYYCQQS |
| | RGPWSFDPWGQGTLVTVSS | KEVPWTFGQGTKVEIK |
| | (SEQ ID NO: 18) | (SEQ ID NO: 19) |
| ADI-29399 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | NSFPTFGGGTKVEIK |
| | (SEQ ID NO: 20) | (SEQ ID NO: 21) |
| ADI-29401 | QVQLQQWGAGLLKPSETLSLTCAV | DIQMTQSPSTLSASVGDRVTITC |
| | YGGSFSGYYWSWIRQPPGKGLEWI | RASQSIGSWLAWYQQKPGKAP |
| | GEIDHSGSTNYNPSLKSRVTISVDTS | KLLIYKASSLESGVPSRFSGSGS |
| | KNQFSLKLSSVTAADTAVYYCARA | GTEFTLTISSLQPDDFATYYCQQ |
| | RGPWSFDPWGQGTLVTVSS | YDIYPTFGGGTKVEIK |
| | (SEQ ID NO: 22) | (SEQ ID NO: 23) |
| ADI-29403 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | DSYPTFGGGTKVEIK |
| | (SEQ ID NO: 24) | (SEQ ID NO: 25) |
| ADI-29405 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | GSFPTFGGGTKVEIK |
| | (SEQ ID NO: 26) | (SEQ ID NO: 27) |
| ADI-29407 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | QSFPTFGGGTKVEIK |
| | (SEQ ID NO: 28) | (SEQ ID NO: 29) |
| ADI-29419 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | SSFSTFGGGTKVEIK |
| | (SEQ ID NO: 30) | (SEQ ID NO: 31) |
| ADI-29421 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | ESYSTFGGGTKVEIK |
| | (SEQ ID NO: 32) | (SEQ ID NO: 33) |
| ADI-29424 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | DSFITFGGGTKVEIK |
| | (SEQ ID NO: 34) | (SEQ ID NO: 35) |
| ADI-29425 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | QSYPTFGGGTKVEIK |
| | (SEQ ID NO: 36) | (SEQ ID NO: 37) |
| ADI-29426 | QVQLQQWGAGLLKPSETLSLTCAV | DIQMTQSPSTLSASVGDRVTITC |
| | YGGSFSGYYWSWIRQPPGKGLEWI | RASQSIGSWLAWYQQKPGKAP |
| | GEIDHSGSTNYNPSLKSRVTISVDTS | KLLIYKASSLESGVPSRFSGSGS |
| | KNQFSLKLSSVTAADTAVYYCARA | GTEFTLTISSLQPDDFATYYCQQ |
| | RGPWSFDPWGQGTLVTVSS | YHSFPTFGGGTKVEIK |
| | (SEQ ID NO: 38) | (SEQ ID NO: 39) |
| ADI-29429 | QVQLQQWGAGLLKPSETLSLTCAV | DIQMTQSPSTLSASVGDRVTITC |
| | YGGSFSGYYWSWIRQPPGKGLEWI | RASQSIGSWLAWYQQKPGKAP |
| | GEIDHSGSTNYNPSLKSRVTISVDTS | KLLIYKASSLESGVPSRFSGSGS |
| | KNQFSLKLSSVTAADTAVYYCARA | GTEFTLTISSLQPDDFATYYCQQ |
| | RGPWSFDPWGQGTLVTVSS | YELYSYTFGGGTKVEIK |
| | (SEQ ID NO: 40) | (SEQ ID NO: 41) |
| ADI-29447 | QVQLQQWGAGLLKPSETLSLTCAV | DIQMTQSPSTLSASVGDRVTITC |
| (F47) | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | DTFITFGGGTKVEIK |
| | (SEQ ID NO: 42) | (SEQ ID NO: 43) |
| ADI-27727 | QVQLVQSGAEVKKPGSSVKVSCKA | DIVMTQSPDSLAVSLGERATINC |
| | SGGTFSSYAISWVRQAPGQGLEWM | KSSQSVLYSSNNKNYLAWYQQ |
| | GGIIPIFGTANYAQKFQGRVTITADE | KPGQPPKLLIYWASTRESGVPD |
| | STSTAYMELSSLRSEDTAVYYCAR | RFSGSGSGTDFTLTISSLQAEDV |
| | GDSSIRHAYYYYGMDVWGQGTTV | AVYYCQQYYSTPITFGGGTKVE |
| | TVSS | IK |
| | (SEQ ID NO: 44) | (SEQ ID NO: 48) |
| | CDR1 (SEQ ID NO: 45) - | CDR1 (SEQ ID NO: 49) - |
| | GTFSSYAIS (non-Kabat) or SYAIS | KSSQSVLYSSNNKNYLA |
| | (SEQ ID NO: 158) | CDR2 (SEQ ID NO: 50) - |
| | CDR2 (SEQ ID NO: 46) - | WASTRES |
| | GIIPIFGTANYAQKFQG | CDR3 (SEQ ID NO: 51) - |
| | CDR3 (SEQ ID NO: 47) - | QQYYSTPIT |
| | ARGDSSIRHAYYYYGMDV (non- | |
| | Kabat) or GDSSIRHAYYYYGMDV | |
| | (SEQ ID NO: 159) | |
| ADI-29443 | QLQLQESGPGLVKPSETLSLTCTVS | EIVLTQSPATLSLSPGERATLSCR |
| (F43) | GGSISSSSYYWGWIRQPPGKGLEWI | ASQSVSRYLAWYQQKPGQAPR |
| | GSIYYSGSTYYNPSLKSRVTISVDTS | LLIYDASNRATGIPARFSGSGSG |
| | KNQFSLKLSSVTAADTAVYYCARG | TDFTLTISSLEPEDFAVYYCQQF |
| | SDRFHPYFDYWGQGTLVTVSS | DTWPPTFGGGTKVEIK |
| | (SEQ ID NO: 52) | (SEQ ID NO: 56) |
| | CDR1 (SEQ ID NO: 53) - | CDR1 (SEQ ID NO: 57) - |
| | GSISSSSYYWG (non-Kabat) or | RASQSVSRYLA |
| | SSSYYWG (SEQ ID NO: 160) CDR2 | CDR2 (SEQ ID NO: 58) - |
| | (SEQ ID NO: 54) - | DASNRAT |
| | SIYYSGSTYYNPSLKS | CDR3 (SEQ ID NO: 59) - |
| | CDR3 (SEQ ID NO: 55) - | QQFDTWPPT |
| | ARGSDRFHPYPDY (non-Kabat) or | |
| | GSDRFHPYFDY (SEQ ID NO: 161) | |
| ADI-29404 | QVQLQQWGAGLLKPSETLSLTCAV | DIQMTQSPSTLSASVGDRVTITC |
| (F04) | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCEQY |
| | RGPWSFDPWGQGTLVTVSS | DSYPTFGGGTKVEIK |
| | (SEQ ID NO: 60) | (SEQ ID NO: 61) |
| ADI-28200 | QVQLVQSGAEVKKPGSSVKVSCKA | DIVMTQSPDSLAVSLGERATINC |
| | SGGTFSSYAISWVRQAPGQGLEWM | ESSQSLLNSGNQKNYLTWYQQ |
| | GGIIPIFGTANYAQKFQGRVTITADE | KPGQPPKPLIYWASTRESGVPD |
| | STSTAYMELSSLRSEDTAVYYCAR | RFSGSGSGTDFTLTISSLQAEDV |
| | RGRKASGSFYYYYGMDVWGQGTT | AVYYCQNDYSYPYTFGQGTKL |
| | VTVSS | EIK |
| | (SEQ ID NO: 62) | (SEQ ID NO: 66) |
| | CDR1 (SEQ ID NO: 63) - | CDR1 (SEQ ID NO: 67) - |
| | GTFSSYAIS (non-Kabat) or SYAIS | ESSQSLLNSGNQKNYLT |
| | (SEQ ID NO: 158) CDR2 (SEQ ID | CDR2 (SEQ ID NO: 68) - |
| | NO: 64) - GIIPIFGTANYAQKFQG | WASTRES |
| | CDR3 (SEQ ID NO: 65) - | CDR3 (SEQ ID NO: 69) - |
| | ARRGRKASGSFYYYYGMDV | QNDYSYPYT |
| ADI-29379 | QVQLVQSGAEVKKPGASVKVSCK | EIVMTQSPATLSVSPGERATLSC |
| (E79) | ASGYTFTSYYMHWVRQAPGQGLE | RASQSVSSNLAWYQQKPGQAP |
| | WMGIINPSGGSTSYAQKFQGRVTM | RLLIYGASTRATGIPARFSGSGS |
| | TRDTSTSTVYMELSSLRSEDTAVYY | GTEFTLTISSLQSEDFAVYYCQQ |
| | CARGAPNYGDTTHDYYYMDVWG | YDDWPFTFGGGTKVEIK |
| | KGTTVTVSS | (SEQ ID NO: 74) |
| | (SEQ ID NO: 70) | CDR1 (SEQ ID NO: 75) - |
| | CDR1 (SEQ ID NO: 71) - | RASQSVSSNLA |
| | YTFTSYYMH (non-Kabat) or SYYMH | CDR2 (SEQ ID NO: 76) - |
| | (SEQ ID NO: 162) | GASTRAT |
| | CDR2 (SEQ ID NO: 72) - | CDR3 (SEQ ID NO: 77) - |
| | IINPSGGSTSYAQKFQG | QQYDDWPFT |
| | CDR3 (SEQ ID NO: 73) - | |
| | ARGAPNYGDTTHDYYYMDV (non- | |
| | Kabat) or | |
| | GAPNYGDTTHDYYYMDV (SEQ ID | |
| | NO: 163) | |
| ADI-29463 | QVQLVQSGAEVKKPGASVKVSCK | EIVLTQSPGTLSLSPGERATLSCR |
| (F63) | ASGYTFTGYYMHWVRQAPGQGLE | ASQSVSSNLAWYQQKPGQAPR |
| | WMGWINPNSGGTNYAQKFQGRVT | LLIYGASTRATGIPARFSGSGSG |
| | MTRDTSISTAYMELSRLRSDDTAV | TEFTLTISSLQSEDFAVYYCQQD |
| | YYCARDTGEYYDTDDHGMDVWG | DYWPPTFGGGTKVEIK |
| | QGTTVTVSS | (SEQ ID NO: 82) |
| | (SEQ ID NO: 78) | CDR1 (SEQ ID NO: 75) - |
| | CDR1 (SEQ ID NO: 79) - | RASQSVSSNLA |
| | YTFTGYYMH (non-Kabat) or | CDR2 (SEQ ID NO: 76) - |
| | GYYMH (SEQ ID NO: 164) | GASTRAT |
| | CDR2 (SEQ ID NO: 80) - | CDR3 (SEQ ID NO: 85) - |
| | WINPNSGGTNYAQKFQG | QQDDYWPPT |
| | CDR3 (SEQ ID NO: 81) - | |
| | ARDTGEYYDTDDHGMDV (non- | |
| | Kabat) or DTGEYYDTDDHGMDV | |
| | (SEQ ID NO: 165) | |
| ADI-27744 | EVQLLESGGGLVQPGGSLRLSCAAS | DIQMTQSPSSVSASVGDRVTITC |
| (A44) | GFTFSSYAMSWVRQAPGKGLEWV | RASQGIDSWLAWYQQKPGKAP |
| | SAISGSGGSTYYADSVKGRFTISRD | KLLIYAASSLQSGVPSRFSGSGS |
| | NSKNTLYLQMNSLRAEDTAVYYC | GTDFTLTISSLQPEDFATYYCQQ |
| | AKDGGYYDSGAGDYWGQGTLVTV | GVSYPRTFGGGTKVEIK |
| | SS | (SEQ ID NO: 90) |
| | (SEQ ID NO: 86) | CDR1 (SEQ ID NO: 91) - |
| | CDR1 (SEQ ID NO: 87) - | RASQGIDSWLA |
| | FTFSSYAMS (non-Kabat) or SYAMS | CDR2 (SEQ ID NO: 92) - |
| | (SEQ ID NO: 166) | AASSLQS |
| | CDR2 (SEQ ID NO: 88) - | CDR3 (SEQ ID NO: 93) - |
| | AISGSGGSTYYADSVKG | QQGVSYPRT |
| | CDR3 (SEQ ID NO: 89) - | |
| | AKDGGYYDSGAGDY (non-Kabat) or | |
| | DGGYYDSGAGDY (SEQ ID NO: 167) | |
| ADI-27749 | EVQLVESGGGLVKPGGSLRLSCAA | DIQMTQSPSSVSASVGDRVTITC |
| (A49) | SGFTFSSYSMNWVRQAPGKGLEW | RASQGISSWLAWYQQKPGKAP |
| | VSSISSSSSYIYYADSVKGRFTISRD | KLLIYAASSLQSGVPSRFSGSGS |
| | NAKNSLYLQMNSLRAEDTAVYYC | GTDFTLTISSLQPEDFATYYCQQ |
| | ARGAPMGAAAGWFDPWGQGTLVT | GVSFPRTFGGGTKVEIK |
| | VSS | (SEQ ID NO: 98) |
| | (SEQ ID NO: 94) | CDR1 (SEQ ID NO: 99) - |
| | CDR1 (SEQ ID NO: 95) - | RASQGISSWLA |
| | FTFSSYSMN (non-Kabat) or SYSMN | CDR2 (SEQ ID NO: 100) - |
| | (SEQ ID NO: 168) | AASSLQS |
| | CDR2 (SEQ ID NO: 96) - | CDR3 (SEQ ID NO: 101) - |
| | SISSSSSYIYYADSVKG | QQGVSFPRT |
| | CDR3 (SEQ ID NO: 97) - | |
| | ARGAPMGAAAGWFDP (non-Kabat) | |
| | or GAPMGAAAGWFDP (SEQ ID | |
| | NO: 169) | |
| ADI-29378 | QVQLVQSGAEVKKPGASVKVSCK | EIVLTQSPATLSLSPGERATLSCR |
| (E78) | ASGYTFTSYYMHWVRQAPGQGLE | ASQSVSSYLAWYQQKPGQAPR |
| | WMGIINPSGGSTSYAQKFQGRVTM | LLIYDASNRATGIPARFSGSGSG |
| | TRDTSTSTVYMELSSLRSEDTAVYY | TDFTLTISSLEPEDFAVYYCQQS |
| | CAREGAGFAYGMDYYYMDVWGK | DNWPFTFGGGTKVEIK |
| | GTTVTVSS | (SEQ ID NO: 106) |
| | (SEQ ID NO: 102) | CDR1 (SEQ ID NO: 107) - |
| | CDR1 (SEQ ID NO: 71) - | RASQSVSSYLA |
| | YTFTSYYMH (non-Kabat) or | CDR2 (SEQ ID NO: 108) - |
| | SYYMH (SEQ ID NO: 162) | DASNRAT |
| | CDR2 (SEQ ID NO: 72) - | CDR3 (SEQ ID NO: 109) - |
| | IINPSGGSTSYAQKFQG | QQSDNWPFT |
| | CDR3 (SEQ ID NO: 105) - | |
| | AREGAGFAYGMDYYYMDV (non- | |
| | Kabat) or EGAGFAYGMDYYYMDV | |
| | (SEQ ID NO: 170) | |
| A49MI | EVQLVESGGGLVKPGGSLRLSCAA | DIQMTQSPSSVSASVGDRVTITC |
| | SGFTFSSYSMNWVRQAPGKGLEW | RASQGISSWLAWYQQKPGKAP |
| | VSSISSSSSYIYYADSVKGRFTISRD | KLLIYAASSLQSGVPSRFSGSGS |
| | NAKNSLYLQMNSLRAEDTAVYYC | GTDFTLTISSLQPEDFATYYCQQ |
| | ARGAPIGAAAGWFDPWGQGTLVT | GVSFPRTFGGGTKVEIK |
| | VSS | (SEQ ID NO: 98) |
| | (SEQ ID NO: 144) | CDR1 (SEQ ID NO: 99) - |
| | CDR1 (SEQ ID NO: 95) - | RASQGISSWLA |
| | FTFSSYSMN (non-Kabat) or SYSMN | CDR2 (SEQ ID NO: 100) - |
| | (SEQ ID NO: 168) | AASSLQS |
| | CDR2 (SEQ ID NO: 96) - | CDR3 (SEQ ID NO: 101) - |
| | SISSSSSYIYYADSVKG | QQGVSFPRT |
| | CDR3: (non-Kabat) | |
| | ARGAPIGAAAGWFDP (SEQ ID | |
| | NO: 172) or GAPIGAAAGWFDP (SEQ | |
| | ID NO: 173) | |
| A49MQ | EVQLVESGGGLVKPGGSLRLSCAA | DIQMTQSPSSVSASVGDRVTITC |
| | SGFTFSSYSMNWVRQAPGKGLEW | RASQGISSWLAWYQQKPGKAP |
| | VSSISSSSSYIYYADSVKGRFTISRD | KLLIYAASSLQSGVPSRFSGSGS |
| | NAKNSLYLQMNSLRAEDTAVYYC | GTDFTLTISSLQPEDFATYYCQQ |
| | ARGAPQGAAAGWFDPWGQGTLVT | GVSFPRTFGGGTKVEIK |
| | VSS | (SEQ ID NO: 98) |
| | (SEQ ID NO: 174) | CDR1 (SEQ ID NO: 99) - |
| | CDR1 (SEQ ID NO: 95) - | RASQGISSWLA |
| | FTFSSYSMN (non-Kabat) or SYSMN | CDR2 (SEQ ID NO: 100) - |
| | (SEQ ID NO: 168) | AASSLQS |
| | CDR2 (SEQ ID NO: 96) - | CDR3 (SEQ ID NO: 101) - |
| | SISSSSSYIYYADSVKG | QQGVSFPRT |
| | CDR3 (non-Kabat) (SEQ ID NO: 175) - | |
| | ARGAPQGAAAGWFDP or CDR3 | |
| | (SEQ ID NO: 176) - | |
| | GAPQGAAAGWFDP | |
| A49ML | EVQLVESGGGLVKPGGSLRLSCAA | DIQMTQSPSSVSASVGDRVTITC |
| | SGFTFSSYSMNWVRQAPGKGLEW | RASQGISSWLAWYQQKPGKAP |
| | VSSISSSSSYIYYADSVKGRFTISRD | KLLIYAASSLQSGVPSRFSGSGS |
| | NAKNSLYLQMNSLRAEDTAVYYC | GTDFTLTISSLQPEDFATYYCQQ |
| | ARGAPLGAAAGWPDPWGQGTLVT | GVSFPRTFGGGTKVEIK |
| | VSS | (SEQ ID NO: 98) |
| | (SEQ ID NO: 177) | CDR1 (SEQ ID NO: 99) - |
| | CDR1 (SEQ ID NO: 95) - | RASQGISSWLA |
| | FTFSSYSMN | CDR2 (SEQ ID NO: 100) - |
| | (SEQ ID NO: 168) | (non-Kabat) or SYSMN AASSLQS |
| | CDR2 (SEQ ID NO: 96) - | CDR3 (SEQ ID NO: 101) - |
| | SISSSSSYIYYADSVKG | QQGVSFPRT |
| | CDR3 (non-Kabat) (SEQ ID NO: 178) - | |
| | ARGAPLGAAAGWPDP or CDR3 | |
| | (SEQ ID NO: 179) - | |
| | GAPLGAAAGWFDP | |
| A49MF | EVQLVESGGGLVKPGGSLRLSCAA | DIQMTQSPSSVSASVGDRVTITC |
| | SGFTFSSYSMNWVRQAPGKGLEW | RASQGISSWLAWYQQKPGKAP |
| | VSSISSSSSYIYYADSVKGRFTISRD | KLLIYAASSLQSGVPSRFSGSGS |
| | NAKNSLYLQMNSLRAEDTAVYYC | GTDFTLTISSLQPEDFATYYCQQ |
| | ARGAPFGAAAGWFDPWGQGTLVT | GVSFPRTFGGGTKVEIK |
| | VSS | (SEQ ID NO: 98) |
| | (SEQ ID NO: 180) | CDR1 (SEQ ID NO: 99) - |
| | CDR1 (SEQ ID NO: 95) - | RASQGISSWLA |
| | FTFSSYSMN (non-Kabat) or SYSMN | CDR2 (SEQ ID NO: 100) - |
| | (SEQ ID NO: 168) | AASSLQS |
| | CDR2 (SEQ ID NO: 96) - | CDR3 (SEQ ID NO: 101) - |
| | SISSSSSYIYYADSVKG | QQGVSFPRT |
| | CDR3 (non-Kabat) (SEQ ID NO: 181) - | |
| | ARGAPFGAAAGWFDP or CDR3 | |
| | (SEQ ID NO: 182) - | |
| | GAPFGAAAGWFDP | |
| A49MV | EVQLVESGGGLVKPGGSLRLSCAA | DIQMTQSPSSVSASVGDRVTITC |
| | SGFTFSSYSMNWVRQAPGKGLEW | RASQGISSWLAWYQQKPGKAP |
| | VSSISSSSSYIYYADSVKGRFTISRD | KLLIYAASSLQSGVPSRFSGSGS |
| | NAKNSLYLQMNSLRAEDTAVYYC | GTDFTLTISSLQPEDFATYYCQQ |
| | ARGAPVGAAAGWFDPWGQGTLVT | GVSFPRTFGGGTKVEIK |
| | VSS | (SEQ ID NO: 98) |
| | (SEQ ID NO: 183) | CDR1 (SEQ ID NO: 99) - |
| | CDR1 (SEQ ID NO: 95) - | RASQGISSWLA |
| | FTFSSYSMN (non-Kabat) or SYSMN | CDR2 (SEQ ID NO: 100) - |
| | (SEQ ID NO: 168) | AASSLQS |
| | CDR2 (SEQ ID NO: 96) - | CDR3 (SEQ ID NO: 101) - |
| | SISSSSSYIYYADSVKG | QQGVSFPRT |
| | CDR3 (non-Kabat) (SEQ ID NO: 184) - | |
| | ARGAPVGAAAGWFDP or CDR3 | |
| | (SEQ ID NO: 185) - | |
| | GAPVGAAAGWFDP | |
| A49- | EVQLVESGGGLVKPGGSLRLSCAA | DIQMTQSPSSVSASVGDRVTITC |
| consensus | SGFTFSSYSMNWVRQAPGKGLEW | RASQGISSWLAWYQQKPGKAP |
| | VSSISSSSSYIYYADSVKGRFTISRD | KLLIYAASSLQSGVPSRFSGSGS |
| | NAKNSLYLQMNSLRAEDTAVYYC | GTDFTLTISSLQPEDFATYYCQQ |
| | ARGAPXGAAAGWFDPWGQGTLVT | GVSFPRTFGGGTKVEIK |
| | VSS, wherein X is M, L, I, V, Q, or F | (SEQ ID NO: 98) |
| | (SEQ ID NO: 186) | CDR1 (SEQ ID NO: 99) - |
| | CDR1 (SEQ ID NO: 95) - | RASQGISSWLA |
| | FTFSSYSMN (non-Kabat) or SYSMN | CDR2 (SEQ ID NO: 100) - |
| | (SEQ ID NO: 168) | AAS SLQS |
| | CDR2 (SEQ ID NO: 96) - | CDR3 (SEQ ID NO: 101) - |
| | SISSSSSYIYYADSVKG | QQGVSFPRT |
| | CDR3 (non-Kabat) (SEQ ID NO: 187) - | |
| | ARGAPXGAAAGWFDP or CDR3 | |
| | (SEQ ID NO: 188) - | |
| | GAPXGAAAGWFDP, wherein X is M, | |
| | L, I, V, Q, or F | |
| NKG2D | QVQLVESGGGLVKPGGSLRLSCAA | QSALTQPASVSGSPGQSITISCSG |
| binder in | SGFTFSSYGMHWVRQAPGKGLEW | SSSNIGNNAVNWYQQLPGKAPK |
| U.S. Pat. No. | VAFIRYDGSNKYYADSVKGRFTISR | LLIYYDDLLPSGVSDRFSGSKSG |
| 9,273,136 | DNSKNTLYLQMNSLRAEDTAVYY | TSAFLAISGLQSEDEADYYCAA |
| | CAKDRGLGDGTYFDYWGQGTTVT | WDDSLNGPVFGGGTKLTVL |
| | VS S(SEQ ID NO: 110) | (SEQ ID NO: 111) |
| NKG2D | QVHLQESGPGLVKPSETLSLTCTVS | EIVLTQSPGTLSLSPGERATLSCR |
| binder in | DDSISSYYWSWIRQPPGKGLEWIGH | ASQSVSSSYLAWYQQKPGQAPR |
| U.S. Pat. No. | ISYSGSANYNPSLKSRVTISVDTSKN | LLIYGASSRATGIPDRFSGSGSG |
| 7,879,985 | QFSLKLSSVTAADTAVYYCANWD | TDFTLTISRLEPEDFAVYYCQQY |
| | DAFNIWGQGTMVTVSS (SEQ ID | GSSPWTFGQGTKVEIK (SEQ ID |
| | NO: 112) | NO: 113) |

In some embodiments, the Fab comprises a heavy chain variable domain related to SEQ ID NO:94 and a light chain variable domain related to SEQ ID NO:98. For example, the heavy chain variable domain of the Fab can be at least 90% (e.g., 90%, 91%, 92%, 93%, 94%, 95%, 96%, 97%, 98%, 99%, or 100%) identical to SEQ ID NO:94, and/or incorporate amino acid sequences identical to the CDR1 (SEQ ID NO:95 or 168), CDR2 (SEQ ID NO:96), and CDR3 (SEQ ID NO:97 or 169) sequences of SEQ ID NO:94. Similarly, the light chain variable domain of the Fab can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO:101) sequences of SEQ ID NO:98.

In some embodiments, the Fab comprises a heavy chain variable domain related to SEQ ID NO:144 and a light chain variable domain related to SEQ ID NO:98. For example, the heavy chain variable domain of the Fab can be at least 90% (e.g., 90%, 91%, 92%, 93%, 94%, 95%, 96%, 97%, 98%, 99%, or 100%) identical to SEQ ID NO:144, and/or incorporate amino acid sequences identical to the CDR1 (SEQ ID NO:95 or 168), CDR2 (SEQ ID NO:96), and CDR3 (SEQ ID NO:172 or 173) sequences of SEQ ID NO:144. Similarly, the light chain variable domain of the Fab can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO:101) sequences of SEQ ID NO:98.

In some embodiments, the Fab comprises a heavy chain variable domain related to SEQ ID NO:174 and a light chain variable domain related to SEQ ID NO:98. For example, the heavy chain variable domain of the Fab can be at least 90% (e.g., 90%, 91%, 92%, 93%, 94%, 95%, 96%, 97%, 98%, 99%, or 100%) identical to SEQ ID NO:174, and/or incorporate amino acid sequences identical to the CDR1 (SEQ ID NO:95 or 168), CDR2 (SEQ ID NO:96), and CDR3 (SEQ ID NO:175 or 176) sequences of SEQ ID NO:174. Similarly, the light chain variable domain of the Fab can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO:101) sequences of SEQ ID NO:98.

In some embodiments, the Fab comprises a heavy chain variable domain related to SEQ ID NO:177 and a light chain variable domain related to SEQ ID NO:98. For example, the heavy chain variable domain of the Fab can be at least 90% (e.g., 90%, 91%, 92%, 93%, 94%, 95%, 96%, 97%, 98%, 99%, or 100%) identical to SEQ ID NO:177, and/or incorporate amino acid sequences identical to the CDR1 (SEQ ID NO:95 or 168), CDR2 (SEQ ID NO:96), and CDR3 (SEQ ID NO:178 or 179) sequences of SEQ ID NO:177. Similarly, the light chain variable domain of the Fab can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO:101) sequences of SEQ ID NO:98.

In some embodiments, the Fab comprises a heavy chain variable domain related to SEQ ID NO:180 and a light chain variable domain related to SEQ ID NO:98. For example, the heavy chain variable domain of the Fab can be at least 90% (e.g., 90%, 91%, 92%, 93%, 94%, 95%, 96%, 97%, 98%, 99%, or 100%) identical to SEQ ID NO:180, and/or incorporate amino acid sequences identical to the CDR1 (SEQ ID NO:95 or 168), CDR2 (SEQ ID NO:96), and CDR3 (SEQ ID NO:181 or 182) sequences of SEQ ID NO:180. Similarly, the light chain variable domain of the Fab can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO:101) sequences of SEQ ID NO:98.

In some embodiments, the Fab comprises a heavy chain variable domain related to SEQ ID NO:183 and a light chain variable domain related to SEQ ID NO:98. For example, the heavy chain variable domain of the Fab can be at least 90% (e.g., 90%, 91%, 92%, 93%, 94%, 95%, 96%, 97%, 98%, 99%, or 100%) identical to SEQ ID NO:183, and/or incorporate amino acid sequences identical to the CDR1 (SEQ ID NO:95 or 168), CDR2 (SEQ ID NO:96), and CDR3 (SEQ ID NO:184 or 185) sequences of SEQ ID NO:183. Similarly, the light chain variable domain of the Fab can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO:101) sequences of SEQ ID NO:98.

In some embodiments, the Fab comprises a heavy chain variable domain related to SEQ ID NO:186 and a light chain variable domain related to SEQ ID NO:98. For example, the heavy chain variable domain of the Fab can be at least 90% (e.g., 90%, 91%, 92%, 93%, 94%, 95%, 96%, 97%, 98%, 99%, or 100%) identical to SEQ ID NO:186, and/or incorporate amino acid sequences identical to the CDR1 (SEQ ID NO:95 or 168), CDR2 (SEQ ID NO:96), and CDR3 (SEQ ID NO:187 or 188) sequences of SEQ ID NO:186. Similarly, the light chain variable domain of the Fab can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO:101) sequences of SEQ ID NO:98.

In some embodiments, the Fab comprises a heavy chain variable domain related to SEQ ID NO:86 and a light chain variable domain related to SEQ ID NO:90. For example, the heavy chain variable domain of the Fab can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:87 or 166), CDR2 (SEQ ID NO:88), and CDR3 (SEQ ID NO:89 or 167) sequences of SEQ ID NO:86. Similarly, the light chain variable domain of the Fab can be at least 90% (e.g., 90%, 91%, 92%, 93%, 94%, 95%, 96%, 97%, 98%, 99%, or 100%) identical to SEQ ID NO:90, and/or incorporate amino acid sequences identical to the CDR1 (SEQ ID NO:91), CDR2 (SEQ ID NO:92), and CDR3 (SEQ ID NO:93) sequences of SEQ ID NO:90.

In some embodiments, the Fab comprises a heavy chain variable domain related to SEQ ID NO:102 and a light chain variable domain related to SEQ ID NO:106. For example, the heavy chain variable domain of the Fab can be at least 90% (e.g., 90%, 91%, 92%, 93%, 94%, 95%, 96%, 97%, 98%, 99%, or 100%) identical to SEQ ID NO:102, and/or incorporate amino acid sequences identical to the CDR1 (SEQ ID NO:71 or 162), CDR2 (SEQ ID NO:72), and CDR3 (SEQ ID NO:105 or 170) sequences of SEQ ID NO:102. Similarly, the light chain variable domain of the Fab can be at least 90% (e.g., 90%, 91%, 92%, 93%, 94%, 95%, 96%, 97%, 98%, 99%, or 100%) identical to SEQ ID NO:106, and/or incorporate amino acid sequences identical to the CDR1 (SEQ ID NO:107), CDR2 (SEQ ID NO:108), and CDR3 (SEQ ID NO:109) sequences of SEQ ID NO:106.

In some embodiments, the Fab comprises a heavy chain variable domain related to SEQ ID NO:70 and a light chain variable domain related to SEQ ID NO:74. For example, the heavy chain variable domain of the Fab can be at least 90% (e.g., 90%, 91%, 92%, 93%, 94%, 95%, 96%, 97%, 98%, 99%, or 100%) identical to SEQ ID NO:70, and/or incorporate amino acid sequences identical to the CDR1 (SEQ ID NO:71 or 162), CDR2 (SEQ ID NO:72), and CDR3 (SEQ ID NO:73 or 163) sequences of SEQ ID NO:70. Similarly, the light chain variable domain of the Fab can be at least 90% (e.g., 90%, 91%, 92%, 93%, 94%, 95%, 96%, 97%, 98%, 99%, or 100%) identical to SEQ ID NO:74, and/or incorporate amino acid sequences identical to the CDR1 (SEQ ID NO:75), CDR2 (SEQ ID NO:76), and CDR3 (SEQ ID NO:77) sequences of SEQ ID NO:74.

In some embodiments, the Fab comprises a heavy chain variable domain related to SEQ ID NO:70 and a light chain variable domain related to SEQ ID NO:74. For example, the heavy chain variable domain of the Fab can be at least 90% (e.g., 90%, 91%, 92%, 93%, 94%, 95%, 96%, 97%, 98%, 99%, or 100%) identical to SEQ ID NO:70, and/or incorporate amino acid sequences identical to the CDR1 (SEQ ID NO:71 or 162), CDR2 (SEQ ID NO:72), and CDR3 (SEQ ID NO:73 or 163) sequences of SEQ ID NO:70. Similarly, the light chain variable domain of the Fab can be at least 90% (e.g., 90%, 91%, 92%, 93%, 94%, 95%, 96%, 97%, 98%, 99%, or 100%) identical to SEQ ID NO:74, and/or incorporate amino acid sequences identical to the CDR1 (SEQ ID NO:75), CDR2 (SEQ ID NO:76), and CDR3 (SEQ ID NO:77) sequences of SEQ ID NO:74.

In some embodiments, the Fab comprises a heavy chain variable domain related to SEQ ID NO:78 and a light chain variable domain related to SEQ ID NO:82. For example, the heavy chain variable domain of the Fab can be at least 90% (e.g., 90%, 91%, 92%, 93%, 94%, 95%, 96%, 97%, 98%, 99%, or 100%) identical to SEQ ID NO:78, and/or incorporate amino acid sequences identical to the CDR1 (SEQ ID NO:79 or 164), CDR2 (SEQ ID NO:80), and CDR3 (SEQ ID NO:81 or 165) sequences of SEQ ID NO:78. Similarly, the light chain variable domain of the Fab can be at least 90% (e.g., 90%, 91%, 92%, 93%, 94%, 95%, 96%, 97%, 98%, 99%, or 100%) identical to SEQ ID NO:82, and/or incorporate amino acid sequences identical to the CDR1 (SEQ ID NO:75), CDR2 (SEQ ID NO:76), and CDR3 (SEQ ID NO:77) sequences of SEQ ID NO:82.

The multi-specific binding proteins can bind to NKG2D-expressing cells, which include but are not limited to NK cells, γδ T cells and CD8⁺ αβ T cells. Upon NKG2D binding, the multi-specific binding proteins may block natural ligands, such as ULBP6 and MICA, from binding to NKG2D and activating NK cells.

In certain embodiments, the Fab or the multi-specific binding protein binds to NKG2D with an affinity of K_{D} of 2 nM to 120 nM, e.g., 2 nM to 110 nM, 2 nM to 100 nM, 2 nM to 90 nM, 2 nM to 80 nM, 2 nM to 70 nM, 2 nM to 60 nM, 2 nM to 50 nM, 2 nM to 40 nM, 2 nM to 30 nM, 2 nM to 20 nM, 2 nM to 10 nM, about 15 nM, about 14 nM, about 13 nM, about 12 nM, about 11 nM, about 10 nM, about 9 nM, about 8 nM, about 7 nM, about 6 nM, about 5 nM, about 4.5 nM, about 4 nM, about 3.5 nM, about 3 nM, about 2.5 nM, about 2 nM, about 1.5 nM, about 1 nM, between about 0.5 nM to about 1 nM, about 1 nM to about 2 nM, about 2 nM to 3 nM, about 3 nM to 4 nM, about 4 nM to about 5 nM, about 5 nM to about 6 nM, about 6 nM to about 7 nM, about 7 nM to about 8 nM, about 8 nM to about 9 nM, about 9 nM to about 10 nM, about 1 nM to about 10 nM, about 2 nM to about 10 nM, about 3 nM to about 10 nM, about 4 nM to about 10 nM, about 5 nM to about 10 nM, about 6 nM to about 10 nM, about 7 nM to about 10 nM, or about 8 nM to about 10 nM.

In certain embodiments, the Fab binds to NKG2D with a K_{D} of 2 nM to 120 nM, as measured by surface plasmon resonance. In certain embodiments, the multi-specific binding protein binds to NKG2D with a K_{D} of 2 nM to 120 nM, as measured by surface plasmon resonance. In certain embodiments, the Fab binds to NKG2D with a K_{D} of 10 nM to 62 nM, as measured by surface plasmon resonance. In certain embodiments, the multi-specific binding protein binds to NKG2D with a K_{D} of 10 nM to 62 nM, as measured by surface plasmon resonance.

In some embodiments, the Fab described above is linked to an antibody Fc sequence. In some embodiments, the heavy chain portion of the Fab is linked to the N-terminus of an antibody Fc sequence.

### HER2-Binding Site

The HER2-binding site of the multi-specific binding protein disclosed herein comprises a heavy chain variable domain and a light chain variable domain fused together to from an scFv. Table 2 lists peptide sequences of heavy chain variable domains and light chain variable domains that, in combination, can bind to HER2.

TABLE 2
Exemplary HER2-Binding Sites

| | Heavy chain variable domain amino | Light chain variable domain amino |
|---|---|---|
| Clones | acid sequence | acid sequence |
| Trastuzumab | EVQLVESGGGLVQPGGSLRLSCA | DIQMTQSPSSLSASVGDRVTITCR |
| | ASGFNIKDTYIHWVRQAPGKGLE | ASQDVNTAVAWYQQKPGKAPK |
| | WVARIYPTNGYTRYADSVKGRFT | LLIYSASFLYSGVPSRFSGSRSGT |
| | ISADTSKNTAYLQMNSLRAEDTA | DFTLTISSLQPEDFATYYCQQHY |
| | VYYCSRWGGDGFYAMDYWGQG | TTPPTFGQGTKVEIK |
| | TLVTVSS | (SEQ ID NO: 118) |
| | (SEQ ID NO: 114) | CDR1(SEQ ID NO: 119) - |
| | CDR1(SEQ ID NO: 115) - GFNIKDT | QDVNTAVA |
| | CDR2 (SEQ ID NO: 116) - YPTNGY | CDR2 (SEQ ID NO: 120) - |
| | CDR3 (SEQ ID NO: 117) - | SASFLYS |
| | WGGDGFYAMDY | CDR3 (SEQ ID NO: 121) - |
| | | QQHYTTPPT |
| Trastuzumab | EVQLVESGGGLVQPGGSLRLSCA | DIQMTQSPSSLSASVGDRVTITCR |
| (VH and VL | ASGFNIKDTYIHWVRQAPGK [symbol] LE | ASQDVNTAVAWYQQKPGKAPK |
| in scFv | WVARIYPTNGYTRYADSVKGRFT | LLIYSASFLYSGVPSRFSGSRSGT |
| construct) | ISADTSKNTAYLQMNSLRAEDTA | DFTLTISSLQPEDFATYYCQQHY |
| | VYYCSRWGGDGFYAMDYWGQG | TTPPTFG [symbol] GTKVEIK |
| | TLVTVSS (SEQ ID NO: 195) | (SEQ ID NO: 196) |
| | CDR1(SEQ ID NO: 115) - GFNIKDT | CDR1(SEQ ID NO: 119) - |
| | CDR2 (SEQ ID NO: 116) - YPTNGY | QDVNTAVA |
| | CDR3 (SEQ ID NO: 117) - | CDR2 (SEQ ID NO: 120) - |
| | WGGDGFYAMDY | SASFLYS |
| | | CDR3 (SEQ ID NO: 121) - |
| | | QQHYTTPPT |
| Trastuzumab- | DIQMTQSPSSLSASVGDRVTITCRASQDVNTAVAWYQQKPGKAPKLLI | |
| scFv | YSASFLYSGVPSRFSGSRSGTDFTLTISSLQPEDFATYYCQQHYTTPPTF | |
| | GCGTKVEIKGGGGSGGGGSGGGGSGGGGSEVQLVESGGGLVQPGGSL | |
| | RLSCAASGFNIKDTYIHWVRQAPGKCLEWVARIYPTNGYTRYADSVK | |
| | GRFTISADTSKNTAYLQMNSLRAEDTAVYYCSRWGGDGFYAMDYWG | |
| | QGTLVTVSS(SEQ ID NO: 139) | |
| Pertuzumab | EVQLVESGGGLVQPGGSLRLSCA | DIQMTQSPSSLSASVGDRVTITCK |
| | ASGFTFTDYTMDWVRQAPGKGL | ASQDVSIGVAWYQQKPGKAPKL |
| | EWVADVNPNSGGSIYNQRFKGRF | LIYSASYRYTGVPSRFSGSGSGTD |
| | TLSVDRSKNTLYLQMNSLRAEDT | FTLTISSLQPEDFATYYCQQYYIY |
| | AVYYCARNLGPSFYFDYWGQGT | PYTFGQGTKVEIKR |
| | LVTVSSA | (SEQ ID NO: 126) |
| | (SEQ ID NO: 122) | CDR1 (SEQ ID NO: 127) - |
| | CDR1 (SEQ ID NO: 123) - GFTFTDY | QDVSIGVA |
| | CDR2 (SEQ ID NO: 124) - NPNSGG | CDR2 (SEQ ID NO: 128) - |
| | CDR3 (SEQ ID NO: 125) - | SASYRYT |
| | NLGPSFYFDY | CDR3 (SEQ ID NO: 129) - |
| | | QQYYIYPYT |
| Pertuzumab | EVQLVESGGGLVQPGGSLRLSCA | DIQMTQSPSSLSASVGDRVTITCK |
| (VH and VL | ASGFTFTDYTMDWVRQAPGK [symbol] L | ASQDVSIGVAWYQQKPGKAPKL |
| in scFv | EWVADVNPNSGGSIYNQRFKGRF | LIYSASYRYTGVPSRFSGSGSGTD |
| construct) | TLSVDRSKNTLYLQMNSLRAEDT | FTLTISSLQPEDFATYYCQQYYIY |
| | AVYYCARNLGPSFYFDYWGQGT | PYTFG [symbol] GTKVEIKR |
| | LVTVSSA (SEQ ID NO: 197) | (SEQ ID NO: 198) |
| | CDR1 (SEQ ID NO: 123) - GFTFTDY | CDR1 (SEQ ID NO: 127) - |
| | CDR2 (SEQ ID NO: 124) - NPNSGG | QDVSIGVA |
| | CDR3 (SEQ ID NO: 125) - | CDR2 (SEQ ID NO: 128) - |
| | NLGPSFYFDY | SASYRYT |
| | | CDR3 (SEQ ID NO: 129) - |
| | | QQYYIYPYT |
| Pertuzumab | DIQMTQSPSSLSASVGDRVTITCKASQDVSIGVAWYQQKPGKAPKLLI | |
| scFv | YSASYRYTGVPSRFSGSGSGTDFTLTISSLQPEDFATYYCQQYYIYPYTF | |
| | GCGTKVEIKRGGGGSGGGGSGGGGSGGGGSEVQLVESGGGLVQPGGS | |
| | LRLSCAASGFTFTDYTMDWVRQAPGKCLEWVADVNPNSGGSIYNQRF | |
| | KGRFTLSVDRSKNTLYLQMNSLRAEDTAVYYCARNLGPSFYFDYWG | |
| | QGTLVTVSSA | |
| | (SEQ ID NO: 189) | |
| MGAH22 | QVQLQQSGPELVKPGASLKLSCT | DIVMTQSHKFMSTSVGDRVSITC |
| (U.S. Pat. No. | ASGFNIKDTYIHWVKQRPEQGLE | KASQDVNTAVAWYQQKPGHSP |
| 8,802,093) | WIGRIYPTNGYTRYDPKFQDKATI | KLLIYSASFRYTGVPDRFTGSRSG |
| | TADTSSNTAYLQVSRLTSEDTAV | TDFTFTISSVQAEDLAVYYCQQH |
| | YYCSRWGGDGFYAMDYWGQGA | YTTPPTFGGGTKVEIK |
| | SVTVSS | (SEQ ID NO: 134) |
| | (SEQ ID NO: 130) | CDR1 (SEQ ID NO: 135) - |
| | CDR1 (SEQ ID NO: 131) - GFNIKDT | QDVNTAVA |
| | CDR2 (SEQ ID NO: 132) - YPTNGY | CDR2 (SEQ ID NO: 136) - |
| | CDR3 (SEQ ID NO: 133) - | SASFRYT |
| | WGGDGFYAMDY | CDR3 (SEQ ID NO: 137) - |
| | | QQHYTTPPT |
| MGAH22 | QVQLQQSGPELVKPGASLKLSCT | DIVMTQSHKFMSTSVGDRVSITC |
| (VH and VL | ASGFNIKDTYIHWVKQRPEQ [symbol] LE | KASQDVNTAVAWYQQKPGHSP |
| in scFv | WIGRIYPTNGYTRYDPKFQDKATI | KLLIYSASFRYTGVPDRFTGSRSG |
| construct) | TADTSSNTAYLQVSRLTSEDTAV | TDFTFTISSVQAEDLAVYYCQQH |
| | YYCSRWGGDGFYAMDYWGQGA | YTTPPTFG [symbol] GTKVEIKR (SEQ ID |
| | SVTVSSA (SEQ ID NO: 199) | NO: 200) |
| | CDR1 (SEQ ID NO: 131) - GFNIKDT | CDR1 (SEQ ID NO: 135) - |
| | CDR2 (SEQ ID NO: 132) - YPTNGY | QDVNTAVA |
| | CDR3 (SEQ ID NO: 133) - | CDR2 (SEQ ID NO: 136) - |
| | WGGDGFYAMDY | SASFRYT |
| | | CDR3 (SEQ ID NO: 137) - |
| | | QQHYTTPPT |

| | |
|---|---|
| MGAH22 | DIVMTQSHKFMSTSVGDRVSITCKASQDVNTAVAWYQQKPGHSPKLL |
| scFv | IYSASFRYTGVPDRFTGSRSGTDFTFTISSVQAEDLAVYYCQQHYTTPP |
| | TFG [symbol] GTKVEIKRGGGGSGGGGSGGGGSGGGGSQVQLQQSGPELVKPG |
| | ASLKLSCTASGFNIKDTYIHWVKQRPEQ [symbol] LEWIGRIYPTNGYTRYDPKF |
| | QDKATITADTSSNTAYLQVSRLTSEDTAVYYCSRWGGDGFYAMDYW |
| | GQGASVTVSSA |
| | (SEQ ID NO: 171) |

Alternatively, novel antigen-binding sites that can bind to HER2 can be identified by screening for binding to the amino acid sequence defined by SEQ ID NO:138 or a mature extracellular fragment thereof.

(SEQ ID NO: 138)
MELAALCRWGLLLALLPPGAASTQVCTGTDMKLRLPASPETHLDMLRHLY
QGCQVVQGNLELTYLPTNASLSFLQDIQEVQGYVLIAHNQVRQVPLQRLR
IVRGTQLFEDNYALAVLDNGDPLNNTTPVTGASPGGLRELQLRSLTEILK
GGVLIQRNPQLCYQDTILWKDIFHKNNQLALTLIDTNRSRACHPCSPMCK
GSRCWGESSEDCQSLTRTVCAGGCARCKGPLPTDCCHEQCAAGCTGPKHS
DCLACLHFNHSGICELHCPALVTYNTDTFESMPNPEGRYTFGASCVTACP
YNYLSTDVGSCTLVCPLHNQEVTAEDGTQRCEKCSKPCARVCYGLGMEHL
REVRAVTSANIQEFAGCKKIFGSLAFLPESFDGDPASNTAPLQPEQLQVF
ETLEEITGYLYISAWPDSLPDLSVFQNLQVIRGRILHNGAYSLTLQGLGI
SWLGLRSLRELGSGLALIHHNTHLCFVHTVPWDQLFRNPHQALLHTANRP
EDECVGEGLACHQLCARGHCWGPGPTQCVNCSQFLRGQECVEECRVLQGL
PREYVNARHCLPCHPECQPQNGSVTCFGPEADQCVACAHYKDPPFCVARC
PSGVKPDLSYMPIWKFPDEEGACQPCPINCTHSCVDLDDKGCPAEQRASP
LTSIISAVVGILLVVVLGVVFGILIKRRQQKIRKYTMRRLLQETELVEPL
TPSGAMPNQAQMRILKETELRKVKVLGSGAFGTVYKGIWIPDGENVKIPV
AIKVLRENTSPKANKEILDEAYVMAGVGSPYVSRLLGICLTSTVQLVTQL
MPYGCLLDHVRENRGRLGSQDLLNWCMQIAKGMSYLEDVRLVHRDLAARN
VLVKSPNHVKITDFGLARLLDIDETEYHADGGKVPIKWMALESILRRRFT
HQSDVWSYGVTVWELMTFGAKPYDGIPAREIPDLLEKGERLPQPPICTID
VYMIMVKCWMIDSECRPRFRELVSEFSRMARDPQRFVVIQNEDLGPASPL
DSTFYRSLLEDDDMGDLVDAEEYLVPQQGFFCPDPAPGAGGMVHHRHRSS
STRSGGGDLTLGLEPSEEEAPRSPLAPSEGAGSDVFDGDLGMGAAKGLQS
LPTHDPSPLQRYSEDPTVPLPSETDGYVAPLTCSPQPEYVNQPDVRPQPP
SPREGPLPAARPAGATLERPKTLSPGKNGVVKDVFAFGGAVENPEYLTPQ
GGAAPQPHPPPAFSPAFDNLYYWDQDPPERGAPPSTFKGTPTAENPEYLG
LDVPV

In some embodiments, the scFv comprises a heavy chain variable domain related to SEQ ID NO:195 and a light chain variable domain related to SEQ ID NO:196. For example, the heavy chain variable domain of the scFv can be at least 90% (e.g., 90%, 91%, 92%, 93%, 94%, 95%, 96%, 97%, 98%, 99%, or 100%) identical to SEQ ID NO:195, and/or incorporate amino acid sequences identical to the CDR1 (SEQ ID NO:115), CDR2 (SEQ ID NO:116), and CDR3 (SEQ ID NO:117) sequences of SEQ ID NO:195. Similarly, the light chain variable domain of the scFv can be at least 90% (e.g., 90%, 91%, 92%, 93%, 94%, 95%, 96%, 97%, 98%, 99%, or 100%) identical to SEQ ID NO:196, and/or incorporate amino acid sequences identical to the CDR1 (SEQ ID NO:119), CDR2 (SEQ ID NO:120), and CDR3 (SEQ ID NO:121) sequences of SEQ ID NO:196. In some embodiments, the scFv comprises the amino acid sequence of SEQ ID NO:139.

In some embodiments, the scFv comprises a heavy chain variable domain related to SEQ ID NO:197 and a light chain variable domain related to SEQ ID NO:198. For example, the heavy chain variable domain of the scFv can be at least 90% (e.g., 90%, 91%, 92%, 93%, 94%, 95%, 96%, 97%, 98%, 99%, or 100%) identical to SEQ ID NO:197, and/or incorporate amino acid sequences identical to the CDR1 (SEQ ID NO:123), CDR2 (SEQ ID NO:124), and CDR3 (SEQ ID NO:125) sequences of SEQ ID NO:197. Similarly, the light chain variable domain of the scFv can be at least 90% (e.g., 90%, 91%, 92%, 93%, 94%, 95%, 96%, 97%, 98%, 99%, or 100%) identical to SEQ ID NO:198, and/or incorporate amino acid sequences identical to the CDR1 (SEQ ID NO:127), CDR2 (SEQ ID NO:128), and CDR3 (SEQ ID NO:129) sequences of SEQ ID NO:198. In some embodiments, the scFv comprises the amino acid sequence of SEQ ID NO:189.

In some embodiments, the scFv comprises a heavy chain variable domain related to SEQ ID NO:199 and a light chain variable domain related to SEQ ID NO:200. For example, the heavy chain variable domain of the scFv can be at least 90% (e.g., 90%, 91%, 92%, 93%, 94%, 95%, 96%, 97%, 98%, 99%, or 100%) identical to SEQ ID NO:199, and/or incorporate amino acid sequences identical to the CDR1 (SEQ ID NO:131), CDR2 (SEQ ID NO:132), and CDR3 (SEQ ID NO:133) sequences of SEQ ID NO:199. Similarly, the light chain variable domain of the scFv can be at least 90% (e.g., 90%, 91%, 92%, 93%, 94%, 95%, 96%, 97%, 98%, 99%, or 100%) identical to SEQ ID NO:200, and/or incorporate amino acid sequences identical to the CDR1 (SEQ ID NO:135), CDR2 (SEQ ID NO:136), and CDR3 (SEQ ID NO:137) sequences of SEQ ID NO:200. In some embodiments, the scFv comprises the amino acid sequence of SEQ ID NO:171.

The scFv described above includes a heavy chain variable domain and a light chain variable domain. In some embodiments, the heavy chain variable domain forms a disulfide bridge with the light chain variable domain to enhance stability of the scFv. For example, a disulfide bridge can be formed between the C44 residue of the heavy chain variable domain and the C100 residue of the light chain variable domain, the amino acid positions numbered under Kabat.

The VH and VL of the scFv can be positioned in various orientations. In certain embodiments, the VL is positioned N-terminal to the VH. In certain embodiments, the VL is positioned C-terminal to the VH.

The VH and VL of the scFv can be connected via a linker, e.g., a peptide linker. In certain embodiments, the peptide linker is a flexible linker. Regarding the amino acid composition of the linker, peptides are selected with properties that confer flexibility, do not interfere with the structure and function of the other domains of the proteins of the present invention, and resist cleavage from proteases. For example, glycine and serine residues generally provide protease resistance. In certain embodiments, the VL is positioned N-terminal to the VH and is connected to the VH via a linker.

The length of the linker (e.g., flexible linker) can be “short,” e.g., 0, 1, 2, 3, 4, 5, 6, 7, 8, 9, 10, 11 or 12 amino acid residues, or “long,” e.g., at least 13 amino acid residues. In certain embodiments, the linker is 10-50, 10-40, 10-30, 10-25, 10-20, 15-50, 15-40, 15-30, 15-25, 15-20, 20-50, 20-40, 20-30, or 20-25 amino acid residues in length.

In certain embodiments, the linker comprises or consists of a (GS)ₙ (SEQ ID NO:204), (GGS)ₙ (SEQ ID NO:205), (GGGS)ₙ(SEQ ID NO:151), (GGSG)ₙ (SEQ ID NO:153), (GGSGG)ₙ (SEQ ID NO:156), and (GGGGS)ₙ (SEQ ID NO:157) sequence, wherein n is 1, 2, 3, 4, 5, 6, 7, 8, 9, 10, 11, 12, 13, 14, 15, 16, 17, 18, 19, or 20. In certain embodiments, the linker comprises or consists of an amino acid sequence selected from SEQ ID NO:143, SEQ ID NO:201, SEQ ID NO:202, SEQ ID NO: 103, SEQ ID NO:104, SEQ ID NO:83, SEQ ID NO:84, SEQ ID NO:150, SEQ ID NO:152, and SEQ ID NO:154, as listed in Table 3. In certain embodiments, the linker is a (G₄S)₄ (SEQ ID NO:203) linker consisting of the sequence of SEQ ID NO:143.

TABLE 3
Exemplary Linkers

| SEQ ID | Amino Acid Sequence |
|---|---|
| SEQ ID | GSGSGSGSGSGSGSGSGSGS |
| NO: 201 | |
| SEQ ID | GGSGGSGGSGGSGGSGGSGGSGGSGGSGGS |
| NO: 202 | |
| SEQ ID | GGGSGGGSGGGSGGGSGGGSGGGSGGGSGGGSGGGS |
| NO: 103 | GGGS |
| SEQ ID | GGSGGGSGGGSGGGSGGGSGGGSGGGSGGGSGGGSG |
| NO: 104 | GGSG |
| SEQ ID | GGSGGGGSGGGGSGGGGSGGGGSGGGGSGGGGSGG |
| NO: 83 | GGSGGGGSGGGGSGG |
| SEQ ID | GGGGSGGGGSGGGGSGGGGSGGGGSGGGGSGGGGS |
| NO: 84 | GGGGSGGGGSGGGGS |
| SEQ ID | GGGGSGGGGSGGGGSGGGGS |
| NO: 143 | |
| SEQ ID | GGGGSGGGGSGGGGS |
| NO: 150 | |
| SEQ ID | GGGGSGGGGSGGGGSGGGGSGGGGSGGGGSGGGGS |
| NO: 152 | GGGGSGGGGSGGGGSGGGGSGGGGSGGGGSGGGGS |
| | GGGGSGGGGSGGGGSGGGGSGGGGSGGGGS |
| SEQ ID | GGSGGGGSGGGGSGGGGSGGGGSGGGGSGGGGSGG |
| NO: 154 | GGSGGGGSGGGGSGGGGSGGGGSGGGGSGGGGSGG |
| | GGSGGGGSGGGGSGGGGSGGGGSGGGGSGG |

In specific embodiments, the light chain variable domain is linked to the N-terminus of the heavy chain variable domain via a flexible linker, e.g., the (G₄S)₄ linker (SEQ ID NO:203).

In some embodiments, the scFv described above is linked to an antibody Fc sequence via a hinge sequence. In some embodiments, the hinge comprises the amino acids Ala-Ser. In some other embodiments, the hinge comprises the amino acids Ala-Ser and Thr-Lys-Gly. The hinge sequence can provide flexibility of binding to the target antigen and balance between flexibility and optimal geometry.

### Fc Domain

The antibody Fc domain of the multi-specific binding protein comprises a first antibody Fc sequence linked to the Fab and a second antibody Fc sequence linked to the scFv. The two antibody Fc sequences pair and form a dimer that binds CD16.

Within the antibody Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

The assembly of heterodimeric antibody heavy chains can be accomplished by expressing two different antibody heavy chain sequences in the same cell, which may lead to the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. Nos. 13/494,870, 16/028,850, 11/533,709, 12/875,015, 13/289,934, 14/773,418, 12/811,207, 13/866,756, 14/647,480, and 14/830,336. For example, mutations can be made in the CH3 domain based on human IgG1 through incorporating distinct pairs of amino acid substitutions within a first polypeptide and a second polypeptide that allow these two chains to selectively heterodimerize with each other. The positions of amino acid substitutions illustrated below are all numbered according to the EU index as in Kabat.

In one scenario, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V.

An antibody heavy chain variable domain of the invention can optionally be coupled to an amino acid sequence at least 90% identical to an antibody constant region, such as an IgG constant region including hinge, CH2 and CH3 domains with or without CH1 domain. In some embodiments, the amino acid sequence of the constant region is at least 90% identical to a human antibody constant region, such as a human IgG1 constant region, an IgG2 constant region, IgG3 constant region, or IgG4 constant region. In some other embodiments, the amino acid sequence of the constant region is at least 90% identical to an antibody constant region from another mammal, such as rabbit, dog, cat, mouse, or horse. One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, T411D, T411E, K439D, and K439E. All the amino acid positions in an Fc domain or hinge region disclosed herein are numbered according to EU numbering.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cκ of a human IgG1 constant region may be at amino acid E123, F116, S176, V163, S174, and/or T164.

Amino acid substitutions could be selected from the following sets of substitutions shown in Table 4.

TABLE 4
Exemplary Fc Substitutions that Promote Heterodimerization

| | First Polypeptide | Second Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K3705 |
| Set 9 | L368D/K3705 | S364K |
| Set 10 | L368E/K3705 | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 5.

TABLE 5
Exemplary Fc Substitutions that Promote Heterodimerization

| | First Polypeptide | Second Polypeptide |
|---|---|---|
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following sets of substitutions shown in Table 6.

TABLE 6
Exemplary Fc Substitutions that Promote Heterodimerization

| | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 7.

TABLE 7
Exemplary Fc Substitutions that Promote Heterodimerization

| First Polypeptide | Second Polypeptide |
|---|---|
| L351Y, D399R, D399K, | T366V, T3661, T366L, T366M, N390D, |
| S400K, S400R, Y407A, | N390E, K392L, K392M, K392V, K392F |
| Y407I, | Y407V K392D, K392E, K409F, K409W, |
| | T411D and T411E |

Alternatively, at least one amino acid substitution could be selected from the following sets of substitutions in Table 8, where the position(s) indicated in the First Polypeptide column is replaced by any known negatively-charged amino acid, and the position(s) indicated in the Second Polypeptide Column is replaced by any known positively-charged amino acid.

TABLE 8
Exemplary Fc Positions for Substitutions

| First Polypeptide | Second Polypeptide |
|---|---|
| K392, K370, K409, or K439 | D399, E356, or E357 |

Alternatively, at least one amino acid substitution could be selected from the following sets of substitutions in Table 9, where the position(s) indicated in the First Polypeptide column is replaced by any known positively-charged amino acid, and the position(s) indicated in the Second Polypeptide Column is replaced by any known negatively-charged amino acid.

TABLE 9
Exemplary Fc Positions for Substitutions

| First Polypeptide | Second Polypeptide |
|---|---|
| D399, E356, or E357 | K409, K439, K370, or K392 |

Alternatively, amino acid substitutions could be selected from the following sets in Table 10.

TABLE 10
Exemplary Fc Substitutions that Promote Heterodimerization

| First Polypeptide | Second Polypeptide |
|---|---|
| T350V, L351Y, F405A, | T350V, T366L, K392L, |
| and Y407V | and T394W |

When selecting Fc substitutions, a skilled person would appreciate that the first polypeptide and the second polypeptide in Tables 4-10 may correspond to the first antibody Fc sequence and the second antibody Fc sequence, respectively. Alternatively, the first polypeptide and the second polypeptide in Tables 4-10 may correspond to the second antibody Fc sequence and the first antibody Fc sequence, respectively.

In some embodiments, the amino acid sequence of one polypeptide chain of the antibody constant region differs from the amino acid sequence of an IgG1 constant region at position T366, and wherein the amino acid sequence of the other polypeptide chain of the antibody constant region differs from the amino acid sequence of an IgG1 constant region at one or more positions selected from the group consisting of T366, L368 and Y407.

In some embodiments, the amino acid sequence of one polypeptide chain of the antibody constant region differs from the amino acid sequence of an IgG1 constant region at one or more positions selected from the group consisting of T366, L368 and Y407, and wherein the amino acid sequence of the other polypeptide chain of the antibody constant region differs from the amino acid sequence of an IgG1 constant region at position T366.

In some embodiments, the amino acid sequence of one polypeptide chain of the antibody constant region differs from the amino acid sequence of an IgG1 constant region at one or more positions selected from the group consisting of E357, K360, Q362, S364, L368, K370, T394, D401, F405, and T411 and wherein the amino acid sequence of the other polypeptide chain of the antibody constant region differs from the amino acid sequence of an IgG1 constant region at one or more positions selected from the group consisting of Y349, E357, S364, L368, K370, T394, D401, F405 and T411.

In some embodiments, the amino acid sequence of one polypeptide chain of the antibody constant region differs from the amino acid sequence of an IgG1 constant region at one or more positions selected from the group consisting of Y349, E357, S364, L368, K370, T394, D401, F405 and T411 and wherein the amino acid sequence of the other polypeptide chain of the antibody constant region differs from the amino acid sequence of an IgG1 constant region at one or more positions selected from the group consisting of E357, K360, Q362, S364, L368, K370, T394, D401, F405, and T411.

In some embodiments, the amino acid sequence of one polypeptide chain of the antibody constant region differs from the amino acid sequence of an IgG1 constant region at one or more positions selected from the group consisting of L351, D399, S400 and Y407 and wherein the amino acid sequence of the other polypeptide chain of the antibody constant region differs from the amino acid sequence of an IgG1 constant region at one or more positions selected from the group consisting of T366, N390, K392, K409 and T411.

In some embodiments, the amino acid sequence of one polypeptide chain of the antibody constant region differs from the amino acid sequence of an IgG1 constant region at one or more positions selected from the group consisting of T366, N390, K392, K409 and T411 and wherein the amino acid sequence of the other polypeptide chain of the antibody constant region differs from the amino acid sequence of an IgG1 constant region at one or more positions selected from the group consisting of L351, D399, S400 and Y407.

In some embodiments, the amino acid sequence of one polypeptide chain of the antibody constant region differs from the amino acid sequence of an IgG1 constant region at one or more positions selected from the group consisting of Q347, Y349, K360, and K409, and wherein the amino acid sequence of the other polypeptide chain of the antibody constant region differs from the amino acid sequence of an IgG1 constant region at one or more positions selected from the group consisting of Q347, E357, D399 and F405.

In some embodiments, the amino acid sequence of one polypeptide chain of the antibody constant region differs from the amino acid sequence of an IgG1 constant region at one or more positions selected from the group consisting of Q347, E357, D399 and F405, and wherein the amino acid sequence of the other polypeptide chain of the antibody constant region differs from the amino acid sequence of an IgG1 constant region at one or more positions selected from the group consisting of Y349, K360, Q347 and K409.

In some embodiments, the amino acid sequence of one polypeptide chain of the antibody constant region differs from the amino acid sequence of an IgG1 constant region at one or more positions selected from the group consisting of K370, K392, K409 and K439, and wherein the amino acid sequence of the other polypeptide chain of the antibody constant region differs from the amino acid sequence of an IgG1 constant region at one or more positions selected from the group consisting of D356, E357 and D399.

In some embodiments, the amino acid sequence of one polypeptide chain of the antibody constant region differs from the amino acid sequence of an IgG1 constant region at one or more positions selected from the group consisting of D356, E357 and D399, and wherein the amino acid sequence of the other polypeptide chain of the antibody constant region differs from the amino acid sequence of an IgG1 constant region at one or more positions selected from the group consisting of K370, K392, K409 and K439.

In some embodiments, the amino acid sequence of one polypeptide chain of the antibody constant region differs from the amino acid sequence of an IgG1 constant region at one or more positions selected from the group consisting of L351, E356, T366 and D399, and wherein the amino acid sequence of the other polypeptide chain of the antibody constant region differs from the amino acid sequence of an IgG1 constant region at one or more positions selected from the group consisting of Y349, L351, L368, K392 and K409.

In some embodiments, the amino acid sequence of one polypeptide chain of the antibody constant region differs from the amino acid sequence of an IgG1 constant region at one or more positions selected from the group consisting of Y349, L351, L368, K392 and K409, and wherein the amino acid sequence of the other polypeptide chain of the antibody constant region differs from the amino acid sequence of an IgG1 constant region at one or more positions selected from the group consisting of L351, E356, T366 and D399.

In some embodiments, the amino acid sequence of one polypeptide chain of the antibody constant region differs from the amino acid sequence of an IgG1 constant region by K360E and K409W substitutions and wherein the amino acid sequence of the other polypeptide chain of the antibody constant region differs from the amino acid sequence of an IgG1 constant region by Q347R, D399V and F405T substitutions.

In some embodiments, the amino acid sequence of one polypeptide chain of the antibody constant region differs from the amino acid sequence of an IgG1 constant region by Q347R, D399V and F405T substitutions and wherein the amino acid sequence of the other polypeptide chain of the antibody constant region differs from the amino acid sequence of an IgG1 constant region by K360E and K409W substitutions.

In some embodiments, the amino acid sequence of one polypeptide chain of the antibody constant region differs from the amino acid sequence of an IgG1 constant region by a T366W substitutions and wherein the amino acid sequence of the other polypeptide chain of the antibody constant region differs from the amino acid sequence of an IgG1 constant region by T366S, T368A, and Y407V substitutions.

In some embodiments, the amino acid sequence of one polypeptide chain of the antibody constant region differs from the amino acid sequence of an IgG1 constant region by T366S, T368A, and Y407V substitutions and wherein the amino acid sequence of the other polypeptide chain of the antibody constant region differs from the amino acid sequence of an IgG1 constant region by a T366W substitution.

In some embodiments, the amino acid sequence of one polypeptide chain of the antibody constant region differs from the amino acid sequence of an IgG1 constant region by T350V, L351Y, F405A, and Y407V substitutions and wherein the amino acid sequence of the other polypeptide chain of the antibody constant region differs from the amino acid sequence of an IgG1 constant region by T350V, T366L, K392L, and T394W substitutions.

In some embodiments, the amino acid sequence of one polypeptide chain of the antibody constant region differs from the amino acid sequence of an IgG1 constant region by T350V, T366L, K392L, and T394W substitutions and wherein the amino acid sequence of the other polypeptide chain of the antibody constant region differs from the amino acid sequence of an IgG1 constant region by T350V, L351Y, F405A, and Y407V substitutions.

Alternatively, or additionally, the structural stability of a hetero-multimeric protein may be increased by introducing S354C on either of the first or second polypeptide chain, and Y349C on the opposing polypeptide chain, which forms an artificial disulfide bridge within the interface of the two polypeptides. In some embodiments, the amino acid sequence of one polypeptide chain of the antibody constant region differs from the amino acid sequence of an IgG1 constant region by an S354C substitution and wherein the amino acid sequence of the other polypeptide chain of the antibody constant region differs from the amino acid sequence of an IgG1 constant region by a Y349C substitution.

When selecting Fc substitutions, a skilled person would appreciate that the “one polypeptide chain” and “the other polypeptide chain” of an antibody constant region described above may correspond to the first antibody Fc sequence and the second antibody Fc sequence, respectively. Alternatively, the “one polypeptide chain” and “the other polypeptide chain” of an antibody constant region described above may correspond to the second antibody Fc sequence and the first antibody Fc sequence, respectively.

### Exemplary Multi-Specific Binding Proteins

Listed below are examples of TriNKETs comprising a HER2-binding scFv and an NKG2D-binding Fab each linked to an antibody constant region, wherein the antibody constant regions include mutations that enable heterodimerization of two Fc chains. The scFv comprises a heavy chain variable domain (VH) and a light chain variable domain (VL) derived from an anti-HER2 antibody (e.g., trastuzumab), and further comprises substitution of Cys for the amino acid residues at position 100 of VL and position 44 of VH, thereby facilitating formation of a disulfide bridge between the VH and VL of the scFv. The VL is linked N-terminal to the VH via a (G₄S)₄ linker (SEQ ID NO:203), and the VH is linked N-terminal to an Fc via an Ala-Ser linker. The Ala-Ser linker is included at the elbow hinge region sequence to balance between flexibility and optimal geometry. In certain embodiments, an additional sequence, Thr-Lys-Gly, can be added N-terminal or C-terminal to the Ala-Ser sequence at the hinge. As used herein to describe these exemplary TriNKETs, the Fc includes an antibody hinge, CH2, and CH3.

Accordingly, each of the TriNKETs described below comprises the following three polypeptide chains:

Chain A, comprising from N-terminus to C-terminus: VH of an NKG2D-binding Fab, CH1, and Fc;

Chain B, comprising from N-terminus to C-terminus: VL of a HER2-binding scFv, (G₄S)₄ linker (SEQ ID NO:203), VH of the HER2-binding scFv, Ala-Ser linker, and Fc; and

Chain C, comprising from N-terminus to C-terminus: VL of the NKG2D-binding Fab, and CL.

The amino acid sequences of the exemplary TriNKETs are summarized in Table 11.

In certain embodiments, the multi-specific binding protein of the present disclosure comprises a first polypeptide chain, a second polypeptide chain, and a third polypeptide chain, wherein the first, second, and third polypeptide chains comprise the amino acid sequences of Chain A, Chain B, and Chain C, respectively, of a TriNKET disclosed in Table 11. In certain embodiments, the first, second, and third polypeptide chains consist of the amino acid sequences of Chain A, Chain B, and Chain C, respectively, of a TriNKET disclosed in Table 11.

In an exemplary embodiment, the Fc domain linked to the NKG2D-binding Fab fragment comprises the mutations of Q347R, D399V, and F405T, and the Fc domain linked to the HER2 scFv comprises matching mutations K360E and K409W for forming a heterodimer. In another exemplary embodiment, the Fc domain linked to the NKG2D-binding Fab fragment comprises knob mutations T366S, L368A, and Y407V, and the Fc domain linked to the HER2-binding scFv comprises a “hole” mutation T366W. In an exemplary embodiment, the Fc domain linked to the NKG2D-binding Fab fragment includes an S354C substitution in the CH3 domain, which forms a disulfide bond with a Y349C substitution on the Fc linked to the HER2-binding scFv.

TABLE 11
Exemplary Multi-Specific Binding Proteins

| TriNKET | NKG2D | HER2 | Human | | | |
|---|---|---|---|---|---|---|
| Construct | Binding Fab | Binding scFv | IgG1 Fc | Chain A | Chain B | Chain C |
| A49-F3′-TriNKET- | A49 | Trastuzumab | EW-RVT | SEQ ID | SEQ ID | SEQ ID |
| Trastuzumab | | | | NO: 141 | NO: 140 | NO: 142 |
| A49-F3′-KiH-TriNKET- | A49 | Trastuzumab | KiH | SEQ ID | SEQ ID | SEQ ID |
| Trastuzumab | | | | NO: 147 | NO: 146 | NO: 142 |
| A49-F3′-TriNKET- | A49 | Pertuzumab | EW-RVT | SEQ ID | SEQ ID | SEQ ID |
| Pertuzumab | | | | NO: 141 | NO: 190 | NO: 142 |
| A49-F3′-KiH-TriNKET- | A49 | Pertuzumab | KiH | SEQ ID | SEQ ID | SEQ ID |
| Pertuzumab | | | | NO: 147 | NO: 191 | NO: 142 |
| A49-F3′-TriNKET- | A49 | MGAH22 | EW-RVT | SEQ ID | SEQ ID | SEQ ID |
| MGAH22 | | | | NO: 141 | NO: 192 | NO: 142 |
| A49-F3′-KiH-TriNKET- | A49 | MGAH22 | KiH | SEQ ID | SEQ ID | SEQ ID |
| MGAH22 | | | | NO: 147 | NO: 193 | NO: 142 |
| A49MI-F3′-TriNKET- | A49MI | Trastuzumab | EW-RVT | SEQ ID | SEQ ID | SEQ ID |
| Trastuzumab | | | | NO: 145 | NO: 140 | NO: 142 |
| A49MI-F3′-KiH-TriNKET- | A49MI | Trastuzumab | KiH | SEQ ID | SEQ ID | SEQ ID |
| Trastuzumab | | | | NO: 194 | NO: 146 | NO: 142 |
| A49MI-F3′-TriNKET- | A49MI | Pertuzumab | EW-RVT | SEQ ID | SEQ ID | SEQ ID |
| Pertuzumab | | | | NO: 145 | NO: 190 | NO: 142 |
| A49MI-F3′-KiH-TriNKET- | A49MI | Pertuzumab | KiH | SEQ ID | SEQ ID | SEQ ID |
| Pertuzumab | | | | NO: 194 | NO: 191 | NO: 142 |
| A49MI-F3′-TriNKET- | A49MI | MGAH22 | EW-RVT | SEQ ID | SEQ ID | SEQ ID |
| MGAH22 | | | | NO: 145 | NO: 192 | NO: 142 |
| A49MI-F3′-KiH-TriNKET- | A49MI | MGAH22 | KiH | SEQ ID | SEQ ID | SEQ ID |
| MGAH22 | | | | NO: 194 | NO: 193 | NO: 142 |
| A44-F3′-TriNKET- | A44 | Trastuzumab | EW-RVT | SEQ ID | SEQ ID | SEQ ID |
| Trastuzumab | | | | NO: 155 | NO: 140 | NO: 149 |
| A44-F3′-KiH-TriNKET- | A44 | Trastuzumab | KiH | SEQ ID | SEQ ID | SEQ ID |
| Trastuzumab | | | | NO: 148 | NO: 146 | NO: 149 |
| A44-F3′-TriNKET- | A44 | Pertuzumab | EW-RVT | SEQ ID | SEQ ID | SEQ ID |
| Pertuzumab | | | | NO: 155 | NO: 190 | NO: 149 |
| A44-F3′-KiH-TriNKET- | A44 | Pertuzumab | KiH | SEQ ID | SEQ ID | SEQ ID |
| Pertuzumab | | | | NO: 148 | NO: 191 | NO: 149 |
| A44-F3′-TriNKET- | A44 | MGAH22 | EW-RVT | SEQ ID | SEQ ID | SEQ ID |
| MGAH22 | | | | NO: 155 | NO: 192 | NO: 149 |
| A44-F3′-KiH-TriNKET- | A44 | MGAH22 | KiH | SEQ ID | SEQ ID | SEQ ID |
| MGAH22 | | | | NO: 148 | NO: 193 | NO: 149 |

Specific TriNKETs and their polypeptide chains are described in more detail below. In the amino acid sequences, (G₄S)₄ (SEQ ID NO:203) and Ala-Ser linkers are bold-underlined; Cys residues in the scFv that form disulfide bridges are bold-italic-underlined; Fc heterodimerization mutations are bold-underlined; and CDR sequences under Kabat are underlined.

For example, a TriNKET of the present disclosure is A49-F3′-TriNKET-Trastuzumab. A49-F3′-TriNKET-Trastuzumab includes a single-chain variable fragment (scFv) (SEQ ID NO:139) derived from trastuzumab that binds HER2, linked via a hinge comprising Ala-Ser to an Fc domain; and an NKG2D-binding Fab fragment derived from A49 including a heavy chain portion comprising a heavy chain variable domain (SEQ ID NO:94) and a CH1 domain, and a light chain portion comprising a light chain variable domain (SEQ ID NO:98) and a light chain constant domain, wherein the heavy chain variable domain is connected to the CH1 domain, and the CH1 domain is connected to the Fc domain A49-F3′-TriNKET-Trastuzumab includes three polypeptides having the sequences of SEQ ID NO:140, SEQ ID NO:141, and SEQ ID NO:142.

SEQ ID NO:140 represents the full sequence of the HER2-binding scFv linked to an Fc domain via a hinge comprising Ala-Ser (scFv-Fc). The Fc domain linked to the scFv includes Q347R, D399V, and F405T substitutions for heterodimerization and an S354C substitution for forming a disulfide bond with a Y349C substitution in SEQ ID NO:141 as described below. The scFv (SEQ ID NO:139) includes a heavy chain variable domain of trastuzumab connected to the N-terminus of a light chain variable domain of trastuzumab via a (G₄S)₄ linker (SEQ ID NO:203), the scFv represented as VL-(G₄S)₄-VH (“(G₄S)₄” is represented by SEQ ID NO:203 or SEQ ID NO:143). The heavy and the light variable domains of the scFv are also connected through a disulfide bridge between C100 of VL and C44 of VH, as a result of Q100C and G44C substitutions in the VL and VH, respectively.

Trastuzumab scFv
(SEQ ID NO: 139)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFG [symbol]
GTKVEIKGGGGSGGGGSGGGGSGGGGSEVQLVESGGGLVQPGGSLRLSCA
ASGFNIKDTYIHWVRQAPGK [symbol] LEWVARIYPTNGYTRYADSVKGRFTISAD
TSKNTAYLQMNSLRAEDTAVYYCSRWGGDGFYAMDYWGQGTLVTVSS
Trastuzumab scFv-Fc (RVT)
(SEQ ID NO: 140)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFG [symbol]
GTKVEIKGGGGSGGGGSGGGGSGGGGSEVQLVESGGGLVQPGGSLRLSCA
ASGFNIKDTYIHWVRQAPGK [symbol] LEWVARIYPTNGYTRYADSVKGRFTISAD
TSKNTAYLQMNSLRAEDTAVYYCSRWGGDGFYAMDYWGQGTLVTVSSASD
KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPRVYTLPP [symbol] RDELTKNQVSLTCLVKG
FYPSDIAVEWESNGQPENNYKTTPPVLVSDGSFTLYSKLTVDKSRWQQGN
VFSCSVMHEALHNHYTQKSLSLSPG

SEQ ID NO:141 represents the heavy chain portion of the Fab fragment, which comprises a heavy chain variable domain (SEQ ID NO:94) of an NKG2D-binding site and a CH1 domain, connected to an Fc domain. The Fc domain in SEQ ID NO:141 includes a Y349C substitution in the CH3 domain, which forms a disulfide bond with an S354C substitution on the Fc linked to the HER2-binding scFv (SEQ ID NO:140). In SEQ ID NO:141, the Fc domain also includes K360E and K409W substitutions for heterodimerization with the Fc in SEQ ID NO:140.

A49 VH
(SEQ ID NO: 94)
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
PMGAAAGWFDPWGQGTLVTVSS
A49 VH-CH1-Fc (EW)
(SEQ ID NO: 141)
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
PMGAAAGWFDPWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQV [symbol] TLPPSRDELTENQVSLTCLVKGFYPSDIAVEWESNGQPENNYKTTP
PVLDSDGSFFLYSWLTVDKSRWQQGNVFSCSVMHEALHNHYTQKSLSLSP
G

SEQ ID NO:142 represents the light chain portion of the Fab fragment comprising a light chain variable domain (SEQ ID NO:98) of an NKG2D-binding site and a light chain constant domain.

A49 VL
(SEQ ID NO: 98)
DIQMTQSPSSVSASVGDRVTITCRASQGISSWLAWYQQKPGKAPKLLIYA
ASSLQSGVPSRFSGSGSGTDFTLTISSLQPEDFATYYCQQGVSFPRTFGG
GTKVEIK
A49 VL-LC
(SEQ ID NO: 142)
DIQMTQSPSSVSASVGDRVTITCRASQGISSWLAWYQQKPGKAPKLLIYA
ASSLQSGVPSRFSGSGSGTDFTLTISSLQPEDFATYYCQQGVSFPRTFGG
GTKVEIKRTVAAPSPPSDEQLKSGTASVVCLLNNFYPREAKVQWKVDNAL
QSGNSQESVTEQDSKDSTYSLSSTLTLSKADYEKHKVYACEVTHQGLSSP
VTKSFNRGEC

Another TriNKET of the present disclosure is A49MI-F3′-TriNKET-Trastuzumab. A49MI-F3′-TriNKET-Trastuzumab includes the same Her2-binding scFv (SEQ ID NO:139) as in A49-F3′-TriNKET-Trastuzumab linked via a hinge comprising Ala-Ser to an Fc domain; and an NKG2D-binding Fab fragment derived from A49MI including a heavy chain portion comprising a heavy chain variable domain (SEQ ID NO:144) and a CH1 domain, and a light chain portion comprising a light chain variable domain (SEQ ID NO:98) and a light chain constant domain, wherein the heavy chain variable domain is connected to the CH1 domain, and the CH1 domain is connected to the Fc domain. A49MI-F3′-TriNKET-Trastuzumab includes three polypeptides having the sequences of SEQ ID NO:140 (as in A49-F3′-TriNKET-Trastuzumab), SEQ ID NO:145, and SEQ ID NO:142 (as in A49-F3′-TriNKET-Trastuzumab).

SEQ ID NO:145 represents a heavy chain portion of the Fab fragment, which comprises a heavy chain variable domain (SEQ ID NO:144) of an NKG2D-binding site and a CH1 domain, connected to an Fc domain. In SEQ ID NO:144, wherein a methionine in the CDR3 of SEQ ID NO:94 has been substituted by isoleucine (M→I substitution; shown within a third bracket [] in SEQ ID NO:144 and SEQ ID NO:145). The Fc domain in SEQ ID NO:145 includes a Y349C substitution in the CH3 domain, which forms a disulfide bond with an S354C substitution in the Fc linked to the HER2-binding scFv (SEQ ID NO:140). In SEQ ID NO:145, the Fc domain also includes K360E and K409W substitutions.

A49MI VH
(SEQ ID NO: 144)
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
P[I]GAAAGWFDPWGQGTLVTVSS
A49MI VH-CH1-Fc (EW)
(SEQ ID NO: 145)
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
P[I]GAAAGWFDPWGQGTLVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQV [symbol] TLPPSRDELTENQVSLTCLVKGFYPSDIAVEWESNGQPENNYKT
TPPVLDSDGSFFLYSWLTVDKSRWQQGNVFSCSVMHEALHNHYTQKSLSL
SPG

Another TriNKET of the present disclosure is A49-F3′-KiH-TriNKET-Trastuzumab. KiH refers to the knobs-into-holes (KiH) Fc technology, which involves engineering of the CH3 domains to create either a “knob” or a “hole” in each heavy chain to promote heterodimerization. The concept behind the KiH Fc technology was to introduce a “knob” in one CH3 domain (CH3A) by substitution of a small residue with a bulky one (e.g., T366W_{CH3A} in EU numbering). To accommodate the “knob,” a complementary “hole” surface was created on the other CH3 domain (CH3B) by replacing the closest neighboring residues to the knob with smaller ones (e.g., T366S/L368A/Y407V_{CH3B}). The “hole” mutation was optimized by structure-guided phage library screening (Atwell S, Ridgway J B, Wells J A, Carter P., Stable heterodimers from remodeling the domain interface of a homodimer using a phage display library, J. Mol. Biol. (1997) 270(1):26-35). X-ray crystal structures of KiH Fc variants (Elliott J M, Ultsch M, Lee J, Tong R, Takeda K, Spiess C, et al., Antiparallel conformation of knob and hole aglycosylated half-antibody homodimers is mediated by a CH2-CH3 hydrophobic interaction. J. Mol. Biol. (2014) 426(9):1947-57; Mimoto F, Kadono S, Katada H, Igawa T, Kamikawa T, Hattori K. Crystal structure of a novel asymmetrically engineered Fc variant with improved affinity for FcγRs. Mol. Immunol. (2014) 58(1):132-8) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

A49-F3′-KiH-TriNKET-Trastuzumab includes the same Her2-binding scFv (SEQ ID NO:139) as in A49-F3′-TriNKET-Trastuzumab linked via a hinge comprising Ala-Ser to an Fc domain comprising the “hole” substitutions of T366S, L368A, and Y407V; and the same NKG2D-binding Fab fragment as in A49-F3′-TriNKET-Trastuzumab, the CH1 domain of which is connected to an Fc domain comprising the “knob” substitution of T366W. A49-F3′-KiH-TriNKET-Trastuzumab includes three polypeptides having the sequences of SEQ ID NO:146, SEQ ID NO:147, and SEQ ID NO:142 (as in A49-F3′-TriNKET-Trastuzumab).

SEQ ID NO:146 represents the full sequence of the HER2-binding scFv (SEQ ID NO:139) linked to an Fc domain via a hinge comprising Ala-Ser (scFv-Fc). The Fc domain linked to the scFv includes T366S, L368A, and Y407V substitutions for heterodimerization and an S354C substitution for forming a disulfide bond with a Y349C substitution in SEQ ID NO:147 as described below.

Trastuzumab scFv-Fc (KiH)
(SEQ ID NO: 146)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFG [symbol]
GTKVEIKGGGGSGGGGSGGGGSGGGGSEVQLVESGGGLVQPGGSLRLSCA
ASGFNIKDTYIHWVRQAPGK [symbol] LEWVARIYPTNGYTRYADSVKGRFTISAD
TSKNTAYLQMNSLRAEDTAVYYCSRWGGDGFYAMDYWGQGTLVTVSSASD
KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQV [symbol] TLPPSRDELTKNQVSLSCAVKG
FYPSDIAVEWESNGQPENNYKTTPPVLDSDGSFFLVSKLTVDKSRWQQGN
VFSCSVMHEALHNHYTQKSLSLSPG

SEQ ID NO:147 represents the heavy chain portion of a Fab fragment, which comprises a heavy chain variable domain (SEQ ID NO:94) of an NKG2D-binding site derived from A49 and a CH1 domain, connected to an Fc domain. The Fc domain in SEQ ID NO:147 includes an S354C substitution, which forms a disulfide bond with a Y349C substitution in the CH3 domain of the Fc linked to the HER2-binding scFv (SEQ ID NO:146). In SEQ ID NO:147, the Fc domain also includes a T366W substitution.

A49 VH-CH1-Fc (KiH)
(SEQ ID NO: 147)
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
PMGAAAGWFDPWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPP [symbol] RDELTKNQVSLWCLVKGFYPSDIAVEWESNGQPENNYKTTP
PVLDSDGSFFLYSKLTVDKSRWQQGNVFSCSVMHEALHNHYTQKSLSLSP
G

Another TriNKET of the present disclosure is A49MI-F3′-KiH-TriNKET-Trastuzumab. A49MI-F3′-KiH-TriNKET-Trastuzumab includes the same Her2-binding scFv (SEQ ID NO:139) as in A49-F3′-TriNKET-Trastuzumab linked via a hinge comprising Ala-Ser to an Fc domain comprising the “hole” substitutions of T366S, L368A, and Y407V; and the same NKG2D-binding Fab fragment as in A49MI-F3′-TriNKET-Trastuzumab, the CH1 domain of which is connected to an Fc domain comprising the “knob” substitution of T366W. A49MI-F3′-KiH-TriNKET-Trastuzumab includes three polypeptides having the sequences of SEQ ID NO:146 (as in A49-F3′-KiH-TriNKET-Trastuzumab), SEQ ID NO:194, and SEQ ID NO:142 (as in A49-F3′-TriNKET-Trastuzumab).

SEQ ID NO:194 represents the heavy chain portion of a Fab fragment, which comprises a heavy chain variable domain (SEQ ID NO:144) of an NKG2D-binding site derived from A49MI and a CH1 domain, connected to an Fc domain The Fc domain in SEQ ID NO:194 includes an S354C substitution, which forms a disulfide bond with a Y349C substitution in the CH3 domain of the Fc linked to the HER2-binding scFv (SEQ ID NO:146). In SEQ ID NO:194, the Fc domain also includes a T366W substitution.

A49MI VH-CH1-Fc (KiH)
(SEQ ID NO: 194)
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
PIGAAAGWFDPWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPP [symbol] RDELTKNQVSLWCLVKGFYPSDIAVEWESNGQPENNYKTTP
PVLDSDGSFFLYSKLTVDKSRWQQGNVFSCSVMHEALHNHYTQKSLSLSP
G

Another exemplary TriNKET of the present disclosure is A44-F3′-TriNKET-Trastuzumab. A44-F3′-TriNKET-Trastuzumab includes the same Her2-binding scFv (SEQ ID NO:139) as in A49-F3′-TriNKET-Trastuzumab linked via a hinge comprising Ala-Ser to an Fc domain; and an NKG2D-binding Fab fragment derived from A44 including a heavy chain portion comprising a heavy chain variable domain (SEQ ID NO:86) and a CH1 domain, and a light chain portion comprising a light chain variable domain (SEQ ID NO:90) and a light chain constant domain, wherein the heavy chain variable domain is connected to the CH1 domain, and the CH1 domain is connected to the Fc domain A44-F3′-TriNKET-Trastuzumab includes three polypeptides having the sequences of SEQ ID NO:140 (as in A49-F3′-TriNKET-Trastuzumab), SEQ ID NO:155, and SEQ ID NO:149.

SEQ ID NO:155 represents a heavy chain variable domain (SEQ ID NO:86) of an NKG2D-binding site derived from A44, connected to an Fc domain. The Fc domain in SEQ ID NO:155 includes a Y349C substitution in the CH3 domain, which forms a disulfide bond with an S354C substitution on the Fc linked to the HER2-binding scFv (SEQ ID NO:140). In SEQ ID NO:155, the Fc domain also includes K360E and K409W substitutions.

A44 VH
(SEQ ID NO: 86)
EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKDG
GYYDSGAGDYWGQGTLVTVSS
A44 VH-CH1-Fc (EW)
(SEQ ID NO: 155)
EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKDG
GYYDSGAGDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QV [symbol] TLPPSRDELTENQVSLTCLVKGFYPSDIAVEWESNGQPENNYKTTPP
VLDSDGSFFLYSWLTVDKSRWQQGNVFSCSVMHEALHNHYTQKSLSLSPG

SEQ ID NO:149 represents the light chain portion of the Fab fragment comprising a light chain variable domain (SEQ ID NO:90) of an NKG2D-binding site and a light chain constant domain.

A44 VL
(SEQ ID NO: 90)
DIQMTQSPSSVSASVGDRVTITCRASQGIDSWLAWYQQKPGKAPKLLIYA
ASSLQSGVPSRFSGSGSGTDFTLTISSLQPEDFATYYCQQGVSYPRTFGG
GTKVEIK
A44 VL-CL
(SEQ ID NO: 149)
DIQMTQSPSSVSASVGDRVTITCRASQGIDSWLAWYQQKPGKAPKLLIYA
ASSLQSGVPSRFSGSGSGTDFTLTISSLQPEDFATYYCQQGVSYPRTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Another exemplary TriNKET of the present disclosure is A44-F3′-KiH-TriNKET-Trastuzumab. A44-F3′-KiH-TriNKET-Trastuzumab includes the same Her2-binding scFv (SEQ ID NO:139) as in A49-F3′-TriNKET-Trastuzumab linked via a hinge comprising Ala-Ser to an Fc domain comprising the “hole” substitutions of T366S, L368A, and Y407V; and the same NKG2D-binding Fab fragment as in A44-F3′-TriNKET-Trastuzumab, the CH1 domain of which is connected to an Fc domain comprising the “knob” substitution of T366W. A44-F3′-KiH-TriNKET-Trastuzumab includes three polypeptides having the sequences of SEQ ID NO:146 (as in A49-F3′-KiH-TriNKET-Trastuzumab), SEQ ID NO:148, and SEQ ID NO:149 (as in A44-F3′-TriNKET-Trastuzumab).

SEQ ID NO:148 represents a heavy chain variable domain (SEQ ID NO:86) of an NKG2D-binding site derived from A44, connected to an Fc domain. The Fc domain in SEQ ID NO:148 includes a Y349C substitution in the CH3 domain, which forms a disulfide bond with an S354C substitution on the Fc linked to the HER2-binding scFv (SEQ ID NO:146). In SEQ ID NO:148, the Fc domain also includes a T366W substitution.

A44 VH-CH1-Fc (KiH)
(SEQ ID NO: 148)
EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKDG
GYYDSGAGDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPP [symbol] RDELTKNQVSLWCLVKGFYPSDIAVEWESNGQPENNYKTTPP
VLDSDGSFFLYSKLTVDKSRWQQGNVFSCSVMHEALHNHYTQKSLSLSPG

Another TriNKET of the present disclosure is A49-F3′-TriNKET-Pertuzumab. A49-F3′-TriNKET-Pertuzumab includes an scFv (SEQ ID NO:189) derived from pertuzumab that binds HER2, linked via a hinge comprising Ala-Ser to an Fc domain; and the same NKG2D-binding Fab fragment as in A49-F3′-TriNKET-Trastuzumab, the CH1 domain of which is connected to an Fc domain. The Fc domain linked to the scFv includes Q347R, D399V, and F405T substitutions, and the Fc domain linked to the Fab fragment includes K360E and K409W substitutions. A49-F3′-TriNKET-Pertuzumab includes three polypeptides, having the sequences of SEQ ID NO:190, SEQ ID NO:141 (as in A49-F3′-TriNKET-Trastuzumab), and SEQ ID NO:142 (as in A49-F3′-TriNKET-Trastuzumab).

SEQ ID NO:190 represents the full sequence of the HER2-binding scFv linked to an Fc domain via a hinge comprising Ala-Ser (scFv-Fc). The Fc domain linked to the scFv includes Q347R, D399V, and F405T substitutions for heterodimerization and an S354C substitution for forming a disulfide bond with a Y349C substitution in SEQ ID NO:141 as described above. The scFv (SEQ ID NO:189) includes a heavy chain variable domain of pertuzumab connected to the N-terminus of a light chain variable domain of pertuzumab via a (G₄S)₄ linker (SEQ ID NO:203), the scFv represented as VL-(G₄S)₄-VH (“(G₄S)₄” is represented by SEQ ID NO:203 or SEQ ID NO:143). The heavy and the light variable domains of the scFv are also connected through a disulfide bridge between C100 of VL and C44 of VH, as a result of Q100C and G44C substitutions in the VL and VH, respectively.

Pertuzumab scFv
(SEQ ID NO: 189)
DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFG [symbol]
GTKVEIKRGGGGSGGGGSGGGGSGGGGSEVQLVESGGGLVQPGGSLRLSC
AASGFTFTDYTMDWVRQAPGK [symbol] LEWVADVNPNSGGSIYNQRFKGRFTLSV
DRSKNTLYLQMNSLRAEDTAVYYCARNLGPSFYPDYWGQGTLVTVSSA
Pertuzumab scFv-Fc
(SEQ ID NO: 190)
DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFG [symbol]
GTKVEIKRGGGGSGGGGSGGGGSGGGGSEVQLVESGGGLVQPGGSLRLSC
AASGFTFTDYTMDWVRQAPGK [symbol] LEWVADVNPNSGGSIYNQRFKGRFTLSV
DRSKNTLYLQMNSLRAEDTAVYYCARNLGPSFYPDYWGQGTLVTVSSAAS
DKTHTCPP [symbol] PAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPRVYTLPP [symbol] RDELTKNQVSLTCLVK
GFYPSDIAVEWESNGQPENNYKTTPPVLVSDGSFTLYSKLTVDKSRWQQG
NVFSCSVMHEALHNHYTQKSLSLSPG

Another exemplary TriNKET of the present disclosure is A49MI-F3′-TriNKET-Pertuzumab. A49MI-F3′-TriNKET-Pertuzumab includes the same Her2-binding scFv (SEQ ID NO:189) as in A49-F3′-TriNKET-Pertuzumab linked via a hinge comprising Ala-Ser to an Fc domain; and the same NKG2D-binding Fab fragment as in A49MI-F3′-TriNKET-Trastuzumab, the CH1 domain of which is connected to an Fc domain. The Fc domain linked to the scFv includes Q347R, D399V, and F405T substitutions, and the Fc domain linked to the Fab fragment includes K360E and K409W substitutions. A49MI-F3′-TriNKET-Pertuzumab includes three polypeptides having the sequences of SEQ ID NO:190 (as in A49-F3′-KiH-TriNKET-Pertuzumab), SEQ ID NO:145 (as in A49MI-F3′-TriNKET-Trastuzumab), and SEQ ID NO:142 (as in A49-F3′-TriNKET-Trastuzumab).

Another exemplary TriNKET of the present disclosure is A49-F3′-KiH-TriNKET-Pertuzumab. A49-F3′-KiH-TriNKET-Pertuzumab includes the same Her2-binding scFv (SEQ ID NO:189) as in A49-F3′-TriNKET-Pertuzumab linked via a hinge comprising Ala-Ser to an Fc domain; and the same NKG2D-binding Fab fragment as in A49-F3′-TriNKET-Trastuzumab, the CH1 domain of which is connected to an Fc domain. The Fc domain linked to the scFv includes the “hole” substitutions of T366S, L368A, and Y407V, and the Fc domain linked to the Fab fragment includes the “knob” substitution of T366W. A49-F3′-KiH-TriNKET-Pertuzumab includes three polypeptides, having the sequences of SEQ ID NO:191, SEQ ID NO:147 (as in A49-F3′-KiH-TriNKET-Trastuzumab), and SEQ ID NO:142 (as in A49-F3′-TriNKET-Trastuzumab).

SEQ ID NO:191 represents the full sequence of the HER2-binding scFv (SEQ ID NO:189) linked to an Fc domain via a hinge comprising Ala-Ser (scFv-Fc). The Fc domain linked to the scFv includes T366S, L368A, and Y407V substitutions for heterodimerization and an S354C substitution for forming a disulfide bond with a Y349C substitution in SEQ ID NO:191 as described above.

Pertuzumab scFv-Fc (KiH)
(SEQ ID NO: 191)
DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFG [symbol]
GTKVEIKRGGGGSGGGGSGGGGSGGGGSEVQLVESGGGLVQPGGSLRLSC
AASGFTFTDYTMDWVRQAPGK [symbol] LEWVADVNPNSGGSIYNQRFKGRFTLSV
DRSKNTLYLQMNSLRAEDTAVYYCARNLGPSFYFDYWGQGTLVTVSSAAS
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQV [symbol] TLPPSRDELTKNQVSLSCAVK
GFYPSDIAVEWESNGQPENNYKTTPPVLDSDGSFFLVSKLTVDKSRWQQG
NVFSCSVMHEALHNHYTQKSLSLSPG

Another exemplary TriNKET of the present disclosure is A49MI-F3′-KiH-TriNKET-Pertuzumab. A49MI-F3′-KiH-TriNKET-Pertuzumab includes the same Her2-binding scFv (SEQ ID NO:189) as in A49-F3′-TriNKET-Pertuzumab linked via a hinge comprising Ala-Ser to an Fc domain; and the same NKG2D-binding Fab fragment as in A49MI-F3′-TriNKET-Trastuzumab, the CH1 domain of which is connected to an Fc domain. The Fc domain linked to the scFv includes the “hole” substitutions of T366S, L368A, and Y407V, and the Fc domain linked to the Fab fragment includes the “knob” substitution of T366W. A49MI-F3′-KiH-TriNKET-Pertuzumab includes three polypeptides having the sequences of SEQ ID NO:191 (as in A49-F3′-KiH-TriNKET-Pertuzumab), SEQ ID NO:194 (as in A49MI-F3′-KiH-TriNKET-Trastuzumab), and SEQ ID NO:142 (as in A49-F3′-TriNKET-Trastuzumab).

Another exemplary TriNKET of the present disclosure is A44-F3′-TriNKET-Pertuzumab. A44-F3′-TriNKET-Pertuzumab includes the same Her2-binding scFv (SEQ ID NO:189) as in A49-F3′-TriNKET-Pertuzumab linked via a hinge comprising Ala-Ser to an Fc domain; and the same NKG2D-binding Fab fragment as in A44-F3′-TriNKET-Trastuzumab, the CH1 domain of which is connected to an Fc domain. The Fc domain linked to the scFv includes Q347R, D399V, and F405T substitutions, and the Fc domain linked to the Fab fragment includes K360E and K409W substitutions. A44-F3′-TriNKET-Pertuzumab includes three polypeptides having the sequences of SEQ ID NO:190 (as in A49-F3′-KiH-TriNKET-Pertuzumab), SEQ ID NO:155 (as in A44-F3′-TriNKET-Trastuzumab), and SEQ ID NO:149 (as in A44-F3′-TriNKET-Trastuzumab).

Another exemplary TriNKET of the present disclosure is A44-F3′-KiH-TriNKET-Pertuzumab. A44-F3′-KiH-TriNKET-Pertuzumab includes the same Her2-binding scFv (SEQ ID NO:189) as in A49-F3′-TriNKET-Pertuzumab linked via a hinge comprising Ala-Ser to an Fc domain; and the same NKG2D-binding Fab fragment as in A44-F3′-TriNKET-Trastuzumab, the CH1 domain of which is connected to an Fc domain. The Fc domain linked to the scFv includes the “hole” substitutions of T366S, L368A, and Y407V, and the Fc domain linked to the Fab fragment includes the “knob” substitution of T366W. A44-F3′-KiH-TriNKET-Pertuzumab includes three polypeptides having the sequences of SEQ ID NO:191 (as in A49-F3′-KiH-TriNKET-Pertuzumab), SEQ ID NO:148 (as in A44-F3′-KiH-TriNKET-Trastuzumab), and SEQ ID NO:149 (as in A44-F3′-TriNKET-Trastuzumab).

Another TriNKET of the present disclosure is A49-F3′-TriNKET-MGAH22. A49-F3′-TriNKET-MGAH22 includes an scFv (SEQ ID NO:171) derived from MGAH22 that binds HER2, linked via a hinge comprising Ala-Ser to an Fc domain; and the same NKG2D-binding Fab fragment as in A49-F3′-TriNKET-Trastuzumab, the CH1 domain of which is connected to an Fc domain. The Fc domain linked to the scFv includes Q347R, D399V, and F405T substitutions, and the Fc domain linked to the Fab fragment includes K360E and K409W substitutions. A49-F3′-TriNKET-MGAH22 includes three polypeptides having the sequences of SEQ ID NO:192, SEQ ID NO:141 (as in A49-F3′-TriNKET-Trastuzumab), and SEQ ID NO:142 (as in A49-F3′-TriNKET-Trastuzumab).

SEQ ID NO:192 represents the full sequence of the HER2-binding scFv linked to an Fc domain via a hinge comprising Ala-Ser (scFv-Fc). The Fc domain linked to the scFv includes Q347R, D399V, and F405T substitutions for heterodimerization and an S354C substitution for forming a disulfide bond with a Y349C substitution in SEQ ID NO:141 as described above. The scFv (SEQ ID NO:171) includes a heavy chain variable domain of pertuzumab connected to the N-terminus of a light chain variable domain of pertuzumab via a (G₄S)₄ linker (SEQ ID NO:203), the scFv represented as VL-(G₄S)₄-VH (“(G₄S)₄” is represented by SEQ ID NO:203 or SEQ ID NO:143). The heavy and the light variable domains of the scFv are also connected through a disulfide bridge between C100 of VL and C44 of VH, as a result of G100C and G44C substitutions in the VL and VH, respectively.

MGAH22 scFv
(SEQ ID NO: 171)
DIVMTQSHKFMSTSVGDRVSITCKASQDVNTAVAWYQQKPGHSPKLLIYS
ASFRYTGVPDRFTGSRSGTDFTFTISSVQAEDLAVYYCQQHYTTPPTFG [symbol]
GTKVEIKRGGGGSGGGGSGGGGSGGGGSQVQLQQSGPELVKPGASLKLSC
TASGFNIKDTYIHWVKQRPEQ [symbol] LEWIGRIYPTNGYTRYDPKFQDKATITA
DTSSNTAYLQVSRLTSEDTAVYYCSRWGGDGFYAMDYWGQGASVTVSSA
MGAH22 scFv-Fc
(SEQ ID NO: 192)
DIVMTQSHKFMSTSVGDRVSITCKASQDVNTAVAWYQQKPGHSPKLLIYS
ASFRYTGVPDRFTGSRSGTDFTFTISSVQAEDLAVYYCQQHYTTPPTFG [symbol]
GTKVEIKRGGGGSGGGGSGGGGSGGGGSQVQLQQSGPELVKPGASLKLSC
TASGFNIKDTYIHWVKQRPEQ [symbol] LEWIGRIYPTNGYTRYDPKFQDKATITA
DTSSNTAYLQVSRLTSEDTAVYYCSRWGGDGFYAMDYWGQGASVTVSSAA
SDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPRVYTLPP [symbol] RDELTKNQVSLTCLV
KGFYPSDIAVEWESNGQPENNYKTTPPVLVSDGSFTLYSKLTVDKSRWQQ
GNVFSCSVMHEALHNHYTQKSLSLSPG

Another TriNKET of the present disclosure is A49MI-F3′-TriNKET-MGAH22. A49MI-F3′-TriNKET-MGAH22 includes the same Her2-binding scFv (SEQ ID NO:171) as in A49-F3′-TriNKET-MGAH22 linked via a hinge comprising Ala-Ser to an Fc domain; and the same NKG2D-binding Fab fragment as in A49MI-F3′-TriNKET-Trastuzumab, the CH1 domain of which is connected to an Fc domain. The Fc domain linked to the scFv includes Q347R, D399V, and F405T substitutions, and the Fc domain linked to the Fab fragment includes K360E and K409W substitutions. A49MI-F3′-KiH-TriNKET-MGAH22 includes three polypeptides, having the sequences of SEQ ID NO:192 (as in A49-F3′-TriNKET-MGAH22), SEQ ID NO:145 (as in A49MI-F3′-TriNKET-Trastuzumab), and SEQ ID NO:142 (as in A49-F3′-TriNKET-Trastuzumab).

Another TriNKET of the present disclosure is A49-F3′-KiH-TriNKET-MGAH22. A49-F3′-KiH-TriNKET-MGAH22 includes the same Her2-binding scFv (SEQ ID NO:171) as in A49-F3′-TriNKET-MGAH22 linked via a hinge comprising Ala-Ser to an Fc domain; and the same NKG2D-binding Fab fragment as in A49-F3′-TriNKET-Trastuzumab, the CH1 domain of which is connected to an Fc domain. The Fc domain linked to the scFv includes the “hole” substitutions of T366S, L368A, and Y407V, and the Fc domain linked to the Fab fragment includes the “knob” substitution of T366W. A49-F3′-KiH-TriNKET-MGAH22 includes three polypeptides having the sequences of SEQ ID NO:193, SEQ ID NO:147 (as in A49-F3′-KiH-TriNKET-Trastuzumab), and SEQ ID NO:142 (as in A49-F3′-TriNKET-Trastuzumab).

SEQ ID NO:193 represents the full sequence of the HER2-binding scFv (SEQ ID NO:171) linked to an Fc domain via a hinge comprising Ala-Ser (scFv-Fc). The Fc domain linked to the scFv includes T366S, L368A, and Y407V substitutions for heterodimerization and an S354C substitution for forming a disulfide bond with a Y349C substitution in SEQ ID NO:147 as described above.

MGAH22 scFv-Fc (KiH)
(SEQ ID NO: 193)
DIVMTQSHKFMSTSVGDRVSITCKASQDVNTAVAWYQQKPGHSPKLLIYS
ASFRYTGVPDRFTGSRSGTDFTFTISSVQAEDLAVYYCQQHYTTPPTFG [symbol]
GTKVEIKRGGGGSGGGGSGGGGSGGGGSQVQLQQSGPELVKPGASLKLSC
TASGFNIKDTYIHWVKQRPEQ [symbol] LEWIGRIYPTNGYTRYDPKFQDKATITA
DTSSNTAYLQVSRLTSEDTAVYYCSRWGGDGFYAMDYWGQGASVTVSSAA
SDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQV [symbol] TLPPSRDELTKNQVSLSCAV
KGFYPSDIAVEWESNGQPENNYKTTPPVLDSDGSFFLVSKLTVDKSRWQQ
GNVFSCSVMHEALHNHYTQKSLSLSPG

Another exemplary TriNKET of the present disclosure is A49MI-F3′-KiH-TriNKET-MGAH22. A49MI-F3′-KiH-TriNKET-MGAH22 includes the same Her2-binding scFv (SEQ ID NO:171) as in A49-F3′-TriNKET-MGAH22 linked via a hinge comprising Ala-Ser to an Fc domain; and the same NKG2D-binding Fab fragment as in A49MI-F3′-TriNKET-Trastuzumab, the CH1 domain of which is connected to an Fc domain. The Fc domain linked to the scFv includes the “hole” substitutions of T366S, L368A, and Y407V, and the Fc domain linked to the Fab fragment includes the “knob” substitution of T366W. A49MI-F3′-KiH-TriNKET-MGAH22 includes three polypeptides having the sequences of SEQ ID NO:193 (as in A49-F3′-KiH-TriNKET-MGAH22), SEQ ID NO:194 (as in A49MI-F3′-KiH-TriNKET-Trastuzumab), and SEQ ID NO:142 (as in A49-F3′-TriNKET-Trastuzumab).

Another exemplary TriNKET of the present disclosure is A44-F3′-TriNKET-MGAH22. A44-F3′-TriNKET-MGAH22 includes the same Her2-binding scFv (SEQ ID NO:171) as in A49-F3′-TriNKET-MGAH22 linked via a hinge comprising Ala-Ser to an Fc domain; and the same NKG2D-binding Fab fragment as in A44-F3′-TriNKET-Trastuzumab, the CH1 domain of which is connected to an Fc domain. The Fc domain linked to the scFv includes Q347R, D399V, and F405T substitutions, and the Fc domain linked to the Fab fragment includes K360E and K409W substitutions. A44-F3′-TriNKET-MGAH22 includes three polypeptides having the sequences of SEQ ID NO:192 (as in A49-F3′-TriNKET-MGAH22), SEQ ID NO:155 (as in A44-F3′-TriNKET-Trastuzumab), and SEQ ID NO:149 (as in A44-F3′-TriNKET-Trastuzumab).

Another exemplary TriNKET of the present disclosure is A44-F3′-KiH-TriNKET-MGAH22. A44-F3′-KiH-TriNKET-MGAH22 includes the same Her2-binding scFv (SEQ ID NO:171) as in A49-F3′-TriNKET-MGAH22 linked via a hinge comprising Ala-Ser to an Fc domain; and the same NKG2D-binding Fab fragment as in A44-F3′-TriNKET-Trastuzumab, the CH1 domain of which is connected to an Fc domain. The Fc domain linked to the scFv includes the “hole” substitutions of T366S, L368A, and Y407V, and the Fc domain linked to the Fab fragment includes the “knob” substitution of T366W. A44-F3′-KiH-TriNKET-MGAH22 includes three polypeptides having the sequences of SEQ ID NO:193 (as in A49-F3′-KiH-TriNKET-MGAH22), SEQ ID NO:148 (as in A44-F3′-KiH-TriNKET-Trastuzumab), and SEQ ID NO:149 (as in A44-F3′-TriNKET-Trastuzumab).

In certain embodiments, a TriNKET of the present disclosure is identical to one of the exemplary TriNKETs described above that includes the EW-RVT Fc mutations, except that the Fc domain linked to the NKG2D-binding Fab fragment comprises the substitutions of Q347R, D399V, and F405T, and the Fc domain linked to the HER2-binding scFv comprises matching substitutions K360E and K409W for forming a heterodimer. In certain embodiments, a TriNKET of the present disclosure is identical to one of the exemplary TriNKETs described above that includes the KiH Fc mutations, except that the Fc domain linked to the NKG2D-binding Fab fragment comprises the “hole” substitutions of T366S, L368A, and Y407V, and the Fc domain linked to the HER2-binding scFv comprises the “knob” substitution of T366W for forming a heterodimer.

In certain embodiments, a TriNKET of the present disclosure is identical to one of the exemplary TriNKETs described above, except that the Fc domain linked to the NKG2D-binding Fab fragment includes an S354C substitution in the CH3 domain, and the Fc domain linked to the HER2-binding scFv includes a matching Y349C substitution in the CH3 domain for forming a disulfide bond.

As described in International Application No. PCT/US2019/045561, the multi-specific binding proteins disclosed herein are effective in reducing tumor growth and killing cancer cells in in vitro assays and animal models. For example, A49-F3′-TriNKET-Trastuzumab is superior to trastuzumab in inducing NK cell-mediated cytotoxicity against various human cancer cell lines, such as 786-O cells that express low levels of HER2 (HER2+), H661 cells that express moderate levels of HER2 (HER2++), and SkBr3 cells that express high levels of HER2 (HER2+++). Furthermore, the multi-specific binding proteins do not significantly induce NK-mediated killing of healthy non-cancerous human cells (e.g., human cardiomyocytes).

### Production of Multi-Specific Binding Proteins

The multi-specific binding proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the immunoglobulin light chain can be cloned into a third expression vector; and the first, second, and third expression vectors can be stably transfected together into host cells to produce the multimeric proteins.

A skilled person in the art would appreciate that during production and/or storage of proteins, N-terminal glutamate (E) or glutamine (Q) can be cyclized to form a lactam (e.g., spontaneously or catalyzed by an enzyme present during production and/or storage). Accordingly, in some embodiments where the N-terminal residue of an amino acid sequence of a polypeptide is E or Q, a corresponding amino acid sequence with the E or Q replaced with pyroglutamate is also contemplated herein.

A skilled person in the art would also appreciate that during protein production and/or storage, the C-terminal lysine (K) of a protein can be removed (e.g., spontaneously or catalyzed by an enzyme present during production and/or storage). Such removal of K is often observed with proteins that comprise an Fc domain at their C-termini. Accordingly, in some embodiments where the C-terminal residue of an amino acid sequence of a polypeptide (e.g., an Fc domain sequence) is K, a corresponding amino acid sequence with the K removed is also contemplated herein.

To achieve the highest yield of the multi-specific binding protein, different ratios of the first, second, and third expression vector can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of the multi-specific binding protein. The multi-specific binding proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### Pharmaceutical Formulations

The present disclosure also provides pharmaceutical formulations that contain a therapeutically effective amount of a multi-specific binding protein disclosed herein (e.g., A49-F3′-TriNKET-Trastuzumab). The pharmaceutical formulation comprises one or more excipients and is maintained at a certain pH. The term “excipient,” as used herein, means any non-therapeutic agent added to the formulation to provide a desired physical or chemical property, for example, pH, osmolarity, viscosity, clarity, color, isotonicity, odor, sterility, stability, rate of dissolution or release, adsorption, or penetration.

### Excipients and pH

The one or more excipients in the pharmaceutical formulation of the present invention comprises a buffering agent. The term “buffering agent,” as used herein, refers to one or more components that when added to an aqueous solution is able to protect the solution against variations in pH when adding acid or alkali, or upon dilution with a solvent. In addition to phosphate buffers, glycinate, carbonate, citrate, histidine buffers and the like can be used, in which case, sodium, potassium or ammonium ions can serve as counterion.

In certain embodiments, the buffer or buffer system comprises at least one buffer that has a buffering range that overlaps fully or in part with the range of pH 5.5-7.4. In certain embodiments, the buffer has a pKa of about 6.0±0.5. In certain embodiments, the buffer comprises a histidine buffer. In certain embodiments, the histidine is present at a concentration of 5 to 100 mM, 10 to 100 mM, 15 to 100 mM, 20 to 100 mM, 5 to 50 mM, 10 to 50 mM, 15 to 100 mM, 20 to 100 mM, 5 to 25 mM, 10 to 25 mM, 15 to 25 mM, 20 to 25 mM, 5 to 20 mM, 10 to 20 mM, or 15 to 20 mM. In certain embodiments, the histidine is present at a concentration of 5 mM, 10 mM, 15 mM, 20 mM, 25 mM, or 50 mM. In certain embodiments, the histidine is present at a concentration of 20 mM.

The pharmaceutical formulation of the present invention may have a pH of 5.5 to 6.5. For example, in certain embodiments, the pharmaceutical formulation has a pH of 5.5 to 6.5 (i.e., 6.0±0.5), 5.6 to 6.4 (i.e., 6.0±0.4), 5.7 to 6.3 (i.e., 6.0±0.3), 5.8 to 6.2 (i.e., 6.0±0.2), 5.9 to 6.1 (i.e., 6.0±0.1), or 5.95 to 6.05 (i.e., 6.0±0.05). In certain embodiments, the pharmaceutical formulation has a pH of 5.5, 5.6, 5.7, 5.8, 5.9, 6.0, 6.1, 6.2, 6.3, 6.4, or 6.5. In certain embodiments, the pharmaceutical formulation has a pH of 6.0. Under the rules of scientific rounding, a pH greater than or equal to 5.95 and smaller than or equal to 6.05 is rounded as 6.0.

In certain embodiments, the buffer system of the pharmaceutical formulation comprises histidine at 10 to 25 mM, at a pH of 6.0±0.2. In certain embodiments, the buffer system of the pharmaceutical formulation comprises histidine at 20 mM, at a pH of 6.0±0.2. In certain embodiments, the buffer system of the pharmaceutical formulation comprises histidine at 10 to 25 mM, at a pH of 6.0±0.05. In certain embodiments, the buffer system of the pharmaceutical formulation comprises histidine at 20 mM, at a pH of 6.0±0.05.

The one or more excipients in the pharmaceutical formulation of the present invention further comprises a sugar or sugar alcohol. Sugars and sugar alcohols are useful in pharmaceutical formulations as a thermal stabilizer. In certain embodiments, the pharmaceutical formulation comprises a sugar, for example, a monosaccharide (glucose, xylose, or erythritol), a disaccharide (e.g., sucrose, trehalose, maltose, or galactose), or an oligosaccharide (e.g., stachyose). In specific embodiments, the pharmaceutical formulation comprises sucrose. In certain embodiments, the pharmaceutical composition comprises a sugar alcohol, for example, a sugar alcohol derived from a monosaccharide (e g , mannitol, sorbitol, or xylitol), a sugar alcohol derived from a disaccharide (e.g., lactitol or maltitol), or a sugar alcohol derived from an oligosaccharide. In specific embodiments, the pharmaceutical formulation comprises sorbitol.

The amount of the sugar or sugar alcohol contained within the formulation can vary depending on the specific circumstances and intended purposes for which the formulation is used. In certain embodiments, the pharmaceutical formulation comprises 50 to 300 mM, 50 to 250 mM, 100 to 300 mM, 100 to 250 mM, 150 to 300 mM, 150 to 250 mM, 200 to 300 mM, 200 to 250 mM, or 250 to 300 mM of the sugar or sugar alcohol. In certain embodiments, the pharmaceutical formulation comprises 50 mM, 75 mM, 100 mM, 125 mM, 150 mM, 200 mM, 250 mM, or 300 mM of the sugar or sugar alcohol. In specific embodiments, the pharmaceutical formulation comprises 250 mM of the sugar or sugar alcohol (e.g., sucrose or sorbitol).

The one or more excipients in the pharmaceutical formulation disclosed herein further comprises a surfactant. The term “surfactant,” as used herein, refers to a surface active molecule containing both a hydrophobic portion (e.g., alkyl chain) and a hydrophilic portion (e.g., carboxyl and carboxylate groups). Surfactants are useful in pharmaceutical formulations for reducing aggregation of a therapeutic protein. Surfactants suitable for use in the pharmaceutical formulations are generally non-ionic surfactants and include, but are not limited to, polysorbates (e.g. polysorbates 20 or 80); poloxamers (e.g. poloxamer 188); sorbitan esters and derivatives; Triton; sodium laurel sulfate; sodium octyl glycoside; lauryl-, myristyl-, linoleyl-, or stearyl-sulfobetadine; lauryl-, myristyl-, linoleyl- or stearyl-sarcosine; linoleyl-, myristyl-, or cetyl-betaine; lauramidopropyl-cocamidopropyl-, linoleamidopropyl-, myristamidopropyl-, palmidopropyl-, or isostearamidopropylbetaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethylene glycol, polypropyl glycol, and copolymers of ethylene and propylene glycol (e.g., Pluronics, PF68 etc.). In certain embodiments, the surfactant is a polysorbate. In certain embodiments, the surfactant is polysorbate 80.

The amount of a non-ionic surfactant contained within the pharmaceutical formulation of the present invention may vary depending on the specific properties desired of the formulation, as well as the particular circumstances and purposes for which the formulations are intended to be used. In certain embodiments, the pharmaceutical formulation comprises 0.005% to 0.5%, 0.005% to 0.2%, 0.005% to 0.1%, 0.005% to 0.05%, 0.005% to 0.02%, 0.005% to 0.01%, 0.01% to 0.5%, 0.01% to 0.2%, 0.01% to 0.1%, 0.01% to 0.05%, or 0.01% to 0.02% of the non-ionic surfactant (e.g., polysorbate 80). In certain embodiments, the pharmaceutical formulation comprises 0.005%, 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, or 0.5% of the non-ionic surfactant (e.g., polysorbate 80).

In certain embodiments, the pharmaceutical formulation is isotonic. An “isotonic” formulation is one which has essentially the same osmotic pressure as human blood. Isotonic formulations generally have an osmotic pressure from about 250 to 350 mOsmol/kgH₂O. Isotonicity can be measured using a vapor pressure or ice-freezing type osmometer. In certain embodiments, the osmolarity of the pharmaceutical formulation is 250 to 350 mOsmol/kgH₂O. In certain embodiments, the osmolarity of the pharmaceutical formulation is 300 to 350 mOsmol/kgH₂O.

Substances such as sugar, sugar alcohol, and NaCl can be included in the pharmaceutical formulation for desired osmolarity. In certain embodiments, the concentration of NaCl in the pharmaceutical formulation, if any, is equal to or lower than 10 mM, 9 mM, 8 mM, 7 mM, 6 mM, 5 mM, 4 mM, 3 mM, 2 mM, 1 mM, 0.5 mM, 0.1 mM, 50 μM, 10 μM, 5 μM, or 1 μM. In certain embodiments, the concentration of NaCl in the pharmaceutical formulation is below the detection limit. In certain embodiments, no NaCl salt is added when preparing the pharmaceutical formulation.

The pharmaceutical formulation of the present invention may further comprise one or more other substances, such as a bulking agent or a preservative. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents. A preservative reduces bacterial action and may, for example, facilitate the production of a multi-use (multiple-dose) formulation.

### Exemplary Formulations

In certain embodiments, the pharmaceutical formulation of the present invention comprises the multi-specific binding protein, histidine, a sugar or sugar alcohol (e.g., sucrose or sorbitol), and a polysorbate (e.g., polysorbate 80), at pH 5.5 to 6.5.

In certain embodiments, the pharmaceutical formulation comprises 10 to 50 mg/mL of the multi-specific binding protein, 10 to 25 mM of histidine, 200 to 300 mM of a sugar or sugar alcohol (e.g., sucrose or sorbitol), and 0.005% to 0.05% of a polysorbate (e.g., polysorbate 80), at pH 5.5 to 6.5. In certain embodiments, the pharmaceutical formulation comprises 10 to 50 mg/mL of the multi-specific binding protein, 20 mM of histidine, 250 mM of a sugar or sugar alcohol (e.g., sucrose or sorbitol), and 0.01% of a polysorbate (e.g., polysorbate 80), at pH 5.5 to 6.5. In certain embodiments, the pharmaceutical formulation comprises 10 to 50 mg/mL of the multi-specific binding protein, 20 mM of histidine, 250 mM of a sugar or sugar alcohol (e.g., sucrose or sorbitol), and 0.01% of a polysorbate (e.g., polysorbate 80), at pH 5.8 to 6.2. In certain embodiments, the pharmaceutical formulation comprises 10 to 50 mg/mL of the multi-specific binding protein, 20 mM of histidine, 250 mM of a sugar or sugar alcohol (e.g., sucrose or sorbitol), and 0.01% of a polysorbate (e.g., polysorbate 80), at pH 5.95 to 6.05.

In certain embodiments, the pharmaceutical formulation comprises 10 to 50 mg/mL of the multi-specific binding protein, 10 to 25 mM of histidine, 200 to 300 mM of sucrose, and 0.005% to 0.05% of polysorbate 80, at pH 5.5 to 6.5. In certain embodiments, the pharmaceutical formulation comprises 10 to 50 mg/mL of the multi-specific binding protein, 20 mM of histidine, 250 mM of sucrose, and 0.01% of polysorbate 80, at pH 5.5 to 6.5. In certain embodiments, the pharmaceutical formulation comprises 10 to 50 mg/mL of the multi-specific binding protein, 20 mM of histidine, 250 mM of sucrose, and 0.01% of polysorbate 80, at pH 5.8 to 6.2. In certain embodiments, the pharmaceutical formulation comprises 10 to 50 mg/mL of the multi-specific binding protein, 20 mM of histidine, 250 mM of sucrose, and 0.01% of polysorbate 80, at pH 5.95 to 6.05.

In certain embodiments, the pharmaceutical formulation comprises 10 to 50 mg/mL of the multi-specific binding protein, 10 to 25 mM of histidine, 200 to 300 mM of sorbitol, and 0.005% to 0.05% of polysorbate 80, at pH 5.5 to 6.5. In certain embodiments, the pharmaceutical formulation comprises 10 to 50 mg/mL of the multi-specific binding protein, 20 mM of histidine, 250 mM of sorbitol, and 0.01% of polysorbate 80, at pH 5.5 to 6.5. In certain embodiments, the pharmaceutical formulation comprises 10 to 50 mg/mL of the multi-specific binding protein, 20 mM of histidine, 250 mM of sorbitol, and 0.01% of polysorbate 80, at pH 5.8 to 6.2. In certain embodiments, the pharmaceutical formulation comprises 10 to 50 mg/mL of the multi-specific binding protein, 20 mM of histidine, 250 mM of sorbitol, and 0.01% of polysorbate 80, at pH 5.95 to 6.05.

### Stability of the Multi-Specific Binding Protein

The pharmaceutical formulations of the present invention exhibit high levels of stability. A pharmaceutical formulation is stable when the multi-specific binding protein within the formulation retains an acceptable degree of physical property, chemical structure, and/or biological function after storage under defined conditions.

Stability can be measured by determining the percentage of the multi-specific binding protein in the formulation that remains in a native conformation after storage for a defined amount of time at a defined temperature. The percentage of a protein in a native conformation can be determined by, for example, size exclusion chromatography (e.g., size exclusion high performance liquid chromatography), where a protein in the native conformation is not aggregated (eluted in a high molecular weight fraction) or degraded (eluted in a low molecular weight fraction). In certain embodiments, more than 95%, 96%, 97%, 98%, or 99% of the multi-specific binding protein has native conformation, as determined by size-exclusion chromatography, after incubation at 4° C. for 3 weeks. In certain embodiments, more than 90%, 91%, 92%, 93%, 94%, 95%, 96%, 97%, 98%, or 99% of the multi-specific binding protein has native conformation, as determined by size-exclusion chromatography, after incubation at 50° C. for 3 weeks. In certain embodiments, less than 0.5%, 0.6%, 0.7%, 0.8%, 0.9%, or 1% of the multi-specific binding protein forms a high molecular weight complex (i.e., having a higher molecular weight than the native protein), as determined by size-exclusion chromatography, after incubation at 4° C. for 3 weeks. In certain embodiments, less than 1%, 2%, 3%, 4%, or 5% of the multi-specific binding protein form a high molecular weight complex (i.e., having a higher molecular weight than the native protein), as determined by size-exclusion chromatography, after incubation at 50° C. for 3 weeks. In certain embodiments, less than 0.5%, 0.6%, 0.7%, 0.8%, 0.9%, or 1% of the multi-specific binding protein is degraded (i.e., having a lower molecular weight than the native protein), as determined by size-exclusion chromatography, after incubation at 4° C. for 3 weeks. In certain embodiments, less than 1%, 1.5%, 2%, 2.5%, or 3% of the multi-specific binding protein is degraded (i.e., having a lower molecular weight than the native protein), as determined by size-exclusion chromatography, after incubation at 50° C. for 3 weeks.

Stability can also be measured by determining the percentage of multi-specific binding protein present in a more acidic fraction (“acidic form”) relative to the main fraction of protein (“main charge form”). While not wishing to be bound by theory, deamidation of a protein may cause it to become more negatively charged and thus more acidic relative to the non-deamidated protein (see, e.g., Robinson, Protein Deamidation, (2002) PNAS 99(8):5283-88). The percentage of the acidic form of a protein can be determined by ion exchange chromatography (e.g., cation exchange high performance liquid chromatography) or imaged capillary isoelectric focusing (icIEF). In certain embodiments, at least 50%, 60%, 70%, 80%, or 90% of the multi-specific binding protein in the pharmaceutical formulation is in the main charge form after incubation at 4° C. for 3 weeks. In certain embodiments, at least 15%, 20%, 25%, 30%, 40%, 50%, 60%, 70%, 80%, or 90% of the multi-specific binding protein in the pharmaceutical formulation is in the main charge form after incubation at 50° C. for 3 weeks. In certain embodiments, no more than 10%, 20%, 30%, 40%, or 50% of the multi-specific binding protein in the pharmaceutical formulation is in an acidic form after incubation at 4° C. for 3 weeks. In certain embodiments, no more than 10%, 20%, 30%, 40%, 50%, 60%, 70%, 75%, 80%, or 85% of the multi-specific binding protein in the pharmaceutical formulation is in an acidic form after incubation at 50° C. for 3 weeks.

Stability can also be measured by determining the purity of the multi-specific binding protein by electrophoresis after denaturing the protein with sodium dodecyl sulfate (SDS). The protein sample can be denatured in the presence or absence of an agent that reduces protein disulfide bonds (e.g., β-mercaptoethanol). In certain embodiments, the purity of the multi-specific binding protein in the pharmaceutical formulation, as measured by capillary electrophoresis after denaturing the protein sample under reducing conditions (e.g., in the presence of β-mercaptoethanol), is at least 95%, 96%, 97%, 98%, or 99% after incubation at 4° C. for 3 weeks. In certain embodiments, the purity of the multi-specific binding protein in the pharmaceutical formulation, as measured by capillary electrophoresis after denaturing the protein sample under reducing conditions (e.g., in the presence of β-mercaptoethanol), is at least 90%, 91%, 92%, 93%, 94%, 95%, 96%, 97%, 98%, or 99% after incubation at 50° C. for 3 weeks. In certain embodiments, the purity of the multi-specific binding protein in the pharmaceutical formulation, as measured by capillary electrophoresis after denaturing the protein sample under non-reducing conditions, is at least 95%, 96%, 97%, 98%, or 99% after incubation at 4° C. for 3 weeks. In certain embodiments, the purity of the multi-specific binding protein in the pharmaceutical formulation, as measured by capillary electrophoresis after denaturing the protein sample under non-reducing conditions, is at least 85%, 86%, 87%, 88%, 89%, 90%, 91%, 92%, 93%, 94%, 95%, 96%, 97%, 98%, or 99% after incubation at 50° C. for 3 weeks.

Stability can also be measured by determining the parameters of a protein solution by dynamic light scattering. The Z-average and polydispersity index (PDI) values indicate the average diameter of particles in a solution and these measures increase when aggregates are present in the solution. The monomer % Pd value indicates the spread of different monomers detected, where lower values indicate a monodispere solution, which is preferred. The monomer size detected by DLS is useful in confirming that the main population is monomer and to characterize any higher order aggregates that may be present. In certain embodiments, the Z-average value of the pharmaceutical formulation does not increase by more than 5%, 10%, or 15% after incubation at 4° C. for 3 weeks. In certain embodiments, the Z-average value of the pharmaceutical formulation does not increase by more than 5%, 10%, 15%, 20%, or 25% after incubation at 50° C. for 3 weeks. In certain embodiments, the PDI value of the pharmaceutical formulation does not increase by more than 10%, 20%, 30%, 40%, or 50% after incubation at 4° C. for 3 weeks. In certain embodiments, the PDI value of the pharmaceutical formulation does not increase by more than 2-fold, 3-fold, 4-fold, or 5-fold after incubation at 50° C. for 3 weeks.

Exemplary methods to determine stability of the multi-specific binding protein in the pharmaceutical formulation are described in Example 1 of the present disclosure. Additionally, stability of the protein can be assessed by measuring the binding affinity of the multi-specific binding protein to its targets or the biological activity of the multi-specific binding protein in certain in vitro assays, such as the NK cell activation assays and cytotoxicity assays described in WO 2018/152518.

### Dosage Forms

The pharmaceutical formulation can be prepared and stored as a liquid formulation or a lyophilized form. In certain embodiments, the pharmaceutical formulation is a liquid formulation for storage at 2-8° C. (e.g., 4° C.) or a frozen formulation for storage at −20° C. or lower. The sugar or sugar alcohol in the formulation is used as a lyoprotectant.

Prior to pharmaceutical use, the pharmaceutical formulation can be diluted or reconstituted in an aqueous carrier is suitable for the route of administration. Other exemplary carriers include sterile water for injection (SWFI), bacteriostatic water for injection (BWFI), a pH buffered solution (e.g., phosphate-buffered saline), sterile saline solution, Ringer's solution, or dextrose solution. For example, when the pharmaceutical formulation is prepared for intravenous administration, the pharmaceutical formulation can be diluted in a 0.9% sodium chloride (NaCl) solution. In certain embodiments, the diluted pharmaceutical formulation is isotonic and suitable for administration by intravenous infusion.

The pharmaceutical formulation comprises the multi-specific binding protein at a concentration suitable for storage. In certain embodiments, the pharmaceutical formulation comprises the multi-specific binding protein at a concentration of 10-50 mg/mL, 10-40 mg/mL, 10-30 mg/mL, 10-25 mg/mL, 10-20 mg/mL, 10-15 mg/mL, 15-50 mg/mL, 15-40 mg/mL, 15-30 mg/mL, 15-25 mg/mL, 15-20 mg/mL, 20-50 mg/mL, 20-40 mg/mL, 20-30 mg/mL, 20-25 mg/mL, 30-50 mg/mL, 30-40 mg/mL, or 40-50 mg/mL. In certain embodiments, the pharmaceutical formulation comprises the multi-specific binding protein at a concentration of 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 25 mg/mL, 30 mg/mL, 35 mg/mL, 40 mg/mL, 45 mg/mL, or 50 mg/mL.

In certain embodiments, the pharmaceutical formulation is packaged in a container (e.g., a vial, bag, pen, or syringe). In certain embodiments, the formulation may be a lyophilized formulation or a liquid formulation. In certain embodiments, the amount of multi-specific binding protein in the container is suitable for administration as a single dose. In certain embodiments, the amount of multi-specific binding protein in the container is suitable for administration in multiple doses. In certain embodiments, the pharmaceutical formulation comprises the multi-specific binding protein at an amount of 0.1 to 2000 mg. In certain embodiments, the pharmaceutical formulation comprises the multi-specific binding protein at an amount of 1 to 2000 mg, 10 to 2000 mg, 20 to 2000 mg, 50 to 2000 mg, 100 to 2000 mg, 200 to 2000 mg, 500 to 2000 mg, 1000 to 2000 mg, 0.1 to 1000 mg, 1 to 1000 mg, 10 to 1000 mg, 20 to 1000 mg, 50 to 1000 mg, 100 to 1000 mg, 200 to 1000 mg, 500 to 1000 mg, 0.1 to 500 mg, 1 to 500 mg, 10 to 500 mg, 20 to 500 mg, 50 to 500 mg, 100 to 500 mg, 200 to 500 mg, 0.1 to 200 mg, 1 to 200 mg, 10 to 200 mg, 20 to 200 mg, 50 to 200 mg, 100 to 200 mg, 0.1 to 100 mg, 1 to 100 mg, 10 to 100 mg, 20 to 100 mg, 50 to 100 mg, 0.1 to 50 mg, 1 to 50 mg, 10 to 50 mg, 20 to 50 mg, 0.1 to 20 mg, 1 to 20 mg, 10 to 20 mg, 0.1 to 10 mg, 1 to 10 mg, or 0.1 to 1 mg. In certain embodiments, the pharmaceutical formulation comprises the multi-specific binding protein at an amount of 0.1 mg, 1 mg, 2 mg, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 400 mg, 500 mg, 600 mg, 700 mg, 800 mg, 900 mg, 1000 mg, 1500 mg, or 2000 mg.

### Dosage Regimens and Therapeutic Uses

In another aspect, the present disclosure provides a method for treating cancer, the method comprising administering to a subject in need thereof a multi-specific binding protein disclosed herein (e.g., A49-F3′-TriNKET-Trastuzumab) in an initial four-week treatment cycle on Day 1, Day 8, and Day 15. In certain embodiments, the multi-specific binding protein is administered to the subject only on these three days in the initial four-week treatment cycle. In specific embodiments, the multi-specific binding protein is not administered to the subject on Day 22. This regimen is a dose intensification schedule, which is designed to reach maximal saturation of the target as early as possible during the course of the treatment while minimizing the infusion burden for the patient.

In certain embodiments, the method further comprises administering to the subject, after the initial treatment cycle, the multi-specific binding protein in one or more subsequent four-week treatment cycles, wherein the multi-specific binding protein is administered on Day 1 and Day 15 in each subsequent treatment cycle. In certain embodiments, the multi-specific binding protein is administered to the subject only on these two days in each subsequent four-week treatment cycle. In specific embodiments, the multi-specific binding protein is not administered to the subject on Day 8 or Day 22. The subsequent treatment cycles, in which the subject receives administration of the multi-specific binding protein once every two weeks, are designed to maintain a certain level of the multi-specific binding protein in the subject. In certain embodiments, the subject receives at least 1, 2, 3, 4, 5, 6, 7, 8, 9, 10, 11, 12, 13, 14, or 15 subsequent treatment cycles. In certain embodiments, the subject receives subsequent treatment cycles until regression of the cancer.

In certain embodiments, one or more doses in the initial and subsequent treatment cycles comprise the multi-specific binding protein at an amount of 0.1-20 mg/kg, 0.1-10 mg/kg, 0.1-5 mg/kg, 0.1-2 mg/kg, 0.1-1 mg/kg, 0.1-0.5 mg/kg, 0.1-0.2 mg/kg, 0.2-20 mg/kg, 0.2-10 mg/kg, 0.2-5 mg/kg, 0.2-2 mg/kg, 0.2-1 mg/kg, 0.2-0.5 mg/kg, 0.5-20 mg/kg, 0.5-10 mg/kg, 0.5-5 mg/kg, 0.5-2 mg/kg, 0.5-1 mg/kg, 1-20 mg/kg, 1-10 mg/kg, 1-5 mg/kg, or 1-2 mg/kg. In certain embodiments, one or more doses in the initial and subsequent treatment cycles comprise the multi-specific binding protein at an amount selected from the group consisting of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.5 mg/kg, 2 mg/kg, 2.5 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, and 20 mg/kg.

In certain embodiments, each of the doses in the initial and subsequent treatment cycles comprises the multi-specific binding protein at an amount selected from the group consisting of 0.1-20 mg/kg, 0.1-10 mg/kg, 0.1-5 mg/kg, 0.1-2 mg/kg, 0.1-1 mg/kg, 0.1-0.5 mg/kg, 0.1-0.2 mg/kg, 0.2-20 mg/kg, 0.2-10 mg/kg, 0.2-5 mg/kg, 0.2-2 mg/kg, 0.2-1 mg/kg, 0.2-0.5 mg/kg, 0.5-20 mg/kg, 0.5-10 mg/kg, 0.5-5 mg/kg, 0.5-2 mg/kg, 0.5-1 mg/kg, 1-20 mg/kg, 1-10 mg/kg, 1-5 mg/kg, and 1-2 mg/kg. In certain embodiments, each of the doses in the initial and subsequent treatment cycles comprises the multi-specific binding protein at a same amount selected from the group consisting of 0.1-20 mg/kg, 0.1-10 mg/kg, 0.1-5 mg/kg, 0.1-2 mg/kg, 0.1-1 mg/kg, 0.1-0.5 mg/kg, 0.1-0.2 mg/kg, 0.2-20 mg/kg, 0.2-10 mg/kg, 0.2-5 mg/kg, 0.2-2 mg/kg, 0.2-1 mg/kg, 0.2-0.5 mg/kg, 0.5-20 mg/kg, 0.5-10 mg/kg, 0.5-5 mg/kg, 0.5-2 mg/kg, 0.5-1 mg/kg, 1-20 mg/kg, 1-10 mg/kg, 1-5 mg/kg, and 1-2 mg/kg.

In certain embodiments, each of the doses in the initial and subsequent treatment cycles comprises the multi-specific binding protein at an amount selected from the group consisting of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.5 mg/kg, 2 mg/kg, 2.5 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, and 20 mg/kg. In certain embodiments, each of the doses in the initial and subsequent treatment cycles comprises the multi-specific binding protein at a same amount selected from the group consisting of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.5 mg/kg, 2 mg/kg, 2.5 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, and 20 mg/kg.

In certain embodiments, each of the doses in the initial and subsequent treatment cycles comprises the multi-specific binding protein at an amount selected from the group consisting of 5.2×10⁻⁵ mg/kg, 1.6×10⁻⁴ mg/kg, 5.2×10⁻⁴ mg/kg, 1.6×10⁻³ mg/kg, 5.2×10⁻³ mg/kg, 1.6×10⁻² mg/kg, 5.2×10⁻² mg/kg, 1.6×10⁻¹ mg/kg, 0.52 mg/kg, 1.6 mg/kg, 5.2 mg/kg, 10 mg/kg, and 20 mg/kg. In certain embodiments, each of the doses in the initial and subsequent treatment cycles comprises the multi-specific binding protein at a same amount selected from the group consisting of 5.2×10⁻⁵ mg/kg, 1.6×10⁻⁴ mg/kg, 5.2×10⁻⁴ mg/kg, 1.6×10⁻³ mg/kg, 5.2×10⁻³ mg/kg, 1.6×10⁻² mg/kg, 5.2×10⁻² mg/kg, 1.6×10⁻¹ mg/kg, 0.52 mg/kg, 1 mg/kg, 1.6 mg/kg, 5.2 mg/kg, 10 mg/kg, 20 mg/kg, and 50 mg/kg.

In certain embodiments, the multi-specific binding protein is administered intravenously. For example, in certain embodiments, the multi-specific binding protein is administered by intravenous infusion, e.g., with a prefilled bag, a prefilled pen, or a prefilled syringe. In certain embodiments, the multi-speicific binding protein, in a pharmaceutical formulation disclosed herein, is diluted prior to administration. For example, in certain embodiments, the pharmaceutical formulation is diluted with sodium chloride and is administered intravenously from a 250 ml saline bag. The intravenous infusion may be for about one hour (e.g., 50 to 80 minutes). In certain embodiments, the bag is connected to a channel comprising a tube and/or a needle.

The types of cancer that can be treated with the multi-specific binding protein or pharmaceutical formulation disclosed herein include but are not limited to breast cancer, thyroid cancer, gastric cancer, renal cell carcinoma, adenocarcinoma of the lung, prostate cancer, cholangiocarcinoma, uterine cancer, pancreatic cancer, colorectal cancer, ovarian cancer, cervical cancer, head and neck cancer, NSCLC, glioblastoma, esophageal cancer, squamous carcinoma of the skin, carcinoma of the salivary gland, biliary tract cancer, lung squamous, mesothelioma, liver cancer, sarcoma, bladder cancer, and gallbladder cancer. In certain embodiments, the cancer is a solid tumor. In certain embodiments, the cancer is a locally advanced or metastatic solid tumor. In certain embodiments, the cancer is urothelial bladder cancer or metastatic breast cancer,.

In certain embodiments, the subject treated by the method disclosed herein has HER2-positive cancer. Methods of determining HER2 expression in a cancer include but are not limited to immunohistochemistry. Anti-HER2 antibodies (e.g., Ventana 4B5 antibody and Bond Oracle CB11 antibody) have been approved by the FDA for detecting HER2, and immunohistochemistry kits (e.g., HercepTest™) are commercially available. The level of HER2 expression in a tumor sample, as detected by immunohistochemistry, can be quantified and scored as 1+, 2+, or 3+ according to the ASCO/CAP guideline (Wolff et al., (2007) J. Clin. Oncol. 25(1):118-45). In certain embodiments, the subject treated by the method disclosed herein has a tumor with HER2 level scored as 1+, 2+, or 3+. In certain embodiments, the subject treated by the method disclosed herein has a tumor with HER2 level scored as 2+or 3+. In certain embodiments, the subject treated by the method disclosed herein has a tumor with HER2 level scored as 3+. In certain embodiments, the HER2 level is determined by immunohistochemistry (e.g., HercepTest™). In certain embodiments, the subject treated by the method disclosed herein has a tumor that shows HER2 expression at least as a faint/barely perceptible membrane staining detected in at least or more than 10% of the tumor cells. In certain embodiments, the subject treated by the method disclosed herein has a tumor that shows HER2 expression at least as a weak to moderate complete membrane staining detected in at least or more than 10% of the tumor cells. In certain embodiments, the subject treated by the method disclosed herein has a tumor that shows HER2 expression at least as a strong complete membrane staining detected in at least or more than 10% of the tumor cells.

In certain embodiments, the subject treated by the method disclosed herein has cancer harboring ERBB2 gene amplification. ERBB2 gene amplification is generally correlated with HER2 overexpression and determining whether ERBB2 gene is amplified in a cancer tissue sample may help reduce false-positive results from immunohistochemistry of the same sample (see, e.g., Sarode et al., (2015) Arch. Pathol. Lab. Med. 139:922-28). Methods of detecting gene amplification include but are not limited to fluorescent in situ hybridization (FISH), chromogenic in situ hybridization (CISH), quantitative PCR, and DNA sequencing. In certain embodiments, ERBB2 gene amplification is determined by FISH. In certain embodiments, ERBB2 gene amplification is determined by DNA sequencing (e.g., deep sequencing).

In certain embodiments, the subject treated in accordance with the methods disclosed herein has not received prior therapy for treating the cancer. In certain embodiment, the subject treated in accordance with the methods disclosed herein has not received prior chemotherapy or immunotherapy for treating the cancer. In certain embodiments, the subject treated in accordance with the methods disclosed herein has received a prior therapy (e.g., a chemotherapy or immunotherapy) but continues to experience cancer progression despite the prior therapy. In certain embodiments, the subject treated in accordance with the methods disclosed herein has experienced cancer regression after receiving a prior therapy (e.g., a chemotherapy or immunotherapy), but later experienced cancer relapse. In certain embodiments, the subject treated in accordance with the methods disclosed herein is intolerant to a prior therapy (e.g., a chemotherapy or immunotherapy).

In certain embodiments, the subject treated in accordance with the methods disclosed herein meets all the inclusion criteria of a clinical trial cohort (e.g., the accelerated titration cohort, the “3+3” dose escalation cohort, the safety/PK/PD expansion cohorts, the urothelial bladder cancer (UBC) cohort, the metastatic breast cancer (MBC) cohort, the Basket solid tumors with high HER2 expression (HER2 3+) cohort, or the Combination therapy with pembrolizumab cohort) described in Example 3. In certain embodiments, the subject treated in accordance with the methods disclosed herein does not meet any the exclusion criteria described in Example 3.

The multi-specific binding protein disclosed herein can be used as a monotherapy or in combination with one or more therapies. In certain embodiments, the multi-specific binding protein is used as a monotherapy in accordance with the dosage regimen disclosed herein. In other embodiments, the multi-specific binding protein is used in combination with one or more therapies, wherein the multi-specific binding protein is administered in accordance with the dosage regimen disclosed herein and the one or more therapies are administered in accordance with a dosage regimen known to be suitable for treating the particular subject with the particular cancer. In certain embodiments, the method of treatment disclosed herein is used as an adjunct to surgical removal of the primary lesion.

Exemplary therapeutic agents that may be used in combination with the multi-specific binding protein include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma (IFN-γ), colony stimulating factor-1, colony stimulating factor-2, denileukin diftitox, interleukin-2, luteinizing hormone releasing factor and variations of the aforementioned agents that may exhibit differential binding to their cognate receptors, or increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T lymphocyte-associated antigen 4 (CTLA4), (ii) programmed cell death protein 1 (PD1), (iii) PDL1, (iv) LAGS, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

In certain embodiments, the method of the present invention further comprises administering to the subject an anti-PD-1 antibody. Many anti-PD-1 antibodies have been developed for therapeutic purposes and are described in, for example, Gong et al., (2018) J. ImmunoTher Cancer (2018) 6:8. In certain embodiments, the anti-PD-1 antibody is pembrolizumab. In certain embodiments, 200 mg of pembrolizumab is administered on Day 1 of the initial treatment cycle. In certain embodiments, if the subject receives one or more subsequent treatment cycles, 200 mg of pembrolizumab is administered once every three weeks in the subsequent treatment cycles, starting from Day 1 of the first subsequent treatment cycle.

In certain embodiments, the method of treatment disclosed herein results in a disease response or improved survival of the subject or patient. For example, in certain embodiments, the disease response is a complete response, a partial response, or a stable disease. In certain embodiments, the improved survival is improved progression-free survival (PFS) or overall survival. Improvement (e.g., in PFS) can be determined relative to a period prior to initiation of the treatment of the present disclosure. Methods of determining disease response (e.g., complete response, partial response, or stable disease) and patient survival (e.g., PFS, overall survival) for BTC (e.g., advanced BTC, metastatic BTC), or biliary tract tumor therapy, are routine in the art and are contemplated herein. In some embodiments, disease response is evaluated according to RECIST 1.1 after subjecting the treated patient to contrast-enhanced computed tomography (CT) or magnetic resonance imaging (MRI) of the affected area (e.g., chest/abdomen and pelvis covering the area from the superior extent of the thoracic inlet to the symphysis pubis).

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1

### Formulation, Packaging, and Storage of A49-F3′-TriNKET-Trastuzumab

The formulations listed in Table 12 were evaluated, in duplicate and randomized, to assess the effects of the pH and excipients on the stability of A49-F3′-TriNKET-Trastuzumab (Kermit BDS lot 7443-C3, 11.9 mg/mL). A49-F3′-TriNKET-Trastuzumab underwent buffer exchange into the respective buffer and excipient combinations by centrifugal ultrafiltration (Amicon Ultra-4 30 k devices MWCO) to a target concentration of 30 mg/mL. Following the final buffer exchange and confirmation of the target concentration, each formulated sample was filter sterilized using a 0.22 μm EMD Millipore Ultrafree—CL centrifugal filter devices with Durapore membrane (Fisher Scientific Cat. #UFC40GVOS). Following sterile filtration, each formulation was handled aseptically in a laminar flow hood. The formulated samples were spiked with polysorbate 80 (PS80) to a final concentration of 0.01%. An aliquot of each formulation was removed for time zero testing, and the remaining material was split into two equal sized aliquots into depyrogenated Type 1 borosilicate glass vials, 2 mL×13 mm (West Pharmaceuticals Cat. #68000377), stoppered with 13 mm Fluorotec stoppers (West Pharmaceuticals Cat. #19700302), and sealed. The time zero aliquots were used for the initial time point testing per Table 13. One vial was stored at 2-8° C. and the other vial was placed at 50° C. for a 3-week accelerated stability study. Following the 3-week incubation, the 2-8° C. and 50° C. samples were analyzed according to the test methods indicated in Table 13.

TABLE 12
Formulations evaluated

| Buffer | Excipient | Surfactant | pH | | | | | Conditions | Conc. |
|---|---|---|---|---|---|---|---|---|---|
| 20 mM | 250 mM | 0.01% | 5.5 | 5.8 | 6.0 | 6.2 | 6.5 | Time Zero, | 30 mg/mL |
| Histidine | Sorbitol | PS80 | | | | | | 3-week | |
| | 250 mM | | | | | | | incubation | |
| | Sucrose | | | | | | | at 2-8° C., | |
| | 75 mM NaCl, | | | | | | | 3-week | |
| | 125 mM | | | | | | | incubation | |
| | Sorbitol | | | | | | | at 50° C. | |
| | 75 mM NaCl, | | | | | | | | |
| | 125 mM | | | | | | | | |
| | Sucrose | | | | | | | | |

TABLE 13
Assay panel used in formulation evaluation

| | | 3-week incubation | |
|---|---|---|---|
| Test Method | Initial | 2-8° C. | 50° C. |
| Appearance | X | X | X |
| Concentration, | X | X | X |
| ( [symbol] = 1.595 | | | |
| mL/cm*mg) | | | |
| pH | X | X | X |
| DLS | X | X | X |
| SEC-HPLC | X | X | X |
| Osmo | X | X | X |
| CE-SDS | | X | X |
| icIEF | | X | X |

An accelerated stability study of A49-F3′-TriNKET-Trastuzumab was executed, in which A49-F3′-TriNKET-Trastuzumab was prepared in 20 formulations as shown in Table 12. Samples were run in duplicate and incubated for 3 weeks at 2-8° C. and 50° C. At time zero and upon conclusion of the 3-week incubation, testing of each formulated sample was performed using the assays as outlined in Table 13. All formulations behaved similarly and were within expectation as evaluated by appearance, concentration, pH, and osmolality.

### Appearance

Samples were viewed in ambient laboratory conditions against a black and white background before the sample vials were opened. All samples were absent of visible particulates at both time zero and three week conditions.

### Ultraviolet Concentration Determination

Protein concentration by ultraviolet (UV) absorption at optical density (OD) 280 nm was determined for each sample and condition. Protein concentrations at time zero, after 3-week incubation at 2-8° C., and after 3-week incubation at 50° C. are summarized in Table 14.

### pH Determination

The pH was determined for each sample and condition. The pH values at time zero, after 3-week incubation at 2-8° C., and after 3-week incubation at 50° C. are summarized in Table 15.

### Dynamic Light Scattering

Dynamic Light Scattering (DLS) samples were collected at 25° C., following a 300 second equilibration. Five measurements were collected for each sample. Z-average values at time zero, after 3-week incubation at 2-8° C., and after 3-week incubation at 50° C. are summarized in Table 16.

Average polydispersity index (% PDI) was also recorded. The % PDI values at time zero, after 3-week incubation at 2-8° C., and after 3-week incubation at 50° C. are summarized in Table 17.

Further DLS analysis of A49-F3′-TriNKET-Trastuzumab in the samples was performed. The average percentage of monomer polydispersity (% PD) at time zero, after 3-week incubation at 2-8° C., and after 3-week incubation at 50° C. are summarized in Table 18. The average monomer size values at time zero, after 3-week incubation at 2-8° C., and after 3-week incubation at 50° C. are summarized in Table 19.

TABLE 14
Calculated protein concentration from UV absorption

| | Excipient/ | | Time Zero | | 3-week incubation | | | |
|---|---|---|---|---|---|---|---|---|
| Buffer | Surfactant | pH | 1 | 2 | 2-8° C. | | 50° C. | |
| 20 mM | 250 mM | 5.5 | 32.2 | 32.5 | 33.0 | 32.8 | 32.4 | 32.7 |
| Histidine | Sorbitol, | 5.8 | 30.2 | 32.4 | 31.3 | 32.6 | 31.3 | 32.5 |
| | 0.01% PS80 | 6.0 | 31.0 | 29.9 | 31.0 | 31.1 | 30.9 | 31.7 |
| | | 6.2 | 28.0 | 32.1 | 29.4 | 31.9 | 28.6 | 31.2 |
| | | 6.5 | 31.0 | 27.7 | 33.1 | 28.9 | 33.3 | 28.2 |
| 20 mM | 250 mM | 5.5 | 31.0 | 30.9 | 31.8 | 33.2 | 31.4 | 31.6 |
| Histidine | Sucrose, | 5.8 | 29.1 | 30.2 | 30.6 | 30.5 | 31.0 | 29.8 |
| | 0.01% PS80 | 6.0 | 31.0 | 29.7 | 33.1 | 30.3 | 32.2 | 30.4 |
| | | 6.2 | 30.6 | 31.2 | 31.5 | 32.2 | 31.4 | 32.5 |
| | | 6.5 | 31.4 | 30.6 | 31.6 | 32.4 | 31.0 | 31.6 |
| 20 mM | 75 mM NaCl, | 5.5 | 28.7 | 31.3 | 30.1 | 31.1 | 29.6 | 30.7 |
| Histidine | 125 mM | 5.8 | 31.9 | 29.5 | 32.1 | 30.7 | 32.5 | 31.4 |
| | Sorbitol, | 6.0 | 32.2 | 29.9 | 32.4 | 31.3 | 31.8 | 30.0 |
| | 0.01% PS80 | 6.2 | 30.5 | 29.0 | 30.9 | 29.9 | 30.2 | 29.9 |
| | | 6.5 | 31.6 | 32.0 | 32.4 | 33.6 | 32.4 | 31.5 |
| 20 mM | 75 mM NaCl, | 5.5 | 28.9 | 29.3 | 29.5 | 30.6 | 29.0 | 30.5 |
| Histidine | 125 mM | 5.8 | 30.8 | 31.3 | 31.4 | 31.6 | 31.3 | 31.4 |
| | Sucrose, | 6.0 | 30.9 | 28.6 | 31.1 | 30.9 | 30.4 | 30.3 |
| | 0.01% PS80 | 6.2 | 30.6 | 29.3 | 31.3 | 30.7 | 30.6 | 30.5 |
| | | 6.5 | 31.8 | 30.7 | 30.7 | 29.7 | 30.2 | 29.8 |

TABLE 15
pH values

| | Excipient/ | | Time Zero | | 3-week incubation | | | |
|---|---|---|---|---|---|---|---|---|
| Buffer | Surfactant | pH | 1 | 2 | 2-8° C. | | 50° C. | |
| 20 mM | 250 mM | 5.5 | 5.5 | 5.5 | 5.5 | 5.5 | 5.5 | 5.5 |
| Histidine | Sorbitol, | 5.8 | 5.7 | 5.8 | 5.8 | 5.8 | 5.8 | 5.7 |
| | 0.01% PS80 | 6.0 | 6.0 | 5.9 | 6.0 | 5.9 | 6.0 | 5.9 |
| | | 6.2 | 6.3 | 6.2 | 6.2 | 6.2 | 6.2 | 6.1 |
| | | 6.5 | 6.4 | 6.4 | 6.5 | 6.4 | 6.5 | 6.4 |
| 20 mM | 250 mM | 5.5 | 5.5 | 5.5 | 5.5 | 5.5 | 5.5 | 5.5 |
| Histidine | Sucrose, | 5.8 | 5.8 | 5.8 | 5.8 | 5.8 | 5.8 | 5.7 |
| | 0.01% PS80 | 6.0 | 6.0 | 5.9 | 6.0 | 6.0 | 6.0 | 6.0 |
| | | 6.2 | 6.2 | 6.2 | 6.1 | 6.1 | 6.1 | 6.1 |
| | | 6.5 | 6.4 | 6.5 | 6.4 | 6.5 | 6.4 | 6.4 |
| 20 mM | 75 mM NaCl, | 5.5 | 5.5 | 5.5 | 5.4 | 5.5 | 5.5 | 5.5 |
| Histidine | 125 mM | 5.8 | 5.8 | 5.8 | 5.8 | 5.7 | 5.7 | 5.7 |
| | Sorbitol, | 6.0 | 5.9 | 6.0 | 5.9 | 6.0 | 5.9 | 5.9 |
| | 0.01% PS80 | 6.2 | 6.2 | 6.2 | 6.2 | 6.1 | 6.1 | 6.1 |
| | | 6.5 | 6.5 | 6.5 | 6.5 | 6.5 | 6.5 | 6.4 |
| 20 mM | 75 mM NaCl, | 5.5 | 5.5 | 5.4 | 5.5 | 5.5 | 5.5 | 5.5 |
| Histidine | 125 mM | 5.8 | 5.8 | 5.9 | 5.8 | 5.7 | 5.8 | 5.7 |
| | Sucrose, | 6.0 | 6.0 | 5.9 | 6.0 | 5.9 | 6.0 | 5.9 |
| | 0.01% PS80 | 6.2 | 6.2 | 6.2 | 6.1 | 6.2 | 6.1 | 6.1 |
| | | 6.5 | 6.5 | 6.5 | 6.4 | 6.4 | 6.4 | 6.4 |

TABLE 16
Z-average values from DLS
Z-Average

| | Excipient/ | | Time Zero | | 3-week incubation | | | |
|---|---|---|---|---|---|---|---|---|
| Buffer | Surfactant | pH | 1 | 2 | 2-8° C. | | 50° C. | |
| 20 mM | 250 mM | 5.5 | 8.65 | 9.09 | 8.83 | 8.93 | 10.0 | 10.16 |
| Histidine | Sorbitol, | 5.8 | 8.86 | 8.54 | 8.80 | 8.62 | 9.52 | 9.46 |
| | 0.01% PS80 | 6.0 | 8.69 | 8.34 | 8.33 | 8.84 | 9.17 | 8.98 |
| | | 6.2 | 8.45 | 8.03 | 8.62 | 7.94 | 8.75 | 9.48 |
| | | 6.5 | 7.47 | 7.78 | 7.30 | 7.91 | 8.75 | 9.48 |
| 20 mM | 250 mM | 5.5 | 9.86 | 9.58 | 10.58 | 9.53 | 10.41 | 10.61 |
| Histidine | Sucrose, | 5.8 | 9.60 | 9.91 | 9.54 | 9.55 | 10.51 | 10.61 |
| | 0.01% PS80 | 6.0 | 9.24 | 9.23 | 9.30 | 9.55 | 10.64 | 10.05 |
| | | 6.2 | 8.90 | 8.80 | 8.96 | 9.12 | 10.11 | 10.26 |
| | | 6.5 | 8.14 | 7.86 | 8.67 | 8.44 | 10.06 | 9.49 |
| 20 mM | 75 mM NaCl, | 5.5 | 12.33 | 11.89 | 12.12 | 11.88 | 16.36 | 15.60 |
| Histidine | 125 mM | 5.8 | 12.55 | 11.98 | 12.13 | 12.03 | 14.22 | 14.01 |
| | Sorbitol, | 6.0 | 12.32 | 13.05 | 12.55 | 12.43 | 14.20 | 13.86 |
| | 0.01% PS80 | 6.2 | 12.42 | 13.10 | 12.81 | 12.79 | 13.61 | 13.81 |
| | | 6.5 | 12.87 | 12.85 | 13.06 | 12.59 | 14.11 | 13.44 |
| 20 mM | 75 mM NaCl, | 5.5 | 12.83 | 12.42 | 13.05 | 12.61 | 16.65 | 16.32 |
| Histidine | 125 mM | 5.8 | 12.89 | 12.68 | 13.19 | 12.65 | 15.61 | 14.68 |
| | Sucrose, | 6.0 | 13.33 | 12.72 | 13.11 | 12.92 | 14.67 | 14.25 |
| | 0.01% PS80 | 6.2 | 13.02 | 13.17 | 13.21 | 13.08 | 14.42 | 14.23 |
| | | 6.5 | 13.22 | 13.47 | 13.44 | 13.39 | 14.48 | 14.16 |

TABLE 17
PDI values from DLS
PDI

| | | | | | 3-week incubation | | | |
|---|---|---|---|---|---|---|---|---|
| | Excipient/ | | Time Zero | | 2-8° C. | | 50° C. | |
| Buffer | Surfactant | pH | 1 | 2 | 1 | 2 | 1 | 2 |
| 20 mM | 250 mM | 5.5 | 0.05 | 0.08 | 0.05 | 0.07 | 0.18 | 0.19 |
| Histidine | Sorbitol, | 5.8 | 0.05 | 0.01 | 0.06 | 0.08 | 0.15 | 0.14 |
| | 0.01% PS80 | 6.0 | 0.05 | 0.04 | 0.03 | 0.14 | 0.09 | 0.06 |
| | | 6.2 | 0.08 | 0.05 | 0.12 | 0.05 | 0.15 | 0.14 |
| | | 6.5 | 0.11 | 0.05 | 0.09 | 0.12 | 0.17 | 0.19 |
| 20 mM | 250 mM | 5.5 | 0.05 | 0.07 | 0.16 | 0.07 | 0.12 | 0.09 |
| Histidine | Sucrose, | 5.8 | 0.06 | 0.08 | 0.05 | 0.05 | 0.10 | 0.12 |
| | 0.01% PS80 | 6.0 | 0.07 | 0.08 | 0.07 | 0.11 | 0.18 | 0.12 |
| | | 6.2 | 0.05 | 0.05 | 0.08 | 0.10 | 0.09 | 0.15 |
| | | 6.5 | 0.08 | 0.06 | 0.16 | 0.19 | 0.18 | 0.14 |
| 20 mM | 75 mM NaCl, | 5.5 | 0.06 | 0.02 | 0.03 | 0.01 | 0.22 | 0.19 |
| Histidine | 125 mM | 5.8 | 0.05 | 0.03 | 0.03 | 0.01 | 0.13 | 0.15 |
| | Sorbitol, | 6.0 | 0.03 | 0.09 | 0.04 | 0.04 | 0.14 | 0.13 |
| | 0.01% PS80 | 6.2 | 0.02 | 0.12 | 0.06 | 0.07 | 0.12 | 0.11 |
| | | 6.5 | 0.05 | 0.03 | 0.07 | 0.03 | 0.10 | 0.06 |
| 20 mM | 75 mM NaCl, | 5.5 | 0.07 | 0.03 | 0.08 | 0.06 | 0.22 | 0.20 |
| Histidine | 125 mM | 5.8 | 0.04 | 0.03 | 0.08 | 0.03 | 0.18 | 0.15 |
| | Sucrose, | 6.0 | 0.06 | 0.03 | 0.06 | 0.06 | 0.12 | 0.12 |
| | 0.01% PS80 | 6.2 | 0.05 | 0.05 | 0.04 | 0.07 | 0.14 | 0.07 |
| | | 6.5 | 0.03 | 0.06 | 0.05 | 0.05 | 0.09 | 0.10 |

TABLE 18
Monomer % PD values
Monomer % Pd

| | | | | | 3-week incubation | | | |
|---|---|---|---|---|---|---|---|---|
| | Excipient/ | | Time Zero | | 2-8° C. | | 50° C. | |
| Buffer | Surfactant | pH | 1 | 2 | 1 | 2 | 1 | 2 |
| 20 mM | 250 mM | 5.5 | 27.0 | 30.9 | 27.7 | 30.8 | 35.0 | 37.0 |
| Histidine | Sorbitol, | 5.8 | 27.3 | 23.0 | 29.7 | 30.6 | 34.1 | 29.5 |
| | 0.01% PS80 | 6.0 | 27.9 | 26.2 | 25.1 | 29.8 | 30.9 | 26.5 |
| | | 6.2 | 31.0 | 27.5 | 33.8 | 26.3 | 36.4 | 30.6 |
| | | 6.5 | 35.6 | 27.0 | 33.9 | 33.1 | 38.0 | 36.9 |
| 20 mM | 250 mM | 5.5 | 28.1 | 31.9 | 39.5 | 31.1 | 36.4 | 31.7 |
| Histidine | Sucrose, | 5.8 | 29.2 | 30.6 | 28.4 | 28.6 | 33.8 | 35.3 |
| | 0.01% PS80 | 6.0 | 30.4 | 31.3 | 28.8 | 34.8 | 37.2 | 36.3 |
| | | 6.2 | 29.0 | 28.1 | 31.7 | 35.3 | 33.1 | 35.0 |
| | | 6.5 | 31.2 | 29.2 | 36.6 | 36.0 | 38.2 | 39.3 |
| 20 mM | 75 mM NaCl, | 5.5 | 28.7 | 23.6 | 24.6 | 23.0 | 43.8 | 30.7 |
| Histidine | 125 mM | 5.8 | 26.5 | 24.2 | 24.1 | 23.4 | 29.7 | 34.4 |
| | Sorbitol, | 6.0 | 25.1 | 32.1 | 25.9 | 24.1 | 35.1 | 33.0 |
| | 0.01% PS80 | 6.2 | 24.3 | 33.3 | 27.1 | 29.7 | 34.9 | 35.5 |
| | | 6.5 | 26.1 | 24.6 | 29.9 | 24.0 | 33.0 | 26.1 |
| 20 mM | 75 mM NaCl, | 5.5 | 29.3 | 24.7 | 29.6 | 26.5 | 45.7 | 30.8 |
| Histidine | 125 mM | 5.8 | 25.8 | 24.8 | 30.4 | 24.2 | 36.8 | 37.5 |
| | Sucrose, | 6.0 | 29.1 | 24.2 | 28.3 | 27.2 | 36.4 | 35.1 |
| | 0.01% PS80 | 6.2 | 26.6 | 26.8 | 25.7 | 27.5 | 36.2 | 24.5 |
| | | 6.5 | 25.6 | 29.3 | 27.1 | 27.3 | 32.4 | 32.5 |

TABLE 19
Monomer size values
Monomer Size (d.nm)

| | | | | | 3-week incubation | | | |
|---|---|---|---|---|---|---|---|---|
| | Excipient/ | | Time Zero | | 2-8° C. | | 50° C. | |
| Buffer | Surfactant | pH | 1 | 2 | 1 | 2 | 1 | 2 |
| 20 mM | 250 mM | 5.5 | 9.22 | 9.96 | 9.46 | 9.73 | 10.80 | 11.07 |
| Histidine | Sorbitol, | 5.8 | 9.47 | 8.89 | 9.51 | 9.42 | 10.43 | 10.13 |
| | 0.01% PS80 | 6.0 | 9.32 | 8.87 | 8.78 | 9.51 | 10.05 | 9.60 |
| | | 6.2 | 9.27 | 8.60 | 9.54 | 8.45 | 10.68 | 9.84 |
| | | 6.5 | 8.42 | 8.30 | 8.10 | 8.75 | 9.78 | 10.25 |
| 20 mM | 250 mM | 5.5 | 10.58 | 10.51 | 12.14 | 10.40 | 11.87 | 11.22 |
| Histidine | Sucrose, | 5.8 | 10.35 | 10.89 | 10.24 | 10.24 | 11.70 | 11.86 |
| | 0.01% PS80 | 6.0 | 10.11 | 10.17 | 10.10 | 10.82 | 11.70 | 11.47 |
| | | 6.2 | 9.58 | 9.42 | 9.85 | 10.24 | 11.20 | 11.30 |
| | | 6.5 | 8.93 | 8.48 | 9.72 | 9.11 | 11.25 | 10.90 |
| 20 mM | 75 mM NaCl, | 5.5 | 13.29 | 12.44 | 12.75 | 12.37 | 18.16 | 16.12 |
| Histidine | 125 mM | 5.8 | 13.40 | 12.61 | 12.75 | 12.56 | 15.37 | 15.33 |
| | Sorbitol, | 6.0 | 12.99 | 14.35 | 13.34 | 13.10 | 15.73 | 15.23 |
| | 0.01% PS80 | 6.2 | 13.03 | 14.51 | 13.72 | 13.90 | 15.24 | 15.55 |
| | | 6.5 | 13.70 | 13.54 | 14.15 | 13.22 | 15.69 | 14.33 |
| 20 mM | 75 mM NaCl, | 5.5 | 13.94 | 13.09 | 14.25 | 13.46 | 19.09 | 16.75 |
| Histidine | 125 mM | 5.8 | 13.70 | 13.37 | 14.39 | 13.28 | 17.15 | 16.43 |
| | Sucrose, | 6.0 | 14.40 | 13.39 | 14.11 | 13.86 | 16.70 | 15.96 |
| | 0.01% PS80 | 6.2 | 13.87 | 14.11 | 14.00 | 14.06 | 16.29 | 15.17 |
| | | 6.5 | 13.99 | 14.53 | 14.41 | 14.30 | 16.03 | 15.60 |

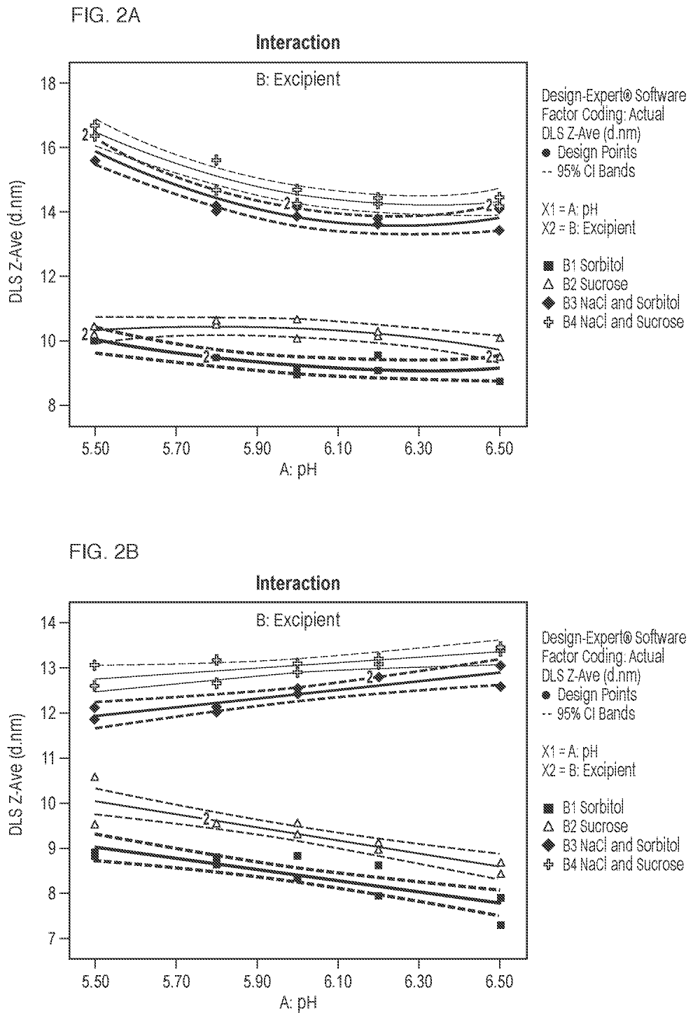

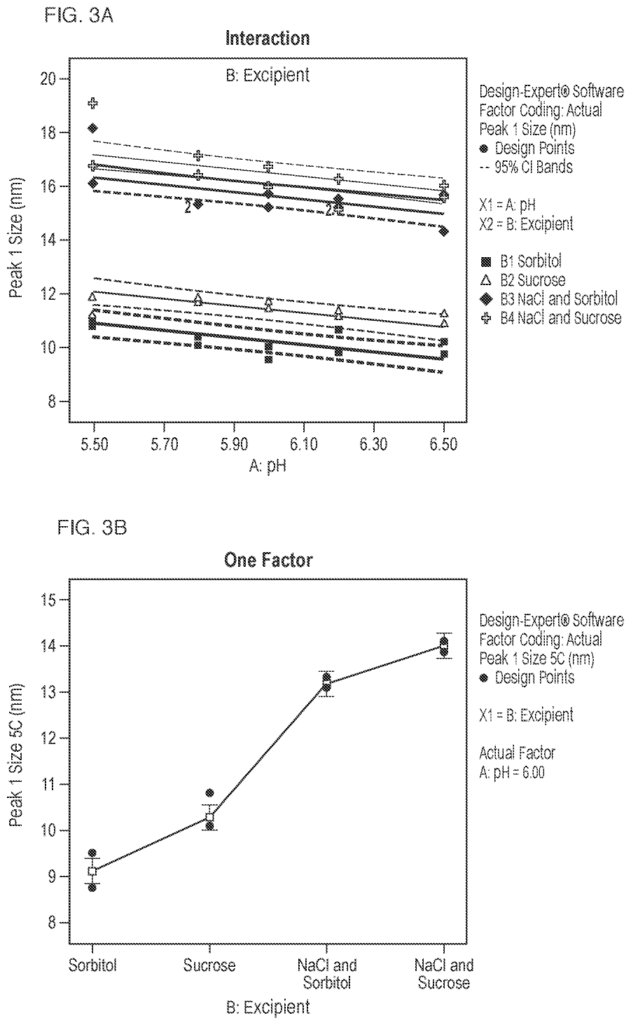

As evaluated by DLS, with average sizes and monomer sizes≤20 nm, and polydispersity (PDI)≤0.300, all A49-F3′-TriNKET-Trastuzumab formulations displayed conformational stability. In evaluating the excipient and pH combinations, sorbitol and sucrose only formulations had lower average sizes relative to the combination formulations NaCl and sorbitol and NaCl and sucrose upon incubation for 3 weeks at 2-8° C. and 50° C. (comparative modeling shown in FIG. 2A and FIG. 2B). Average monomer size was also lower in sorbitol and sucrose only formulations compared to the combination formulations with NaCl after incubation for 3 weeks at 2-8° C. and 50° C. (FIGS. 3A and 3B).

### Size Exclusion Chromatography (SEC)

Size exclusion chromatography was performed according to the draft method in order to determine the percentage of high molecular weight species (% HMW), percentage of main species (% Main), and percentage of low molecular weight species (% LMW). Samples were diluted to 2.0 mg/mL in mobile phase buffer (containing 100 mM phosphate, 150 mM sodium chloride pH 7.3) and injected at a 100 μg load. Separation was performed with a Tosoh G3000SWx1 (7.8×300 mm, cat. #08541) column with detection at 280 nm with 8 nm bandwidth. Samples were analyzed in real time, at time zero, and following a 3-week incubation at either 2-8° C. or 50° C. The % HMW values are summarized in Table 20, the % main values are summarized in Table 21, and the % LMW values are summarized in Table 22.

TABLE 20
% HMW SEC
% HMW

| | | | | | 3-week incubation | | | |
|---|---|---|---|---|---|---|---|---|
| | Excipient/ | | Time Zero | | 2-8° C. | | 50° C. | |
| Buffer | Surfactant | pH | 1 | 2 | 1 | 2 | 1 | 2 |
| 20 mM | 250 mM | 5.5 | 0.6 | 0.5 | 0.6 | 0.6 | 3.1 | 3.2 |
| Histidine | Sorbitol, | 5.8 | 0.6 | 0.6 | 0.6 | 0.6 | 2.9 | 2.9 |
| | 0.01% PS80 | 6.0 | 0.6 | 0.6 | 0.6 | 0.6 | 3.0 | 2.9 |
| | | 6.2 | 0.6 | 0.6 | 0.7 | 0.7 | 3.1 | 3.0 |
| | | 6.5 | 0.6 | 0.6 | 0.8 | 0.7 | 3.5 | 3.2 |
| | 250 mM | 5.5 | 0.5 | 0.5 | 0.6 | 0.6 | 3.2 | 2.8 |
| | Sucrose, | 5.8 | 0.5 | 0.6 | 0.6 | 0.6 | 3.1 | 3.0 |
| | 0.01% PS80 | 6.0 | 0.5 | 0.6 | 0.6 | 0.6 | 3.2 | 2.9 |
| | | 6.2 | 0.6 | 0.6 | 0.7 | 0.7 | 3.3 | 3.2 |
| | | 6.5 | 0.6 | 0.6 | 0.7 | 0.7 | 3.4 | 3.5 |
| | 75 mM NaCl, | 5.5 | 0.6 | 0.6 | 0.6 | 0.6 | 6.1 | 5.6 |
| | 125 mM | 5.8 | 0.6 | 0.6 | 0.7 | 0.7 | 4.7 | 4.8 |
| | Sorbitol, | 6.0 | 0.6 | 0.6 | 0.7 | 0.7 | 4.5 | 4.3 |
| | 0.01% PS80 | 6.2 | 0.6 | 0.6 | 0.7 | 0.7 | 4.2 | 4.1 |
| | | 6.5 | 0.6 | 0.7 | 0.8 | 0.8 | 4.3 | 4.3 |
| | 75 mM NaCl, | 5.5 | 0.6 | 0.5 | 0.6 | 0.6 | 5.9 | 5.7 |
| | 125 mM | 5.8 | 0.6 | 0.6 | 0.6 | 0.6 | 5.0 | 4.9 |
| | Sucrose, | 6.0 | 0.6 | 0.6 | 0.7 | 0.7 | 4.5 | 4.5 |
| | 0.01% PS80 | 6.2 | 0.6 | 0.6 | 0.7 | 0.7 | 4.3 | 4.1 |
| | | 6.5 | 0.6 | 0.6 | 0.8 | 0.7 | 4.0 | 4.1 |

TABLE 21
% Main Peak SEC
% Main Peak

| | | | | | 3-week incubation | | | |
|---|---|---|---|---|---|---|---|---|
| | Excipient/ | | Time Zero | | 2-8° C. | | 50° C. | |
| Buffer | Surfactant | pH | 1 | 2 | 1 | 2 | 1 | 2 |
| 20 mM | 250 mM | 5.5 | 99.2 | 99.2 | 98.9 | 99.0 | 94.6 | 94.7 |
| Histidine | Sorbitol, | 5.8 | 99.1 | 99.1 | 98.9 | 98.8 | 95.2 | 95.1 |
| | 0.01% PS80 | 6.0 | 99.1 | 99.1 | 98.9 | 98.8 | 95.1 | 95.2 |
| | | 6.2 | 99.1 | 99.0 | 98.8 | 98.8 | 95.1 | 95.2 |
| | | 6.5 | 99.1 | 99.1 | 98.8 | 98.8 | 94.6 | 94.9 |
| 20 mM | 250 mM | 5.5 | 99.2 | 99.1 | 98.9 | 98.8 | 94.5 | 95.0 |
| Histidine | Sucrose, | 5.8 | 99.1 | 99.1 | 98.8 | 98.7 | 94.7 | 94.9 |
| | 0.01% PS80 | 6.0 | 99.2 | 99.1 | 98.9 | 98.8 | 94.9 | 95.1 |
| | | 6.2 | 99.1 | 99.0 | 98.7 | 98.7 | 94.6 | 94.9 |
| | | 6.5 | 99.1 | 99.0 | 98.7 | 98.6 | 94.6 | 94.4 |
| 20 mM | 75 mM NaCl, | 5.5 | 99.1 | 99.2 | 98.9 | 99.0 | 91.3 | 92.0 |
| Histidine | 125 mM | 5.8 | 99.1 | 99.1 | 98.9 | 98.9 | 93.2 | 93.2 |
| | Sorbitol, | 6.0 | 99.2 | 99.0 | 98.9 | 98.9 | 93.5 | 93.7 |
| | 0.01% PS80 | 6.2 | 99.1 | 99.1 | 98.9 | 98.9 | 94.0 | 93.9 |
| | | 6.5 | 99.1 | 99.1 | 98.8 | 98.8 | 93.7 | 93.8 |
| 20 mM | 75 mM NaCl, | 5.5 | 99.1 | 99.2 | 98.9 | 99.0 | 91.3 | 91.9 |
| Histidine | 125 mM | 5.8 | 99.2 | 99.1 | 98.9 | 98.8 | 92.7 | 93.0 |
| | Sucrose, | 6.0 | 99.1 | 99.1 | 98.9 | 98.9 | 93.5 | 93.4 |
| | 0.01% PS80 | 6.2 | 99.0 | 99.1 | 98.9 | 98.9 | 93.6 | 93.8 |
| | | 6.5 | 99.1 | 99.1 | 98.8 | 98.8 | 93.9 | 94.0 |

TABLE 22
% LMW SEC
% LMW

| | | | | | 3-week incubation | | | |
|---|---|---|---|---|---|---|---|---|
| | Excipient/ | | Time Zero | | 2-8° C. | | 50° C. | |
| Buffer | Surfactant | pH | 1 | 2 | 1 | 2 | 1 | 2 |
| 20 mM | 250 mM | 5.5 | 0.3 | 0.3 | 0.5 | 0.5 | 2.3 | 2.2 |
| Histidine | Sorbitol, | 5.8 | 0.4 | 0.3 | 0.5 | 0.5 | 2.0 | 2.0 |
| | 0.01% PS80 | 6.0 | 0.3 | 0.3 | 0.5 | 0.6 | 1.9 | 1.8 |
| | | 6.2 | 0.3 | 0.4 | 0.5 | 0.5 | 1.8 | 1.8 |
| | | 6.5 | 0.2 | 0.3 | 0.4 | 0.5 | 1.9 | 1.9 |
| 20 mM | 250 mM | 5.5 | 0.3 | 0.3 | 0.6 | 0.6 | 2.3 | 2.2 |
| Histidine | Sucrose, | 5.8 | 0.4 | 0.4 | 0.6 | 0.7 | 2.2 | 2.1 |
| | 0.01% PS80 | 6.0 | 0.3 | 0.4 | 0.5 | 0.6 | 1.9 | 2.0 |
| | | 6.2 | 0.3 | 0.4 | 0.7 | 0.6 | 2.1 | 1.9 |
| | | 6.5 | 0.3 | 0.4 | 0.6 | 0.7 | 2.1 | 2.1 |
| 20 mM | 75 mM NaCl, | 5.5 | 0.4 | 0.3 | 0.5 | 0.4 | 2.6 | 2.4 |
| Histidine | 125 mM | 5.8 | 0.3 | 0.3 | 0.4 | 0.5 | 2.1 | 2.0 |
| | Sorbitol, | 6.0 | 0.2 | 0.4 | 0.4 | 0.4 | 2.0 | 2.0 |
| | 0.01% PS80 | 6.2 | 0.3 | 0.3 | 0.4 | 0.4 | 1.9 | 2.0 |
| | | 6.5 | 0.3 | 0.3 | 0.4 | 0.4 | 1.9 | 1.9 |
| 20 mM | 75 mM NaCl, | 5.5 | 0.3 | 0.3 | 0.5 | 0.4 | 2.8 | 2.4 |
| Histidine | 125 mM | 5.8 | 0.3 | 0.3 | 0.5 | 0.5 | 2.2 | 2.1 |
| | Sucrose, | 6.0 | 0.3 | 0.3 | 0.4 | 0.4 | 2.1 | 2.0 |
| | 0.01% PS80 | 6.2 | 0.4 | 0.3 | 0.4 | 0.4 | 2.0 | 2.1 |
| | | 6.5 | 0.3 | 0.3 | 0.5 | 0.5 | 2.1 | 1.9 |

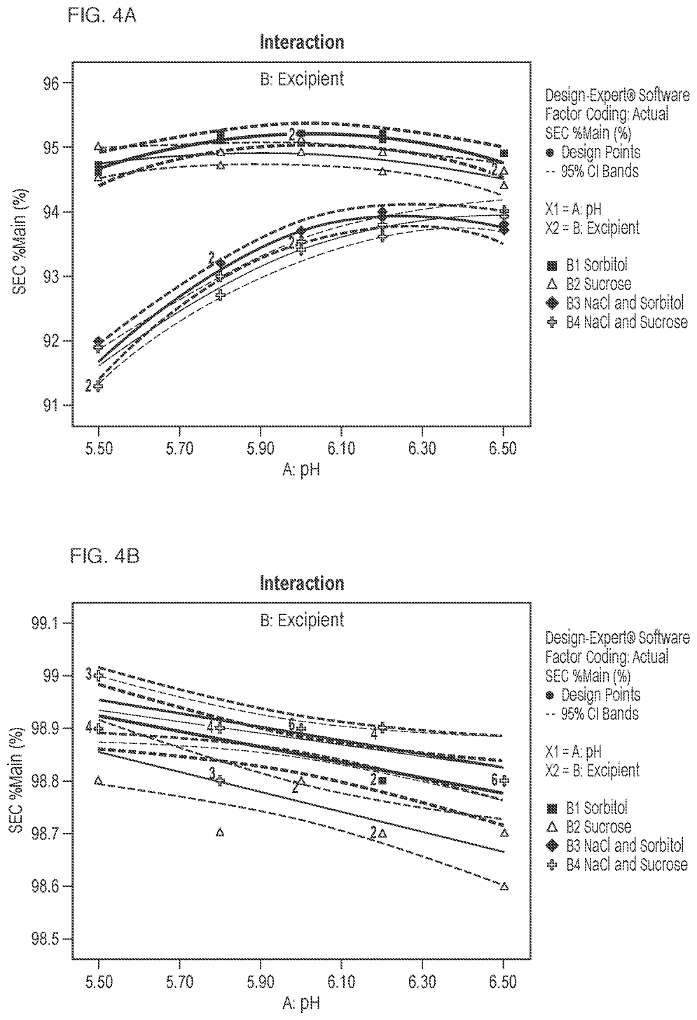

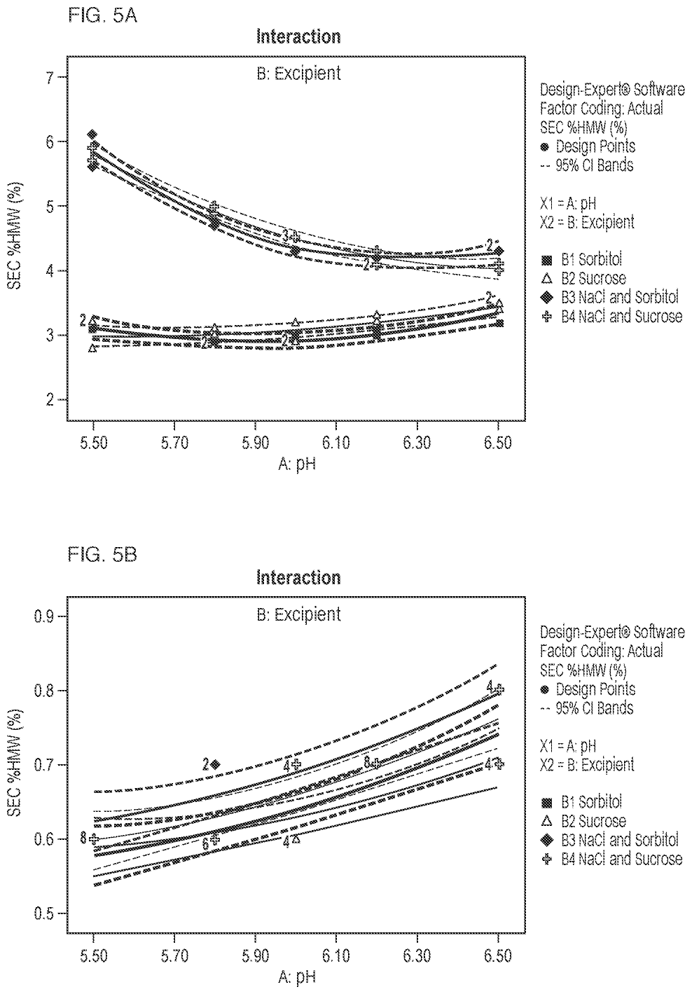

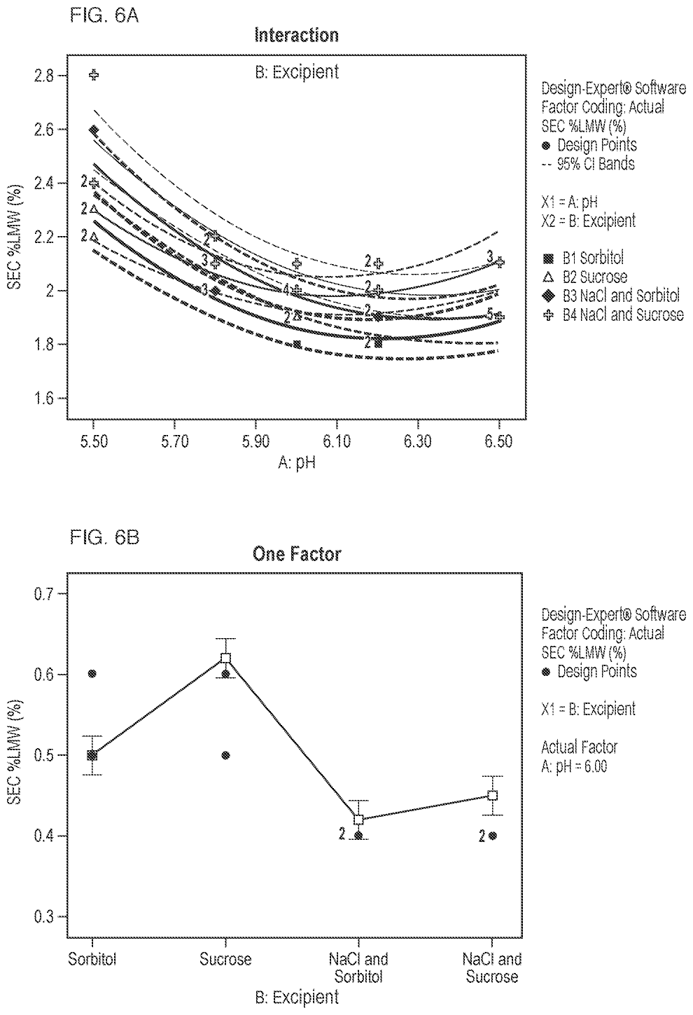

After 3-week incubation at 50° C., the formulated samples possessed %main peak values ranging from 91.3%-95.2%, with those formulations possessing only sorbitol and sucrose maintaining greater %main peak and lower % HMW species (shown respectively in FIG. 4A and FIG. 5A) relative to the combination excipients NaCl and sorbitol and NaCl and sucrose. Importantly, both single excipients sucrose and sorbitol maintained %main peak species across the pH range 5.5-6.5, and both display only slightly elevated % HMW species as the pH increased from 5.5 to 6.5. The % LMW species trended lower as the pH increased from 5.5 to about 6.2 for all excipients (FIG. 6A).

After 3-week incubation at 2-8° C., all the formulated samples maintained a percentage of main species peak greater than 98%. The % main peak was greater for lower pH values (5.5) versus higher pH values (pH 6.5) as shown in FIG. 4B. The single excipients sorbitol and sucrose trended towards lower % HMW relative to the combination excipients NaCl and sorbitol and NaCl and sucrose. For all excipients, increased pH trended towards increased % HMW species (FIG. 4B). The % LMW species trended lower for the combination excipients but was pH independent (FIG. 6B).

### Osmolality

The osmolality (osmo) of all samples was measured by freezing point depression. The osmolality was maintained for all samples across all conditions. The osmolality data at time zero, after 3-week incubation at 2-8° C., and after 3-week incubation at 50° C. are summarized in in Table 23.

TABLE 23
Osmolality values

| | | | | | 3-week incubation | | | |
|---|---|---|---|---|---|---|---|---|
| | Excipient/ | | Time Zero | | 2-8° C. | | 50° C. | |
| Buffer | Surfactant | pH | 1 | 2 | 1 | 2 | 1 | 2 |
| 20 mM | 250 mM | 5.5 | 314 | 319 | 309 | 315 | 309 | 317 |
| Histidine | Sorbitol, | 5.8 | 310 | 314 | 317 | 320 | 307 | 320 |
| | 0.01% PS80 | 6.0 | 309 | 299 | 311 | 303 | 313 | 301 |
| | | 6.2 | 304 | 351 | 304 | 354 | 308 | 354 |
| | | 6.5 | 304 | 302 | 297 | 298 | 301 | 300 |
| 20 mM | 250 mM | 5.5 | 343 | 347 | 342 | 348 | 354 | 360 |
| Histidine | Sucrose, | 5.8 | 349 | 352 | 350 | 351 | 362 | 364 |
| | 0.01% PS80 | 6.0 | 344 | 340 | 345 | 337 | 348 | 346 |
| | | 6.2 | 343 | 339 | 346 | 344 | 349 | 348 |
| | | 6.5 | 335 | 337 | 332 | 340 | 334 | 344 |
| 20 mM | 75 mM NaCl, | 5.5 | 322 | 326 | 321 | 330 | 322 | 330 |
| Histidine | 125 mM | 5.8 | 327 | 317 | 326 | 320 | 330 | 320 |
| | Sorbitol, | 6.0 | 323 | 372 | 317 | 376 | 320 | 375 |
| | 0.01% PS80 | 6.2 | 313 | 310 | 309 | 308 | 311 | 315 |
| | | 6.5 | 313 | 312 | 312 | 316 | 313 | 313 |
| 20 mM | 75 mM NaCl, | 5.5 | 348 | 331 | 345 | 338 | 354 | 343 |
| Histidine | 125 mM | 5.8 | 343 | 345 | 340 | 350 | 349 | 354 |
| | Sucrose, | 6.0 | 347 | 339 | 344 | 338 | 346 | 343 |
| | 0.01% PS80 | 6.2 | 341 | 335 | 337 | 338 | 343 | 337 |
| | | 6.5 | 342 | 339 | 332 | 343 | 339 | 342 |

Imaged Capillary Isoelectric Focusing (icIEF)

For determining charge-variant analysis, Imaged Capillary Isoelectric Focusing (icIEF) was used. Charge heterogeneity was evaluated using a draft method for the Protein Simple—Maurice. Starting Material and samples were diluted to 5 mg/mL in water then combined with master mix, 10 μL of sample to 90 μL of master mix. The master mix was a combination of 1% Methylcellulose, Pharmalyte 3-10, Pharmalyte 8-10.5, pI marker 5.12, pI marker 9.50, and DI water. A system suitability standard was prepared and run prior to running the samples, which were run in 96-well plate format. Method parameters utilized for the Maurice were as follows: focusing period #1=1 min, 1500 V, focusing period #2=8 min, 3000 V, detection=5 exposures, sample load=55 s, lower pI marker=5.12, 300 pixels, upper pI marker=9.50, 1800 pixels. Starting material was run every 18 injections to ensure consistent reads.

The “main peak” was identified as the main peak in the formulated samples at time zero. After incubation, the peak with the same elution time may have decreased and no longer represented the peak with the greatest area under curve, but was still identified as the “main peak.” The percentage of protein present in an acidic fraction (% acidic) values after 3-week incubation at 2-8° C. and after 3-week incubation at 50° C. are summarized in Table 24. The percentage of protein present in the main peak fraction (% main peak) values are summarized in Table 25. The percentage of protein present in a basic fraction (% basic) values are summarized in Table 26.

TABLE 24
% Acidic icIEF

| | | | 3-week incubation | | | |
|---|---|---|---|---|---|---|
| | Excipient/ | | 2-8° C. | | 50° C. | |
| Buffer | Surfactant | pH | 1 | 2 | 1 | 2 |
| 20 mM | 250 mM Sorbitol, | 5.5 | 35.8 | 36.0 | 73.1 | 73.3 |
| Histidine | 0.01% PS80 | 5.8 | 34.3 | 36.2 | 74.5 | 75.0 |
| | | 6.0 | 36.0 | 36.2 | 76.1 | 75.5 |
| | | 6.2 | 36.2 | 36.9 | 77.0 | 75.8 |
| | | 6.5 | 36.7 | 35.7 | 81.1 | 80.4 |
| 20 mM | 250 mM Sucrose, | 5.5 | 34.5 | 36.5 | 81.6 | 80.5 |
| Histidine | 0.01% PS80 | 5.8 | 35.0 | 36.5 | 81.4 | 80.8 |
| | | 6.0 | 34.7 | 34.5 | 79.0 | 78.4 |
| | | 6.2 | 36.1 | 36.4 | 79.8 | 80.1 |
| | | 6.5 | 36.5 | 37.2 | 81.0 | 81.5 |
| 20 mM | 75 mM NaCI, | 5.5 | 36.1 | 36.3 | 70.6 | 70.8 |
| Histidine | 125 mM Sorbitol, | 5.8 | 35.1 | 36.4 | 73.7 | 73.1 |
| | 0.01% PS80 | 6.0 | 36.3 | 35.6 | 75.0 | 74.2 |
| | | 6.2 | 35.3 | 35.2 | 76.4 | 76.3 |
| | | 6.5 | 36.4 | 35.5 | 78.9 | 78.6 |
| | | 5.5 | 35.0 | 36.1 | 75.7 | 74.5 |
| | 75 mM NaCI, | 5.8 | 35.3 | 36.4 | 77.0 | 75.9 |
| 20 mM | 125 mM Sucrose, | 6.0 | 36.4 | 36.6 | 78.0 | 77.4 |
| Histidine | 0.01% PS80 | 6.2 | 35.0 | 36.9 | 78.1 | 76.5 |
| | | 6.5 | 38.4 | 36.8 | 79.6 | 78.5 |

TABLE 25
% Main Peak icIEF

| | | | 3-week incubation | | | |
|---|---|---|---|---|---|---|
| | Excipient/ | | 2-8° C. | | 50° C. | |
| Buffer | Surfactant | pH | 1 | 2 | 1 | 2 |
| 20 mM | 250 mM Sorbitol, | 5.5 | 60.8 | 60.4 | 21.5 | 21.8 |
| Histidine | 0.01% PS80 | 5.8 | 62.2 | 60.2 | 21.4 | 21.0 |
| | | 6.0 | 60.3 | 60.3 | 20.4 | 20.9 |
| | | 6.2 | 60.4 | 59.5 | 19.8 | 20.8 |
| | | 6.5 | 59.9 | 60.9 | 16.1 | 17.2 |
| 20 mM | 250 mM Sucrose, | 5.5 | 62.0 | 59.9 | 14.9 | 16.0 |
| Histidine | 0.01% PS80 | 5.8 | 61.6 | 59.8 | 15.6 | 16.0 |
| | | 6.0 | 61.9 | 62.0 | 17.7 | 18.3 |
| | | 6.2 | 60.5 | 60.1 | 17.5 | 17.0 |
| | | 6.5 | 60.2 | 59.3 | 16.3 | 15.7 |
| 20 mM | 75 mM NaCl, 125 | 5.5 | 60.4 | 60.2 | 20.5 | 22.7 |
| Histidine | mM Sorbitol, | 5.8 | 61.4 | 60.1 | 21.8 | 22.1 |
| | 0.01% PS80 | 6.0 | 60.3 | 60.9 | 20.8 | 22.0 |
| | | 6.2 | 61.4 | 61.4 | 19.9 | 20.4 |
| | | 6.5 | 60.2 | 61.2 | 18.2 | 18.4 |
| 20 mM | 75 mM NaCl, 125 | 5.5 | 61.5 | 60.4 | 18.9 | 20.4 |
| Histidine | mM Sucrose, | 5.8 | 61.2 | 60.1 | 19.0 | 20.1 |
| | 0.01% PS80 | 6.0 | 60.2 | 59.9 | 18.7 | 18.9 |
| | | 6.2 | 61.6 | 59.6 | 18.7 | 20.3 |
| | | 6.5 | 58.3 | 59.7 | 17.6 | 18.4 |

TABLE 26
% Basic Peak icIEF

| | | | 3-week incubation | | | |
|---|---|---|---|---|---|---|
| | Excipient/ | | 2-8° C. | | 50° C. | |
| Buffer | Surfactant | pH | 1 | 2 | 1 | 2 |
| 20 mM | 250 mM Sorbitol | 5.5 | 3.4 | 3.6 | 5.4 | 4.9 |
| Histidine | 0.01% PS80 | 5.8 | 3.5 | 3.5 | 4.1 | 3.9 |
| | | 6.0 | 3.6 | 3.5 | 3.5 | 3.7 |
| | | 6.2 | 3.4 | 3.6 | 3.2 | 3.4 |
| | | 6.5 | 3.3 | 3.3 | 2.8 | 2.4 |
| 20 mM | 250 mM Sucrose, | 5.5 | 3.5 | 3.6 | 3.6 | 3.5 |
| Histidine | 0.01% PS80 | 5.8 | 3.4 | 3.7 | 3.0 | 3.2 |
| | | 6.0 | 3.4 | 3.5 | 3.3 | 3.3 |
| | | 6.2 | 3.4 | 3.5 | 2.7 | 2.9 |
| | | 6.5 | 3.4 | 3.5 | 2.7 | 2.8 |
| 20 mM | 75 mM NaCl, | 5.5 | 3.6 | 3.5 | 8.8 | 6.5 |
| Histidine | 125 mM Sorbitol, | 5.8 | 3.5 | 3.5 | 4.5 | 4.8 |
| | 0.01% PS80 | 6.0 | 3.4 | 3.5 | 4.3 | 3.8 |
| | | 6.2 | 3.4 | 3.4 | 3.7 | 3.3 |
| | | 6.5 | 3.4 | 3.3 | 2.9 | 3.0 |
| 20 mM | 75 mM NaCl, | 5.5 | 3.5 | 3.5 | 5.4 | 5.1 |
| Histidine | 125 mM Sucrose, | 5.8 | 3.5 | 3.5 | 4.0 | 4.0 |
| | 0.01% PS80 | 6.0 | 3.4 | 3.4 | 3.3 | 3.7 |
| | | 6.2 | 3.4 | 3.5 | 3.2 | 3.2 |
| | | 6.5 | 3.3 | 3.5 | 2.8 | 3.0 |

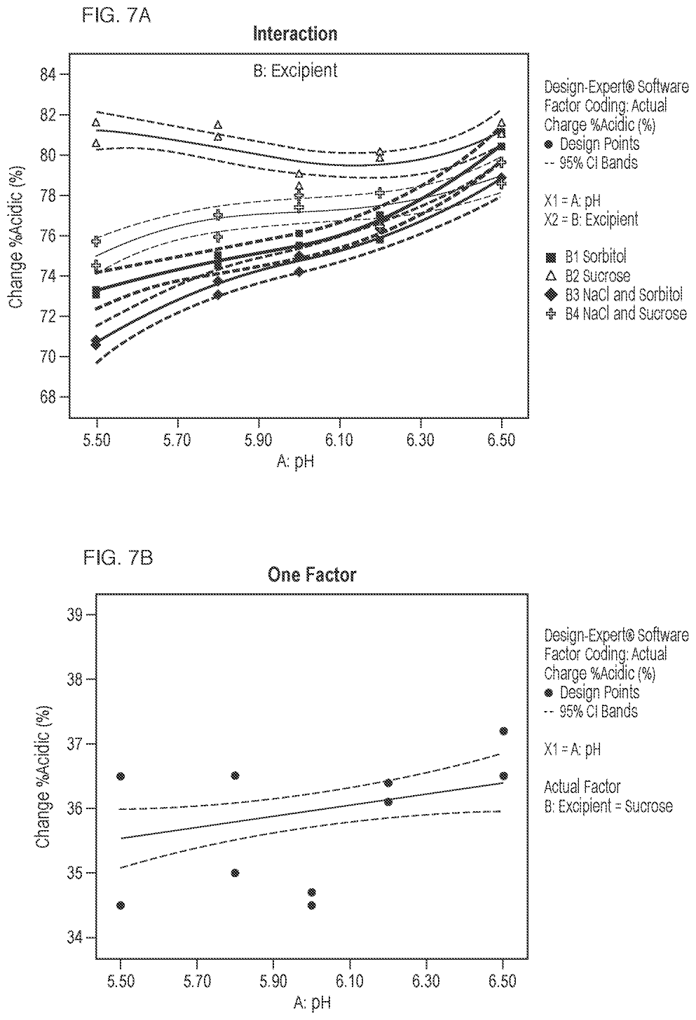

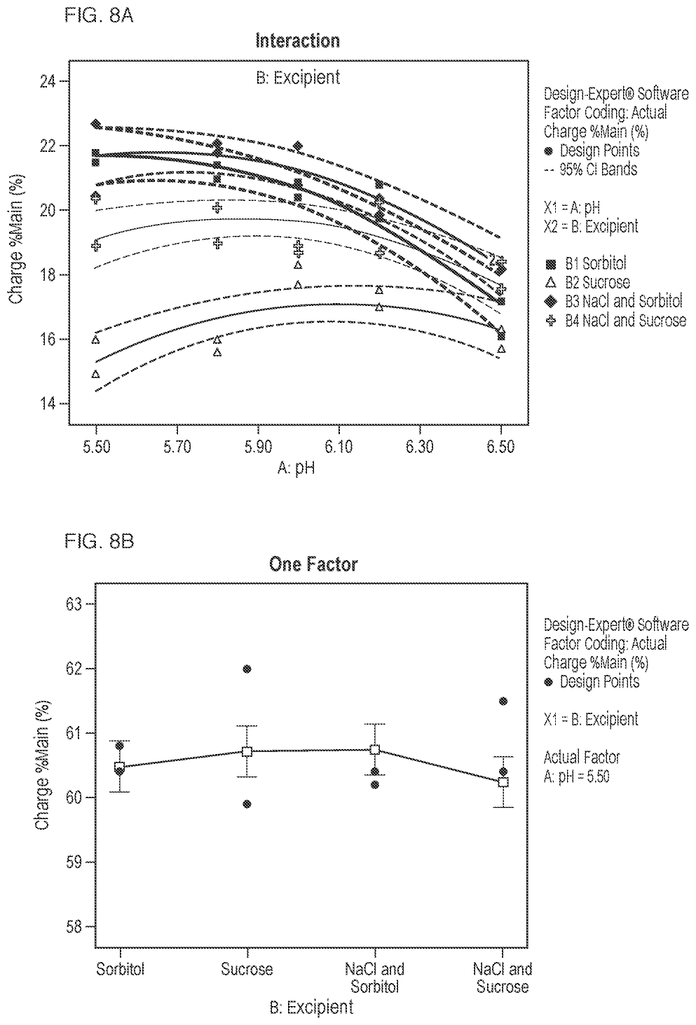

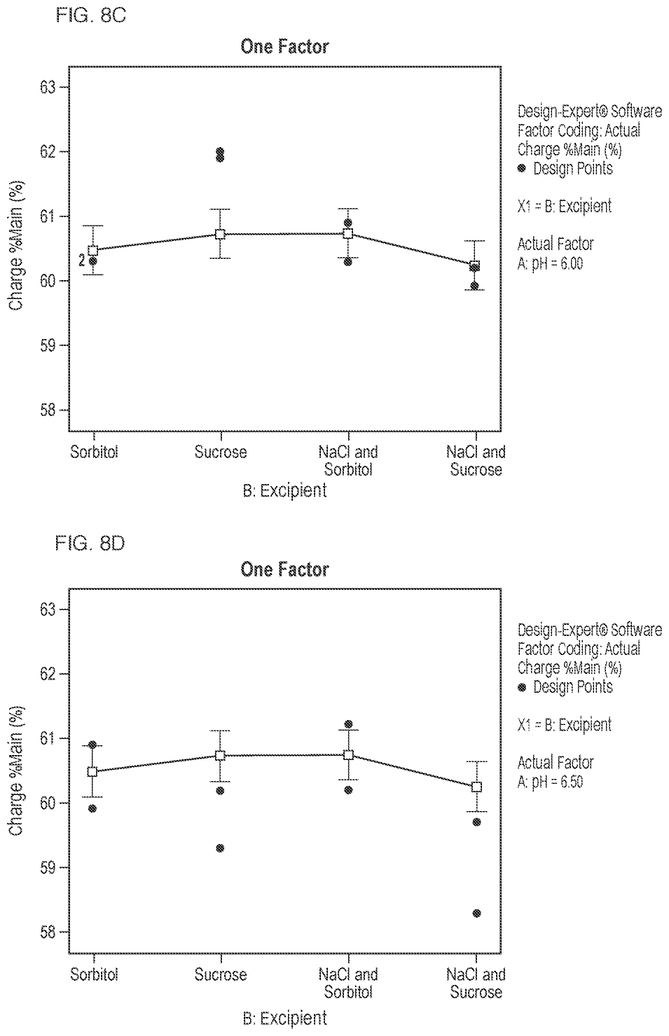

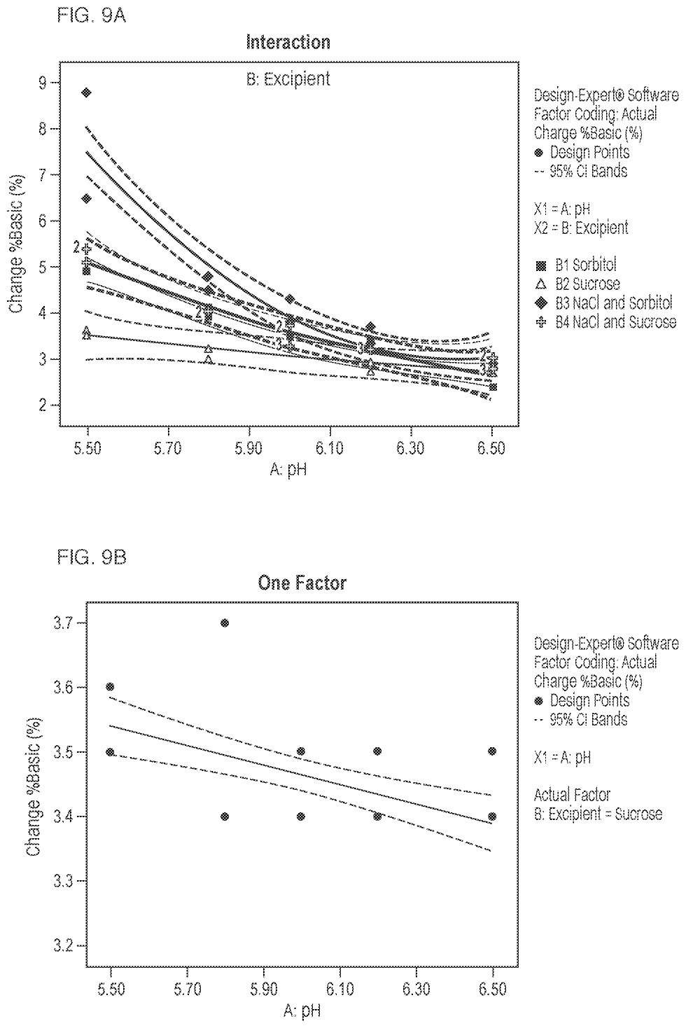

For the formulated samples after 3-week incubation at 2-8° C., the % main peak values ranged from 58.3%-62.2%, the % acidic values ranged from 34.3%-38.4%, and the % basic values ranged from 3.3%-3.7%. There was not a significant model to fit the % main peak data, indicating that neither pH nor excipient had a significant effect on the icIEF values (FIGS. 8B-8D). The excipient was also not significant in modeling the % acidic and % basic species, and the % acidic species tended to increase with pH while the % basic species decreased (FIGS. 7B and 9B). The changes were marginal, however, as observed by the narrow ranges for all values.

For the formulated samples after 3-week incubation at 50° C., the % main peak values ranged from 14.9%-22.7%, the % acidic values ranged from 70.6%-81.6%, and the % basic values ranged from 2.4%-8.8%. The data indicates a shift from the % main peak species to % acidic species, with % basic remaining relatively consistent with the 3-week 2-8° C. incubation results. In evaluating the 3-week 50° C. formulated samples, the samples possessing sucrose as the only excipient possessed the highest % acidic species (FIG. 7A) and lowest % main peak and % basic species (FIGS. 8A and 9A). This was consistent across all pH values (5.5-6.5) while the formulations possessing the other 3 excipients trended towards lower % acidic species at lower pH values that increased with increasing pH.

### Capillary Electrophoresis (CE)

Reduced capillary gel electrophoresis was performed to assess purity. SDS-CGE was evaluated per the draft ATM, using a Sciex PA800+ with UV detection at 220 nm. Samples were prepared by diluting 100 μg sample in Beckman SDS sample buffer and adding 5 μL β-Mercaptoethanol. Samples were heated at 70° C. for 10 minutes. Separation occurred over 20 minutes using normal polarity, 1 minute ramp, 15 kV voltage and 20 psi pressure. The capillary length was 30.2 cm, with the length to the detector as 10.2 cm. Starting material was used as a reference. A summary of sample percentage purity is shown in Table 27. A summary of percentage of sample impurities is shown in Table 28.

TABLE 27
% Purity

| | | | 3-week incubation | | | |
|---|---|---|---|---|---|---|
| | Excipient/ | | 2-8° C. | | 50° C. | |
| Buffer | Surfactant | pH | 1 | 2 | 1 | 2 |
| 20 mM | 250 mM Sorbitol, | 5.5 | 98.8 | 98.7 | 91.3 | 92.8 |
| Histidine | 0.01% PS80 | 5.8 | 98.9 | 98.7 | 92.9 | 92.7 |
| | | 6.0 | 98.8 | 98.2 | 94.1 | 93.3 |
| | | 6.2 | 98.7 | 98.6 | 93.5 | 93.4 |
| | | 6.5 | 98.9 | 98.8 | 92.2 | 92.5 |
| 20 mM | 250 mM Sucrose, | 5.5 | 99.0 | 98.8 | 91.5 | 93.1 |
| Histidine | 0.01% PS80 | 5.8 | 98.8 | 98.5 | 93.5 | 93.9 |
| | | 6.0 | 98.9 | 98.7 | 94.0 | 93.8 |
| | | 6.2 | 98.9 | 98.7 | 92.6 | 93.0 |
| | | 6.5 | 98.7 | 98.7 | 93.0 | 92.2 |
| 20 mM | 75 mM NaCl, 125 mM | 5.5 | 99.1 | 98.7 | 89.9 | 89.8 |
| Histidine | Sorbitol, 0.01% PS80 | 5.8 | 98.7 | 98.7 | 90.6 | 92.2 |
| | | 6.0 | 98.0 | 98.5 | 92.9 | 93.2 |
| | | 6.2 | 98.7 | 99.1 | 94.7 | 93.5 |
| | | 6.5 | 98.9 | 99.0 | 92.9 | 93.2 |
| 20 mM | 75 mM NaCl, 125 mM | 5.5 | 98.7 | 98.5 | 91.1 | 89.8 |
| Histidine | Sucrose, 0.01% PS80 | 5.8 | 98.8 | 99.1 | 93.9 | 92.8 |
| | | 6.0 | 99.0 | 99.2 | 93.1 | 92.7 |
| | | 6.2 | 99.0 | 98.7 | 93.3 | 92.9 |
| | | 6.5 | 97.2 | 98.9 | 93.9 | 94.3 |

TABLE 28
% Impurities

| | | | 3-week incubation | | | |
|---|---|---|---|---|---|---|
| | Excipient/ | | 2-8° C. | | 50° C. | |
| Buffer | Surfactant | pH | 1 | 2 | 1 | 2 |
| 20 mM | 250 mM Sorbitol, | 5.5 | 1.2 | 1.3 | 8.7 | 7.2 |
| Histidine | 0.01% PS80 | 5.8 | 1.1 | 1.3 | 7.1 | 7.3 |
| | | 6.0 | 1.2 | 1.8 | 5.9 | 6.7 |
| | | 6.2 | 1.3 | 1.4 | 6.5 | 6.6 |
| | | 6.5 | 1.1 | 1.2 | 7.8 | 7.5 |
| 20 mM | 250 mM Sucrose, | 5.5 | 1.0 | 1.2 | 8.5 | 6.9 |
| Histidine | 0.01% PS80 | 5.8 | 1.2 | 1.5 | 6.5 | 6.1 |
| | | 6.0 | 1.1 | 1.3 | 6.0 | 6.2 |
| | | 6.2 | 1.1 | 1.3 | 7.4 | 7.0 |
| | | 6.5 | 1.3 | 1.3 | 7.0 | 7.8 |
| 20 mM | 75 mM NaCl, 125 mM | 5.5 | 0.9 | 1.3 | 10.1 | 10.2 |
| Histidine | Sorbitol, 0.01% PS80 | 5.8 | 1.3 | 1.3 | 9.4 | 7.8 |
| | | 6.0 | 2.0 | 1.5 | 7.1 | 6.8 |
| | | 6.2 | 1.3 | 0.9 | 5.3 | 6.5 |
| | | 6.5 | 1.1 | 1.0 | 7.1 | 6.8 |
| 20 mM | 75 mM NaCl, 125 mM | 5.5 | 1.3 | 1.5 | 8.9 | 10.2 |
| Histidine | Sucrose, 0.01% PS80 | 5.8 | 1.2 | 0.9 | 6.1 | 7.2 |
| | | 6.0 | 1.0 | 0.8 | 6.9 | 7.3 |
| | | 6.2 | 1.0 | 1.3 | 6.7 | 7.1 |
| | | 6.5 | 2.8 | 1.1 | 6.1 | 5.7 |

Non-reduced capillary gel electrophoresis was also performed to assess purity. SDS-CGE was evaluated per the draft ATM, using a Sciex PA800+ with UV detection at 220 nm. Samples were prepared by diluting 100 μg sample in Beckman SDS sample buffer and adding 5 μL of 250 mM iodoacetamide. Samples were heated at 70° C. for 10 minutes. Separation occurred over 20 minutes using normal polarity, 1 minute ramp, 15 kV voltage and 20 psi pressure. The capillary length was 30.2 cm, with the length to the detector as 10.2 cm. Starting material was used as a reference. A summary of the % HMW CE (NR) data is shown in Table 29. A summary of the % main peak CE (NR) data is shown in Table 30. A summary of the % LMW CE (NR) data is shown in Table 31.

TABLE 29
% HMW CE (NR)

| | | | 3-week incubation | | | |
|---|---|---|---|---|---|---|
| | | | 2-8° C. | | 50° C. | |
| Buffer | Excipient/Surfactant | pH | 1 | 2 | 1 | 2 |
| 20 mM | 250 mM Sorbitol, 0.01% | 5.5 | 1.2 | 1.3 | 1.4 | 2.0 |
| Histidine | PS80 | 5.8 | 1.3 | 1.1 | 2.0 | 1.1 |
| | | 6.0 | 1.1 | 0.8 | 2.1 | 1.8 |
| | | 6.2 | 1.0 | 1.1 | 2.3 | 1.8 |
| | | 6.5 | 0.9 | 1.0 | 2.6 | 2.3 |
| 20 mM | 250 mM Sucrose, 0.01% | 5.5 | 1.3 | 1.1 | 1.4 | 1.1 |
| Histidine | PS80 | 5.8 | 1.0 | 1.2 | 2.0 | 1.8 |
| | | 6.0 | 1.1 | 0.9 | 2.3 | 2.2 |
| | | 6.2 | 1.0 | 0.9 | 2.5 | 2.1 |
| | | 6.5 | 0.8 | 0.6 | 2.3 | 2.0 |
| 20 mM | 75 mM NaCl, 125 mM | 5.5 | 1.3 | 1.5 | 2.1 | 1.5 |
| Histidine | Sorbitol, 0.01% PS80 | 5.8 | 0.6 | 1.5 | 2.4 | 1.8 |
| | | 6.0 | 1.2 | 1.3 | 2.1 | 2.1 |
| | | 6.2 | 0.9 | 1.2 | 3.0 | 2.6 |
| | | 6.5 | 1.2 | 1.2 | 3.0 | 3.2 |
| 20 mM | 75 mM NaCl, 125 mM | 5.5 | 1.4 | 1.3 | 2.3 | 1.6 |
| Histidine | Sucrose, 0.01% PS80 | 5.8 | 1.0 | 1.2 | 2.5 | 2.4 |
| | | 6.0 | 1.1 | 1.0 | 2.7 | 2.2 |
| | | 6.2 | 1.0 | 1.4 | 2.7 | 2.3 |
| | | 6.5 | 1.1 | 1.1 | 2.7 | 2.8 |

TABLE 30
% Main Peak CE (NR)

| | | | 3-week incubation | | | |
|---|---|---|---|---|---|---|
| | | | 2-8° C. | | 50° C. | |
| Buffer | Excipient/Surfactant | pH | 1 | 2 | 1 | 2 |
| 20 mM | 250 mM Sorbitol, 0.01% | 5.5 | 96.1 | 95.9 | 87.7 | 87.6 |
| Histidine | PS80 | 5.8 | 95.8 | 95.9 | 90.0 | 90.6 |
| | | 6.0 | 95.9 | 96.1 | 88.3 | 90.4 |
| | | 6.2 | 95.9 | 95.6 | 88.3 | 90.8 |
| | | 6.5 | 95.7 | 95.6 | 89.4 | 88.1 |
| 20 mM | 250 mM Sucrose, 0.01% | 5.5 | 95.7 | 95.9 | 90.0 | 90.7 |
| Histidine | PS80 | 5.8 | 95.7 | 95.9 | 88.4 | 89.3 |
| | | 6.0 | 95.9 | 96.2 | 89.6 | 88.9 |
| | | 6.2 | 95.9 | 96.0 | 89.6 | 90.3 |
| | | 6.5 | 95.8 | 95.9 | 88.5 | 90.4 |
| 20 mM | 75 mM NaCl, 125 mM | 5.5 | 95.7 | 95.8 | 88.1 | 88.8 |
| Histidine | Sorbitol, 0.01% PS80 | 5.8 | 95.7 | 95.5 | 87.5 | 89.7 |
| | | 6.0 | 95.5 | 95.7 | 88.1 | 89.7 |
| | | 6.2 | 95.9 | 95.7 | 88.0 | 87.8 |
| | | 6.5 | 95.6 | 95.9 | 87.6 | 87.4 |
| 20 mM | 75 mM NaCl, 125 mM | 5.5 | 95.3 | 95.7 | 87.9 | 88.6 |
| Histidine | Sucrose, 0.01% PS80 | 5.8 | 95.8 | 95.8 | 88.9 | 87.5 |
| | | 6.0 | 95.8 | 95.7 | 87.7 | 89.6 |
| | | 6.2 | 95.7 | 95.3 | 89.0 | 89.9 |
| | | 6.5 | 95.4 | 95.5 | 87.9 | 88.3 |

TABLE 31
% LMW CE (NR)

| | | | 3-week incubation | | | |
|---|---|---|---|---|---|---|
| | | | 2-8° C. | | 50° C. | |
| Buffer | Excipient/Surfactant | pH | 1 | 2 | 1 | 2 |
| 20 mM | 250 mM Sorbitol, 0.01% | 5.5 | 2.7 | 2.8 | 10.9 | 10.5 |
| Histidine | PS80 | 5.8 | 2.9 | 3.1 | 7.9 | 8.2 |
| | | 6.0 | 3.1 | 3.1 | 9.6 | 7.8 |
| | | 6.2 | 3.1 | 3.3 | 9.4 | 7.3 |
| | | 6.5 | 3.4 | 3.4 | 7.9 | 9.5 |
| 20 mM | 250 mM Sucrose, 0.01% | 5.5 | 2.9 | 2.9 | 8.6 | 8.2 |
| Histidine | PS80 | 5.8 | 3.3 | 2.9 | 9.5 | 8.9 |
| | | 6.0 | 2.9 | 3.0 | 8.1 | 8.9 |
| | | 6.2 | 3.1 | 3.1 | 7.9 | 7.6 |
| | | 6.5 | 3.5 | 3.5 | 9.3 | 7.6 |
| 20 mM | 75 mM NaCl, 125 mM | 5.5 | 3.0 | 2.8 | 9.8 | 9.7 |
| Histidine | Sorbitol, 0.01% PS80 | 5.8 | 3.7 | 3.0 | 10.1 | 8.5 |
| | | 6.0 | 3.3 | 3.0 | 9.8 | 8.2 |
| | | 6.2 | 3.3 | 3.1 | 9.0 | 9.6 |
| | | 6.5 | 3.3 | 2.8 | 9.3 | 9.4 |
| 20 mM | 75 mM NaCl, 125 mM | 5.5 | 3.3 | 3.1 | 9.8 | 9.8 |
| Histidine | Sucrose, 0.01% PS80 | 5.8 | 3.1 | 3.0 | 8.6 | 10.1 |
| | | 6.0 | 3.1 | 3.2 | 9.6 | 8.2 |
| | | 6.2 | 3.3 | 3.3 | 8.3 | 7.8 |
| | | 6.5 | 3.5 | 3.4 | 9.3 | 8.9 |

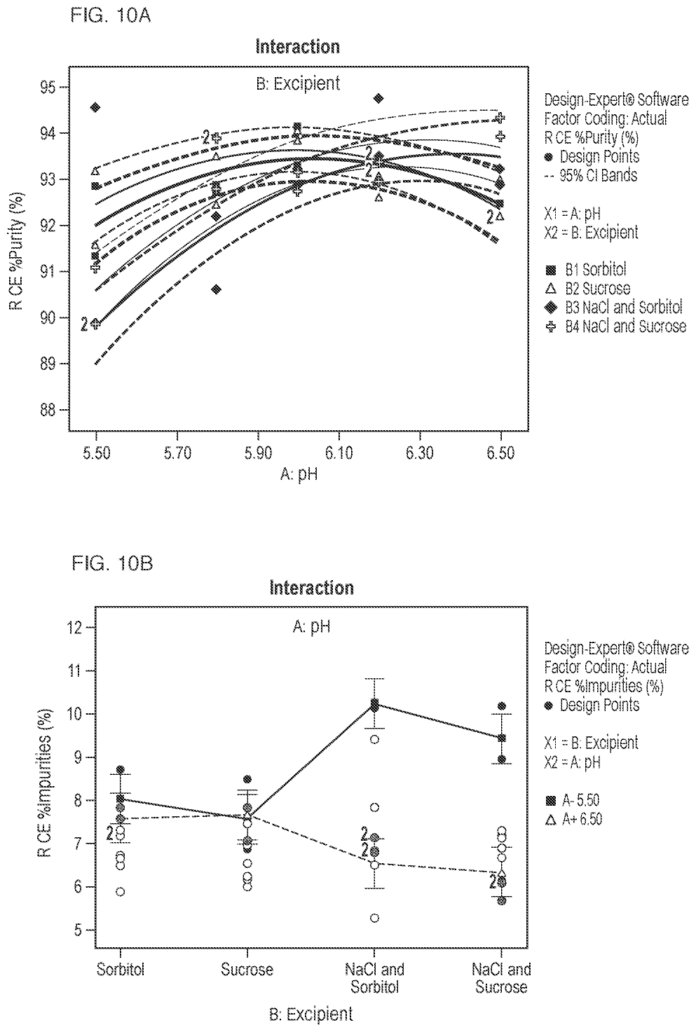

As evaludated by reduced CE, among the 3-week 50° C. samples, the % purity values for the sorbitol only and sucrose only formulations were maintained across the pH range (pH 5.5-6.5) (FIG. 10A), while the combination excipients displayed more variability regarding pH in that the % purity was reduced at lower pH values (5.5 versus 6.5) (FIG. 10B).

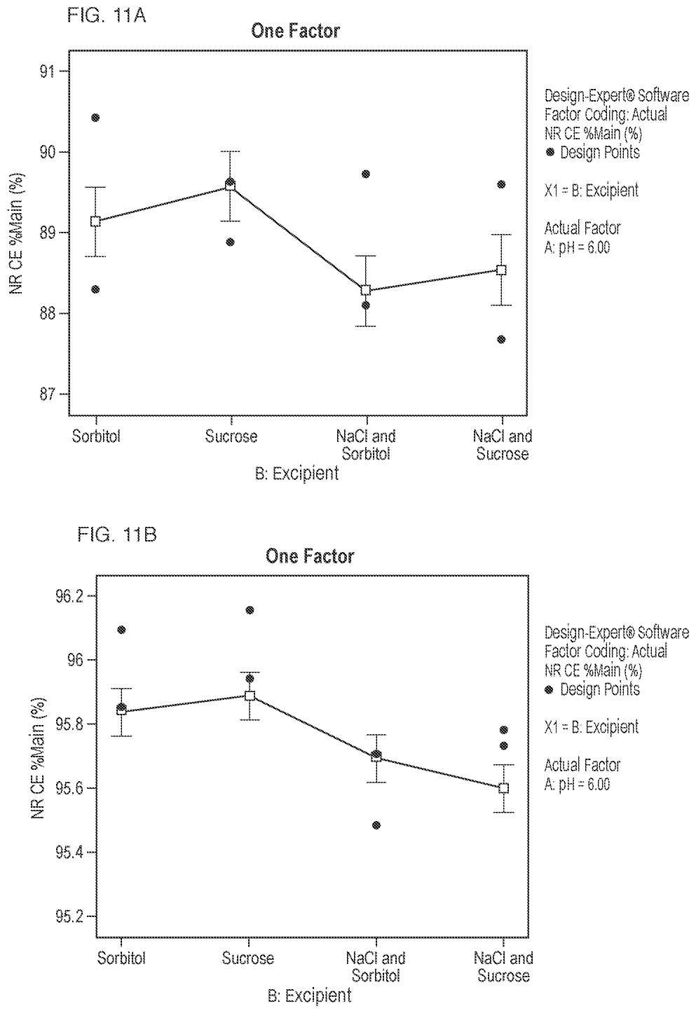

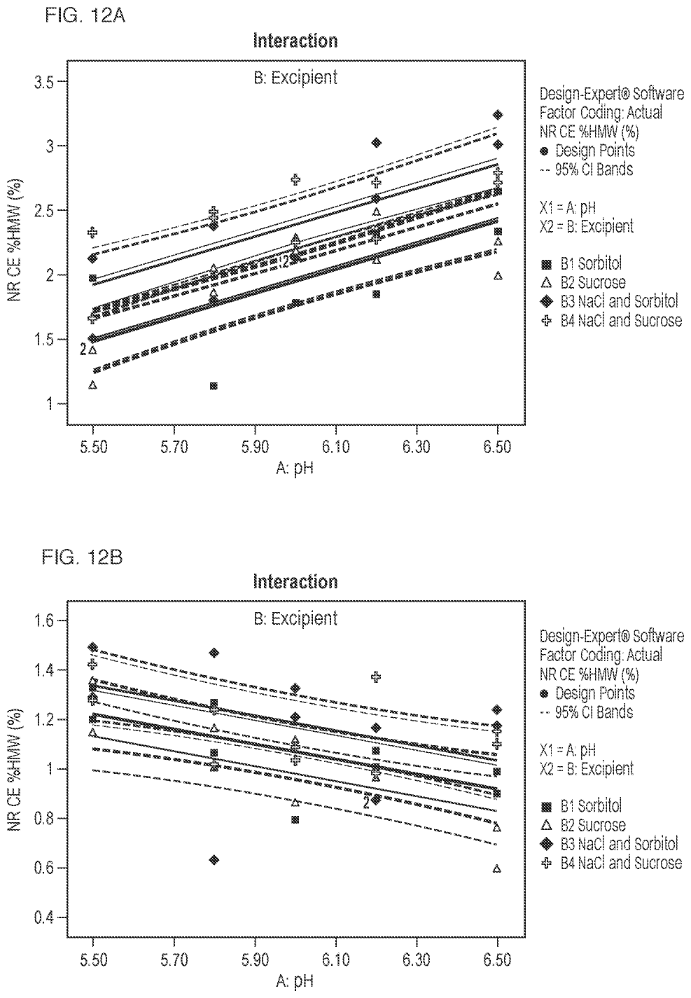

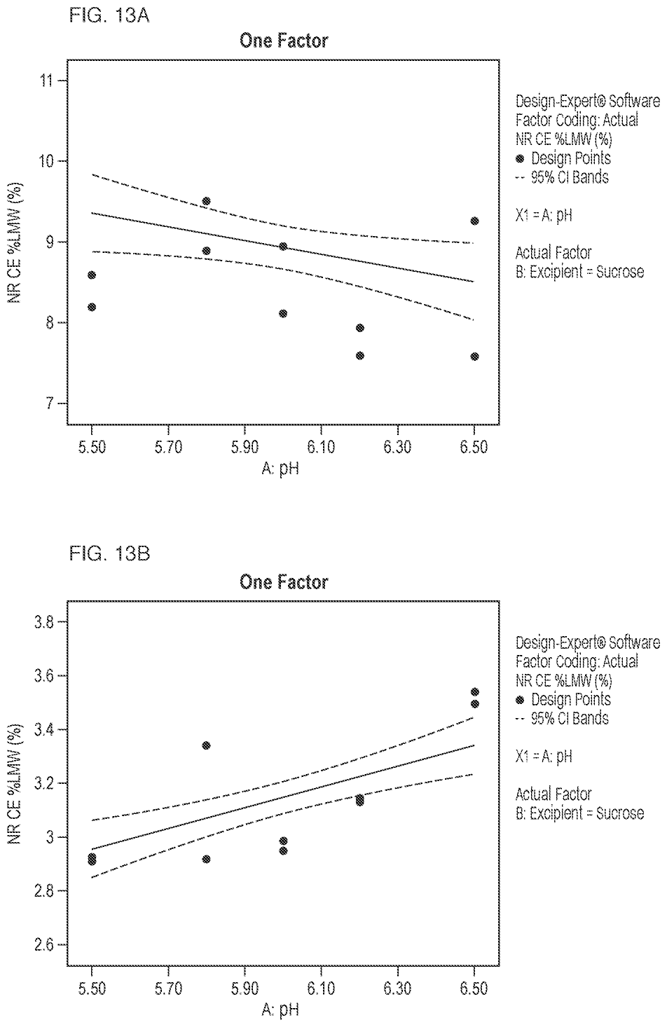

As evaludated by non-reduced CE, among the 3-week 50° C. samples, the formulations including sorbitol only or sucrose only had lower % HMW species relative to the combination excipients NaCl and sorbitol and NaCl and sucrose (FIGS. 11A and 12A). The pH level did not have a significant effect on the % main peak values.

As evaluated by reduced and non-reduced CE, there was not a significant model to fit the reduced CE data for the 3-week 2-8° C. samples, and for the non-reduced CE data the sorbitol and sucrose only formulations possessed greater % main peak species (FIG. 11B).

### Statistical Analysis

Trends were analyzed using Design Expert v9 software. A summary of the analyses is shown in Table 32. Bolded models were not included in the final optimization assessment.

TABLE 32
Summary of Analysis Models

| Condition | Assay | Response | Model | Comments |
|---|---|---|---|---|
| 3 Weeks | DLS | Z-Average | RCubic | |
| 50° C. | | PDI | RLinear | excipient not significant |
| | | Monomer Size | Linear | |
| | | % Pd | No model chosen | |
| | SEC | % HMW | RCubic | |
| | | % Main | RCubic | |
| | | % LMW | Quadratic | |
| | icIEF | % Acidic | Cubic | |
| | | % Main | Quadratic | |
| | | % Basic | RCubic | |
| | CE (R & NR) | % Purity | Quadratic | |
| | | % Impurities | Quadratic | |
| | | % Main | RLinear | pH not significant |
| | | % HMW | Linear | |
| | | % LMW | RLinear | excipient not significant |
| 3 Weeks | DLS | Z-Average | 2FI | |
| 2-8° C. | | PDI | Linear | |
| | | Monomer Size | RLinear | pH not significant |
| | | % Pd | Linear | |
| | SEC | % HMW | RQuadratic | |
| | | % Main | Linear | |
| | | % LMW | RLinear | pH not significant |
| | icIEF | % Acidic | RLinear | excipient not significant |
| | | % Main | No model chosen | no significant model |
| | | % Basic | RLinear | excipient not significant |
| | CE (R & NR) | % Purity | No model chosen | no significant model |
| | | % Impurities | No model chosen | no significant model |
| | | % Main | RLinear | pH not significant |
| | | % HMW | Linear | |
| | | % LMW | RLinear | excipient not significant |

### Excipient Selection

Performance of the formulations containing 250 mM sorbitol or 250 mM sucrose as the excipient was more desirable than the formulations containing a combination of sorbitol and NaCl or of sucrose and NaCl. Therefore, the optimal formulations for A49-F3′-TriNKET-Trastuzumab were determined to be 20 mM histidine, 250 mM sucrose or sorbitol, and 0.01% PS80, at pH 6.0.

### Example 2

### Pharmacokinetic (PK) Analysis of A49-F3′-TriNKET-Trastuzumab

This study was designed to determine the pharmacokinetic (PK) profile of A49-F3′-TriNKET-Trastuzumab when administered to cynomolgus monkeys as a 30-minute IV fusion at 1 mg/kg, 10 mg/kg, or 50 mg/kg on Day 1 and Day 15 of the study, followed by a 13- and 6-day observation period, respectively. A summary of key PK parameters following the first intravenous infusion on Day 1 of A49-F3′-TriNKET-Trastuzumab to cynomolgus male and female monkeys are presented in Table 33 and Table 34, respectively.

TABLE 33
PK parameters of A49-F3′-TriNKET-Trastuzumab in cynomolgus male monkeys
following the first intravenous infusion on Day 1.

| | | | | | AUC₀₋₁₄₄ₕₒᵤᵣₛ/ | | | | |
|---|---|---|---|---|---|---|---|---|---|
| | | Cₘₐₓ/dose | | | dose | | | | |
| Dose | Cₘₐₓ | (μg/mL) | tₘₐₓ^{a} | AUC₀₋₁₄₄ₕₒᵤᵣₛ | (μg · h/mL)/ | AUC₀₋₃₃₆ | t_{1/2} | CL | Vₛₛ |
| (mg/kg) | (μg/mL) | (mg/kg) | (hours) | (μg · h/mL) | (mg/kg) | (μg · h/mL) | (hours) | (mL/h/kg) | (mL/kg) |
| 1 | 28.6 | 28.6 | 0.25 | 1510 | 1510 | 2080 | 86.3 | 0.450 | 53.2 |
| 10 | 300 | 30 | EOI | 13800 | 1380 | 19400 | 113.6 | 0.457 | 65.8 |
| 50 | 1370 | 27.4 | 0.25 | 56100 | 1120 | 86400 | (162.1)^{b} | (0.456)^{b} | (95.2)^{b} |

Abbreviations used in the table: AUC₀₋ₜ = area under the concentration-time curve from the time of dosing to the time of the last observation;
Cₘₐₓ = maximum serum concentration observed post-dose;
CL = clearance;
tₘₐₓ = time to reach maximum concentration;
EOI = end of infusion;
t_{1/2} = halflife,
Vₛₛ = steady state volume of distribution.
^{a}Time from the end of the IV infusion;
^{b}Acceptance criteria for estimation of half-life not met-values regarded as estimates.

TABLE 34
PK parameters of A49-F3′-TriNKET-Trastuzumab in cynomolgus female
monkeys following the first intravenous infusion on Day 1.

| | | | | | AUC₀₋₁₄₄ₕₒᵤᵣₛ/ | | | | |
|---|---|---|---|---|---|---|---|---|---|
| | | Cₘₐₓ/dose | | | dose | | | | |
| Dose | Cₘₐₓ | (μg/mL) | tₘₐₓ^{a} | AUC₀₋₁₄₄ₕₒᵤᵣₛ | (μg · h/mL)/ | AUC₀₋₃₃₆ | t_{1/2} | CL | Vₛₛ |
| (mg/kg) | (μg/mL) | (mg/kg) | (hours) | (μg · h/mL) | (mg/kg) | (μg · h/mL) | (hours) | (mL/h/kg) | (mL/kg) |
| 1 | 33.1 | 33.1 | 0.25 | 1480 | 1480 | 2040 | 90.9 | 0.455 | 55.3 |
| 10 | 217 | 21.7 | 0.25 | 14100 | 1410 | 21500 | (155.3)^{b} | (0.371)^{b} | (75.8)^{b} |
| 50 | 1270 | 25.4 | 0.25 | 73200 | 1460 | 121000 | (241.6)^{b} | (0.269)^{b} | (86.1)^{b} |

Abbreviations used in the table: AUC₀₋ₜ = area under the concentration-time curve from the time of dosing to the time of the last observation;
Cₘₐₓ = maximum serum concentration observed post-dose;
CL = clearance;
tₘₐₓ = time to reach maximum concentration;
t_{1/2} = halflife,
Vₛₛ = steady state volume of distribution.
^{a}Time from the end of the IV infusion;
^{b}Acceptance criteria for estimation of half-life not met-values regarded as estimates.

A summary of key PK parameters following the second intravenous infusion on Day 15 of A49-F3′-TriNKET-Trastuzumab to cynomolgus male and female monkeys are presented in Table 35 and Table 36, respectively.

TABLE 35
PK parameters of A49-F3′-TriNKET-Trastuzumab in cynomolgus male monkeys
following the second intravenous infusion on Day 15.

| | | | | | AUC₀₋₁₄₄ₕₒᵤᵣₛ/ | | | | |
|---|---|---|---|---|---|---|---|---|---|
| | | Cₘₐₓ/dose | | | dose | | | | |
| Dose | Cₘₐₓ | (μg/mL) | tₘₐₓ^{a} | AUC₀₋₁₄₄ₕₒᵤᵣₛ | (μg · h/mL)/ | k | t_{1/2} | CL | Vₛₛ |
| (mg/kg) | (μg/mL) | (mg/kg) | (hours) | (μg · h/mL) | (mg/kg) | (hours⁻¹) | (hours) | (mL/h/kg) | (mL/kg) |
| 1 | 30.7 | 30.7 | 0.25 | 1510 | 1510 | (0.0088)^{b} | (78.5)^{b} | (0.510)^{b} | (52.2)^{b} |
| 10 | 208 | 20.8 | 0.25 | 13300 | 1330 | (0.0066)^{b} | (105.7)^{b} | (0.527)^{b} | (76.8)^{b} |
| 50 | 1260 | 25.2 | 0.5 | 70300 | 1410 | (0.0057)^{b} | (122.4)^{b} | (0.474)^{b} | (79.5)^{b} |

Abbreviations used in the table: AUC₀₋ₜ = area under the concentration-time curve from the time of dosing to the time of the last observation;
Cₘₐₓ = maximum serum concentration observed post-dose;
CL = clearance;
tₘₐₓ = time to reach maximum concentration;
t_{1/2} = halflife,
Vₛₛ = steady state volume of distribution.
^{a}Time from the end of the IV infusion;
^{b}Acceptance criteria for estimation of half-life not met-values regarded as estimates.

TABLE 36
PK parameters of A49-F3′-TriNKET-Trastuzumab in cynomolgus female
monkeys following the second intravenous infusion on Day 15.

| | | | | | AUC₀₋₁₄₄ₕₒᵤᵣₛ/ | | | | |
|---|---|---|---|---|---|---|---|---|---|
| | | Cₘₐₓ/dose | | | dose | | | | |
| Dose | Cₘₐₓ | (μg/mL) | tₘₐₓ^{a} | AUC₀₋₁₄₄ₕₒᵤᵣₛ | (μg · h/mL)/ | k | t_{1/2} | CL | Vₛₛ |
| (mg/kg) | (μg/mL) | (mg/kg) | (hours) | (μg · h/mL) | (mg/kg) | (hours⁻¹) | (hours) | (mL/h/kg) | (mL/kg) |
| 1 | 28.2 | 28.2 | 1 | 1580 | 1580 | (0.0064)^{b} | (107.6)^{b} | (0.452)^{b} | (63.3)^{b} |
| 10 | 224 | 22.4 | 0.25 | 15800 | 1580 | (0.0069)^{b} | (101.0)^{b} | (0.448)^{b} | (63.7)^{b} |
| 50 | 1590 | 31.8 | 1 | 108000 | 2160 | (0.0051)^{b} | (137.0)^{c} | (0.304)^{c} | (58.4)^{c} |

Abbreviations used in the table: AUC₀₋ₜ = area under the concentration-time curve from the time of dosing to the time of the last observation;
Cₘₐₓ = maximum serum concentration observed post-dose;
CL = clearance;
tₘₐₓ = time to reach maximum concentration;
t_{1/2} = halflife,
Vₛₛ = steady state volume of distribution.
^{a}Time from the end of the IV infusion;
^{b}Acceptance criteria for estimation of half-life not met-values regarded as estimates.

The time at which the tₘₐₓ occurred was generally 15 minutes after the end of infusion (EOI). But as shown in Tables 33 and 35, tₘₐₓ also occurred at the EOI on Day 1 in the male cynomolgus monkeys receiving 10 mg/kg and 30 minutes after the EOI on Day 15 in the male cynomolgus monkeys receiving 50 mg/kg, respectively. As shown in Tables 34 and 36, tₘₐₓ also occurred 1 hour after the EOI on Day 15 in the females receiving cynomolgus monkeys 1 mg/kg or 50 mg/kg, respectively. These data indicated that A49-F3′-TriNKET-Trastuzumab declined slowly following 30-minute IV administration with a long terminal half-life (t_{1/2}), for example, in excess of 90 hours. The data also indicated low serum clearance and that the volume of distribution was similar to the blood volume (73.4 mL/kg) and lower than the volume of total body water (693 mL/kg).

The ratio between serum A49-F3′-TriNKET-Trastuzumab maximum serum concentration (Cₘₐₓ) and area under the concentration-time curve (AUC_{0-144 hr}) for the dose levels was also evaluated. Table 37 shows Cₘₐₓ ratio and AUC ratio per dose level for A49-F3′-TriNKET-Trastuzumab.

TABLE 37
Cₘₐₓ ratio and AUC ratio per dose level for A49-F3′-TriNKET-Trastuzumab in
cynomologus male and female monkeys.

| Dose | Dose | Cₘₐₓ ratio | | | | AUC_{0-144 hours} ratio | | | |
|---|---|---|---|---|---|---|---|---|---|
| level | level | Day 1 | | Day 15 | | Day 1 | | Day 15 | |
| (mg/kg) | ratio | Males | Females | Males | Females | Males | Females | Males | Females |
| 1 | 1 | 1 | 1 | 1 | 1 | 1 | 1 | 1 | 1 |
| 10 | 10.0 | 10.5 | 6.6 | 6.8 | 7.9 | 9.1 | 9.5 | 8.8 | 10.0 |
| 50 | 50.0 | 47.9 | 38.4 | 41.0 | 56.4 | 37.2 | 49.5 | 46.6 | 68.4 |

As shown in Table 37, Cₘₐₓ and AUC_{0-144 hr} values increased approximately proportionately with increasing dose over the dose range of 1 to 50 mg/kg. The Cₘₐₓ and AUC₀₋₁₄₄ hr values of A49-F3′-TriNKET-Trastuzumab in cynomolgus female monkeys were similar to those indices of exposure in cynomolgus male monkeys. At the second injection on Day 15, the Cₘₐₓ and AUC_{0-144 hr} values of A49-F3′-TriNKET-Trastuzumab were similar to those after the first dose on Day 1 at the 1 and 10 mg/kg dose levels but were generally slightly higher at the 50 mg/kg dose level. The accumulation ratios, based on AUC_{0-144 hr} values, were slightly greater than one at the 50 mg/kg dose levels, indicating that some accumulation occurred after repeated IV infusion administrations of A49-F3′-TriNKET-Trastuzumab at this dose level.

Additionally, none of the samples from A49-F3′-TriNKET-Trastuzumab-treated animals was confirmed anti-drug antibody positive concluding that no test article-related anti-drug antibodies were observed in the study.

### Example 3

### Treatment of Locally Advanced or Metastatic Solid Tumor with A49-F3′-TriNKET-Trastuzumab

### Objectives

This clinical study is designed with two phases: dose escalation phase and efficacy followed by efficacy expansion cohorts phase. The primary objective of the dose escalation phase of the study is to assess the safety and tolerability of A49-F3′-TriNKET-Trastuzumab, and to determine the maximum tolerated dose of A49-F3′-TriNKET-Trastuzumab in patients with advanced (unresectable, recurrent or metastatic) solid tumors for whom no effective standard therapy exists or have recurrent or are intolerant of standard therapy(ies). The primary objective of the efficacy expansion cohorts phase of the study is to assess the overall response rate (ORR) according to the modified Response Evaluation Criteria in Solid Tumors version 1.1 (mRECIST 1.1) per an independent endpoint review committee (IERC).

The secondary objectives of this clinical study are:
to characterize the pharmacokinetic(s) of A49-F3′-TriNKET-Trastuzumab;
to evaluate immunogenicity of A49-F3′-TriNKET-Trastuzumab and to correlate to its exposure and clinical activity;
to assess duration of response (DOR) of A49-F3′-TriNKET-Trastuzumab per an IERC;
to assess best overall response (BOR) by an IERC;
to assess progression free survival (PFS) for A49-F3′-TriNKET-Trastuzumab per an IERC;
to assess overall survival (OS) time; and
to assess the safety of A49-F3′-TriNKET-Trastuzumab in combination therapy with pembrolizumab.

### Study Design

This study is a Phase I/II, open-label, dose escalation study with a consecutive parallel-group efficacy expansion study, designed to determine the safety, tolerability, pharmacokinetic(s) (PK), pharmacodynamic(s) (PD), and preliminary anti-tumor activity of A49-F3′-TriNKET-Trastuzumab alone and in combination with pembrolizumab. This study consists of two parts:
(1) Dose Escalation Part (Phase I) is divided into the following three phases:
   (A) accelerated titration;
   (B) “3+3” dose escalation; and
   (C) safety/pharmacokinetic(s) (PK)/pharmacodynamic(s) (PD) expansion cohorts
(2) Efficacy Expansion Cohort Part (Phase II) is divided into the following four cohorts:
   (A) Urothelial bladder cancer (UBC)
   (B) Metastatic breast cancer (MBC)
   (C) Basket solid tumors with high HER2 expression (HER2 3+)
   (D) Combination therapy with pembrolizumab.

In one exemplary embodiment, patients enrolled in the dose escalation part and in the efficacy expansion (the UBC, MBC, or Basket [HER2 3+] cohorts) part receive A49-F3′-TriNKET-Trastuzumab as monotherapy intravenously as a 1-hour infusion in 4-week treatment cycles. For treatment cycle 1, patients receive A49-F3′-TriNKET-Trastuzumab at Day 1, Day 8, and Day 15. For treatment cycle 2 and subsequent cycles, patients receive A49-F3′-TriNKET-Trastuzumab once every 2 weeks (e.g., Day 1 and Day 15) until confirmed progression, unacceptable toxicity (as described in this example under section ‘Dose-Limiting Toxicity (DLT’)), or any reason for withdrawal from the trial or investigational medicinal product (IMP) occurrence. Patients enrolled in the combination therapy with pembrolizumab cohort of the efficacy expansion cohorts part receive A49-F3′-TriNKET-Trastuzumab as a 1-hour IV infusion and pembrolizumab as a 30-minute IV infusion in 3-week treatment cycles. In one exemplary embodiment, 200 mg of pembrolizumab is administered as per its label with A49-F3′-TriNKET-Trastuzumab.

For treatment cycle 1, patients receive A49-F3′-TriNKET-Trastuzumab and pembrolizumab at Day 1, and A49-F3′-TriNKET-Trastuzumab alone at Day 8. For treatment cycle 2 and subsequent cycles, patients receive A49-F3′-TriNKET-Trastuzumab and pembrolizumab once every 3 weeks on Day 1 of every cycle until confirmed progression, unacceptable toxicity (as described in this example under section ‘Dose-Limiting Toxicity (DLT’)), or any reason for withdrawal from the trial or IMP occurrence.

Patients who experience a confirmed complete response (CR) receive treatment for a maximum of 12 months after confirmation, at the discretion of the investigator. Treatment beyond 12 months is permissible if the investigator believes that such a patient will benefit from continued treatment after discussion with the sponsor medical monitor.

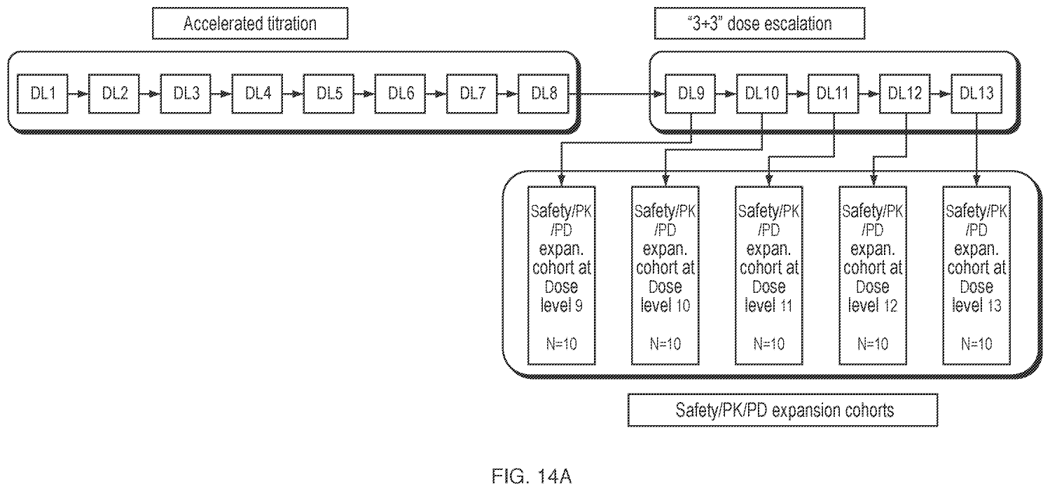

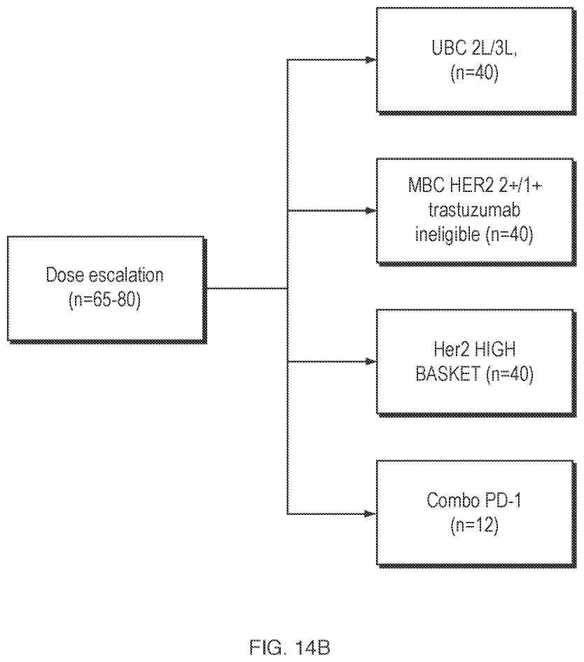

FIGS. 14A-B is a schematic diagram of the clinical trial design. FIG. 14A describes the trial design for dose escalation phase. FIG. 14B describes the trial design for efficacy expansion cohorts phase.

### Inclusion Criteria

The general inclusion criteria for patients enrolled in any of the cohorts in the clinical study of this example include:
have signed written informed consent;
are ≥18 years (include male and female patients);
have histologically or cytologically proven locally advanced or metastatic solid tumors, for which no standard therapy exists, or standard therapy has failed. Primary tumor must have documented HER2 expression by immunohistochemistry;
have ECOG performance status of 0 or 1 at study entry and an estimated life expectancy of at least 3 months;
have baseline left ventricular ejection fraction (LVEF)≥55% as measured by echocardiography (preferred) or multigated acquisition (MUGA) scan;
have adequate hematological function defined by white blood cell (WBC) count≥3×10⁹/L with absolute neutrophil count (ANC)≥1.5×10⁹/L, lymphocyte count≥0.5×10⁹/L, platelet count≥75×10⁹/L, and hemoglobin≥9 g/dL (may have been transfused);
have adequate hepatic function defined by a total bilirubin level≤1.5× the upper limit of normal (ULN), an aspartate aminotransferase (AST) level≤2.5×ULN, and an alanine aminotransferase (ALT) level≤2.5×ULN or, for patients with documented metastatic disease to the liver, AST and ALT levels≤5×ULN;
have adequate renal function defined by an estimated creatinine clearance>50 mL/min according to the Cockcroft-Gault formula; and
have effective contraception for women of child bearing potential (WOCBP) patients as defined by WHO guidelines for 1 “highly effective” method or 2 “effective” methods.

The additional inclusion criteria for patients enrolled in the accelerated titration or “3+3” dose escalation phase of the dose escalation part described in this example include:
have evidence of objective disease, but participation does not require a measurable lesion; and
have archived tumor biopsy available (≤6 months old, at least 8 slides) or fresh biopsy obtained within the screening window (at least 10 slides and 3 cores).

The additional inclusion criteria for patients enrolled in the safety/PK/PD expansion cohorts phase of the dose escalation part described in this example include:
have fresh tumor biopsy obtained during the screening window to have Formalin Fixed Paraffin Embedded (FFPE) paraffin block or enough unstained slides to perform IHC (at least 3 slides unstained) with at least 12 slides overall and with at least 3 fresh cores; and
have HER2 by IHC of at least 1+at screening.

The additional inclusion criteria for patients enrolled in the UBC expansion cohort described in this example include:
have histologically or cytologically documented locally advanced or metastatic transitional cell carcinoma of the urothelium (including renal pelvis, ureters, urinary urothelial, urethra);
have radiographic disease progression after their last line of therapy;
have received one (and no more than one) platinum-containing regimen (e.g., platinum plus another agent such as gemcitabine, methotrexate, vinblastine, doxorubicin, etc.) for inoperable locally advanced or metastatic urothelial carcinoma with radiographic progression or with recurrence within 6 months after the last administration of a platinum-containing regimen as an adjuvant, which would be considered failure of a first-line, platinum-containing regimen;
have received treatment with a checkpoint inhibitor (i.e., anti-PD-1 or anti-PD-L1), with radiographic progression (optionally have received a combination of platinum-based therapy with PD-1/PD-L1-based therapy);
have at least 1+ expression of HER2 by IHC; and
have fresh tumor biopsy obtained during the screening window to have Formalin Fixed Paraffin Embedded (FFPE) paraffin block or enough unstained slides to perform IHC (at least 3 slides unstained) with at least 12 slides overall and with at least 3 fresh cores.

The additional inclusion criteria for patients enrolled in the MBC expansion cohort described in this example include:
have histologically confirmed MBC;
have received no more than 3 prior lines of cytotoxic therapy for metastatic disease;
have received a taxane and an anthracycline unless anthracycline is contraindicated;
have a tumor scoring 1+ or 2+ by IHC, and if scoring 2+, the existence of tumor amplification of ERRB2 must have been ruled by an FDA approved method;
have progressed (radiographically) after their last line of systemic therapy; and
have fresh tumor biopsy obtained during the screening window to have Formalin Fixed Paraffin Embedded (FFPE) paraffin block or enough unstained slides to perform IHC (at least 3 slides unstained) with at least 12 slides overall and with at least 3 fresh cores.

The additional inclusion criteria for patients enrolled in the basket (HER2 3+) cohort described in this example include:
with any solid tumor except breast cancer or gastric cancer and history of ERRB2 amplification within the tumor and one of the following 1) HER2 3+ by IHC documented in the most recent biopsy within 6 months, post radiographic progression on the last line or 2) HER 3+ by IHC during the screening window;
have received at least one line of an approved or established therapy; and
have fresh tumor biopsy obtained during the screening window to have Formalin Fixed Paraffin Embedded (FFPE) paraffin block or enough unstained slides to perform IHC (at least 3 slides unstained) with at least 12 slides overall and with at least 3 fresh cores.

The additional inclusion criteria for patients enrolled in the combination therapy with pembrolizumab cohort phase of the efficacy expansion part described in this example include:
eligibility to receive pembrolizumab per its label for a malignancy of epithelial origin; and
have fresh tumor biopsy obtained during the screening window to have Formalin Fixed Paraffin Embedded (FFPE) paraffin block or enough unstained slides to perform IHC (at least 3 slides unstained) with at least 12 slides overall and with at least 3 fresh cores.

### Exclusion Criteria

The exclusion criteria for patients enrolled in the clinical study of this example include:
Concurrent treatment with a non-permitted drug including:
   Immunotherapy, immunosuppressive drugs (including chemotherapy or systemic corticosteroids except for short term treatment of allergic reactions or for the treatment of irAEs), or other experimental pharmaceutical products.
      Exceptions:
         Short term administration of systemic steroid (e.g., for allergic reactions or the management of irAEs) is allowed.
         Steroids with no or minimal systemic effect (topical, inhalation) are allowed;
   TKIs targeting HER2, or any recombinant molecule targeting HER2 or NKG2D;
   Growth factors (granulocyte colony stimulating factor or granulocyte macrophage colony stimulating factor).
      Exception:
         Erythropoietin and erythropoietin analogs may be prescribed at the Investigator's discretion;
   Bisphosphonate or denosumab treatment is not allowed.
      Exception: Bisphosphonate or denosumab is allowed unless it has been initiated more than 14 days prior to receiving the first administration of A49-F3′-TriNKET-Trastuzumab.
Previous treatment with drugs that specifically target the HER2 pathway.
   Exception: mAb or tyrosine kinase inhibitor (TKI) is acceptable providing washout period (4 weeks for mAbs or protein therapeutics and 2 weeks for a TKI);
Concurrent anticancer treatment (e.g., cytoreductive therapy, radiotherapy (with the exception of palliative bone directed radiotherapy), immune therapy, or cytokine therapy except for erythropoietin), major surgery (excluding prior diagnostic biopsy), concurrent systemic therapy with steroids or other immunosuppressive agents, or use of any investigational drug within 28 days before the start of study treatment. Short-term administration of systemic steroids (e.g., for allergic reactions or the management of irAEs) is allowed. Patients receiving bisphosphonates are eligible provided treatment was initiated at least 14 days before the first dose of A49-F3′-TriNKET-Trastuzumab;
Previous malignant disease other than the target malignancy to be investigated in this study within the last 3 years, with the exception of basal or squamous cell carcinoma of the skin or cervical carcinoma in situ;
Rapidly progressive disease;
Active or history of central nervous system (CNS) metastases;
Receipt of any organ transplantation including autologous or allogeneic stem-cell transplantation;
Significant acute or chronic infections (including historic positive test for human immunodeficiency virus (HIV), or active or latent hepatitis B or active hepatitis C tested during the screening window);
Preexisting autoimmune disease (except for patients with vitiligo) needing treatment with systemic immunosuppressive agents for more than 28 days within the last 3 years or clinically relevant immunodeficiencies (e.g., dys-gammaglobulinemia or congenital immunodeficiencies), or fever within 7 days of Day 1;
Known severe hypersensitivity reactions to mAbs (≥Grade 3 NCI-CTCAE v5.0), any history of anaphylaxis, or uncontrolled asthma (e.g., 3 or more features of partly controlled asthma);
Persisting toxicity related to prior therapy>Grade 1 NCI-CTCAE v5.0, however alopecia and sensory neuropathy≤Grade 2 is acceptable;
Pregnancy or lactation in females during the study;
Known alcohol or drug abuse;
Serious cardiac illness or medical conditions including but not limited to:
   History of New York Heart Association class III or IV heart failure or systolic dysfunction (LVEF<55%);
   High-risk uncontrolled arrhythmias ie, tachycardia with a heart rate>100/min at rest;
   Significant ventricular arrhythmia (ventricular tachycardia) or higher-grade AV-block (second degree AV-block Type 2 (Mobitz 2) or third-degree AV-block);
   Angina pectoris requiring anti-anginal medication;
   Clinically significant valvular heart disease;
   Evidence of transmural infarction on ECG;
   Poorly controlled hypertension (defined by: systolic>180 mm Hg or diastolic>100 mm Hg);
   Clinically relevant uncontrolled cardiac risk factors, clinically relevant pulmonary disease or any clinically relevant medical condition in the opinion of the Investigator that may limit participation in this study;
All other significant diseases (e.g., inflammatory bowel disease), which, in the opinion of the Investigator, might impair the patient's ability to participate;
Any psychiatric condition that would prohibit the understanding or rendering of informed consent;
Legal incapacity or limited legal capacity; or
Incapable of giving signed informed consent, which includes compliance with the requirements and restrictions listed in the informed consent form (ICF) and in this protocol.

### Dose-Limiting Toxicity (DLT)

At each cohort, safety and tolerability is accessed. Dose-limiting toxicity (DLT) is evaluated in the first 21 days for the patients enrolled in the dose escalation part and in the pembrolizumab combination cohort. A DLT is a ≥grade 3 adverse drug reaction according to the National Cancer Institute-common terminology criteria for adverse events (NCI-CTCAE) v5.0, occurring in the DLT evaluation period of the dose escalation cohorts. Adverse drug reactions may be adverse events suspected to be related to A49-F3′-TriNKET-Trastuzumab by the investigator and/or sponsor. DLT is defined as any of the following occurring within the first 21 days of treatment for the patients enrolled in the dose escalation part and in the pembrolizumab combination cohort:
Any grade 3 to 4 non-hematological toxicity except:
   i. grade 3 nausea, vomiting, and diarrhea lasting<72 hours in the absence of maximal medical therapy;
   ii. grade 4 vomiting and diarrhea lasting<72 hours in the absence of maximal medical therapy;
   iii. grade 3 fatigue<5 days;
   iv. grade 3 hypertension in the absence of maximal medical therapy.
Any of the following hematological toxicity:
   i. grade 4 neutropenia>5 days;
   ii. grade 3 thrombocytopenia with hemorrhage;
   iii. grade 4 thrombocytopenia.
And except for the following:
   i. single laboratory values out of normal range that are unlikely related to study treatment according to the Investigator, do not have any clinical correlate, and resolve to ≤Grade 1 within 7 days with adequate medical management.

The observation period for DLTs may include the first 3 weeks of investigational medicinal product treatment in the dose escalation part for all dose cohorts for all patients with data used for implementing the dose-escalation algorithm for determination of the maximum tolerated dose (MTD). Additional patients may be enrolled in the dose escalation phase and may have adverse events collected; optionally, these patients may not have a specific DLT observation period. Safety monitoring committee may adopt a conservative approach in ascribing the relevance of the treatment related-toxicity to drug. A treatment-related serious adverse event is ascribed as related to drug except where a clear relationship to the underlying disease or recognized co-morbidities is evident.

Safety is assessed through the recording, reporting, and analysis of baseline medical conditions, adverse events (AEs), physical examination findings, including vital signs and determination of left ventricular ejection fraction, electrocardiogram, and laboratory tests.

### Dosage and Administration

A49-F3′-TriNKET-Trastuzumab Dose Escalation

A dose level is assigned to each patient at trial entry. The dose levels are adapted for weight changes as needed. The decision to escalate to the next dose level is based on safety assessments after all patients of a cohort have reached Day 21 (DLT evaluation period). In certain embodiments, patients receive IV infusion of A49-F3′-TriNKET-Trastuzumab over 1 hour (e.g., 50 to 70 minutes) once every two weeks. Dosage of A49-F3′-TriNKET-Trastuzumab is calculated based on the weight of the patient as determined on the day prior to or the day of each drug administration. In an exemplary embodiment, the starting dose of A49-F3′-TriNKET-Trastuzumab is 5.2×10⁻⁵ mg/kg and the first 8 dose levels (DLs) follow an accelerated design of dose escalation and consist of single patient cohorts with escalation steps of no greater than 3.3-fold. In the event a DLT is observed, the dose escalation is switched to a “3+3” design with accrual of 5 additional patients at the dose level where the DLT is observed.

Similar to the accelerated titration phase, dose escalation will proceed with no greater than a 3.3-fold increase between dose levels in the “3+3” escalation phase. Table 38 outlines the starting dose according to body weight (mg/kg) and dose levels (DL) of the escalation scheme.

TABLE 38
Exemplary DLs (in mg/kg body weight) in “accelerated titration” and “3 + 3” dose
escalation phase.

“accelerated titration”

| DL1 | DL2 | DL3 | DL4 | DL5 | DL6 | DL7 | DL8 |
|---|---|---|---|---|---|---|---|
| 5.2 × 10⁻⁵ | 1.6 × 10⁻⁴ | 5.2 × 10⁻⁴ | 1.6 × 10⁻³ | 5.2 × 10⁻³ | 1.6 × 10^{× 2} | 5.2 × 10⁻² | 1.6 × 10⁻¹ |

“3 + 3” dose escalation

| DL9 | DL10 | DL11 | DL12 | DL13 |
|---|---|---|---|---|
| 0.52 | 1.6 | 5.2 | 10 | 20 |

In an exemplary embodiment, three patients are initially enrolled into a given dose cohort during the “3+3” phase. After the first patient is enrolled, the second is enrolled no sooner than 2 days after the second injection of A49-F3′-TriNKET-Trastuzumab to the first patient. The first administration of A49-F3′-TriNKET-Trastuzumab is given to the third patient after at least 48 hours of follow-up after the administration of A49-F3′-TriNKET-Trastuzumab to the second patient. More then 3 patients may be enrolled at a particular dose cohort (e.g., in the event of a DLT observed in the first 3 patients of a particular cohort, or after 3 patients have been enrolled at DL 7) without any pre-defined interval between treatment start, unless an infusion reaction or a cytokine release syndrome, or any Grade 3 or higher treatment related toxicity is observed during the treatment of the first 3 patients. In such an instance, the same pre-defined intervals for the first 3 patients are repeated. In the event, no DLT is observed in any of these patients, the study proceeds to enroll 3 additional patients into the next higher dose cohort. If 1 patient develops a DLT at a specific dose, an additional 3 patients are enrolled into that same dose cohort. Development of DLTs in more than 1 of 6 patients in a specific dose cohort suggests that the MTD has been exceeded, and further dose escalation is not pursued (see Dose-Limiting Toxicity (DLT) section in this example).

In an exemplary embodiment, once the safety of the DL 10 (1.6 mg/kg) is established by the safety monitoring committee, up to 10 additional patients (for a total of up to 16 patients per DL) are treated at DL9 in order to increase the safety, PK, and PD database at that DL, while accrual will carry on at DL 11 following the “3+3” rules. A similar process is applied for DLs 10 to 13. Accrual in the Safety/PK/PD Expansion Cohorts phase continue to proceed without a pre-defined observation period. Mandatory tumor biopsies are performed at screening (within 30 days before 1st investigational medicinal product) and within 1 to 7 days prior to 6th investigational medicinal product dose. The safety information is generated during the treatment of these patients and is communicated to the safety monitoring committee. The same process is implemented for the subsequent DLs of the Safety/PK/PD expansion cohorts.

Efficacy Expansion Cohorts Dosage

As mentioned previously in this example, there are 4 efficacy expansion cohorts: UBC; MBC; basket (HER2 3+) cohort with patients with HER2 high expressing solid tumors who have received at least 1 first-line treatment consisting of an established or an approved therapy; and combination therapy with pembrolizumab. The accrual in these cohorts is initiated as follows:
3 monotherapy cohorts are initiated after dose determination and schedule of A49-F3′-TriNKET-Trastuzumab in the first three cohorts (UBC, MBC, and basket (HER2 3+)
Once the safety of DL11 is established by the safety monitoring committee, the accrual of the safety run-in for the combination of A49-F3′-TriNKET-Trastuzumab with pembrolizumab is initiated. The doses of A49-F3′-TriNKET-Trastuzumab to be combined with pembrolizumab are declared safe during the “3+3” dose escalation with A49-F3′-TriNKET-Trastuzumab as a monotherapy, before testing in combination with pembrolizumab (subsequent DL during the dose escalation is cleared as safe by the safety monitoring committee).

In the first three efficacy expansion cohorts, patients receive A49-F3′-TriNKET-Trastuzumab as monotherapy. Up to 40 patients may be enrolled in each of these three expansion cohorts, with a futility analysis occurring after the first 20 patients in each cohort have been observed for at least 3 months. Mandatory tumor biopsies are performed at screening (within 30 days before 1^{st} investigational medicinal product) and within 1 to 7 days prior to the 6^{th} investigational medicinal product dose.

In the combination therapy with pembrolizumab efficacy expansion cohort, patients receive A49-F3′-TriNKET-Trastuzumab at DL 10 (as a 1-hour IV infusion) and pembrolizumab at the approved dose of 200 mg (as a 30-minute IV infusion), in 3-week treatment cycles. This safety run-in exercise follows with the same “3+3” design described before. The patients in this study meet the inclusion criteria of patients described in the ‘Inclusion Criteria’ section.

Safety during efficacy expansion cohorts (A49-F3′-TriNKET-Trastuzumab monotherapy cohorts): All safety information from participating patients is monitored on an ongoing basis by the safety monitoring committee. In an exemplary embodiment, a group of 20 patients are enrolled and followed up for 4 weeks by the safety monitoring committee. Subsequently, such safety review occurs within 4 weeks after 40 patients are treated and followed up for at least 4 weeks. Then, a similar process is implemented each time 40 patients are enrolled and followed up for at least 4 weeks.

Safety during efficacy expansion cohort (A49-F3′-TriNKET-Trastuzumab combination therapy with pembrolizumab cohort): All safety information from participating patients is monitored on an ongoing basis by the safety monitoring committee similar to as described for the “3+3” Dose Escalation part. For each patient, safety and tolerability data is reviewed by the safety monitoring committee for the 21-day DLT evaluation period, and progression to further dose administrations is dependent upon safety monitoring committee. A group of 20 patients are enrolled and followed up for 3 weeks if combination treatment is safe to proceed (as per the safety monitoring committee decision).

### Endpoints

The study is designed to evaluate primary and secondary endpoints to assess clinical benefits of A49-F3′-TriNKET-Trastuzumab, optionally in combination with pembrolizumab as treatment for patients with locally advanced or metastatic solid tumors.

Primary Endpoints and Analysis of Primary Endpoints

Occurrence of DLTs during the first three weeks of treatment is measured as a primary endpoint in the dose escalation part. Maximum tolerated dose (MTD) is determined during the dose escalation part and is defined as the highest dose level (DL) at which no more than 1 patient out of 6 patients treated experiences a DLT event. Maximum tolerated dose is determined through the individual patient data from the dose escalation part. Additionally, for the final statistical analysis, the following may be analyzed:
at each dose level, the number and proportion of patients in the DLT population who experience a DLT during the first DLT evaluation period;
at each dose level, the number and proportion of treatment-emergent adverse events experienced by patients in the DLT population during the first DLT evaluation period.

A confirmed overall response rate per mRECIST 1.1, as adjudicated by an independent endpoint review committee (IERC) is measured as a primary endpoint for the efficacy expansion cohorts. Overall response rate is defined as the best response obtained among all tumor assessment visits after start of trial treatment until documented disease progression, taking into account the following requirements for confirmation. For complete response and partial response, confirmation of the response according to mRECIST 1.1 is required. Confirmation may be evaluated at the regularly scheduled 6-week assessment interval, but no sooner than 4 weeks after the initial documentation of complete response or partial response. Confirmation of partial response may be confirmed at an assessment later than the next assessment after the initial documentation of partial response.

A best overall response of stable disease may require that an overall response of stable disease is determined at a timepoint at least 37 days after start of study treatment. The response at each scheduled tumor assessment and the best overall response is listed for each patient.

Secondary Endpoints and Analysis of Secondary Endpoints

Secondary endpoints for the study may include the following:
number, severity, and duration of treatment-emergent adverse events for all dose groups/indications according to the NCI-CTCAE v5.0;
number, severity, and duration of treatment related adverse events according to NCI-CTCAE v5.0;
duration of response according to mRECIST 1.1, per Investigator assessment;
pharmacokinetics profile;
best overall response according to mRECIST 1.1, per Investigator assessment;
progression free survival according to mRECIST 1.1, per Investigator assessment;
overall survival time;
progressive disease profile;
serum titers of anti-A49-F3′-TriNKET-Trastuzumab antibodies;
expression of HER2 on tumor tissue;
ERBB2 status (amplified/non-amplified, mutated/non-mutated);
unconfirmed response at Week 13 according to mRECIST 1.1 (for Safety/PK/PD expansion cohorts);
progression free survival time, according to mRECIST 1.1, per IERC; duration of response according to mRECIST 1.1, per IERC (for the efficacy expansion cohorts).

Efficacy parameters: The primary efficacy parameter in the expansion part is the best overall response according to mRECIST 1.1. The ORR per Investigator assessment will be determined according to mRECIST 1. The overall response rate is evaluated over the whole trial period. For a best overall response of partial response or complete response, confirmation of the response according to mRECIST 1.1 is required. The response at each scheduled tumor assessment and the best overall response is listed for each patient. The number and proportion of overall response rate (defined as complete response+partial response) is tabulated by cohort. For the HER2 high basket cohort, the number and proportion of overall response rate is tabulated for each tumor type for which there are more than 5 patients enrolled and treated for 4 weeks. Tumor types represented by fewer than 5 patients (from 1 patient to 4 patient) is represented as one sub-group. Duration of response, according to mRECIST 1.1, is calculated for each patient with a confirmed response in the expansion cohorts and is analyzed using the Kaplan-Meier method in all cohorts. Progression free survival time and overall survival time is presented in patient listings and analyzed using the Kaplan-Meier method in the full analysis set of the expansion cohorts that enrolled the full planned number of patients.

Pharmacokinetic profile: Serum concentrations of A49-F3′-TriNKET-Trastuzumab is determined by a validated method. The following PK parameters are estimated and reported:
AUC0→t: Area under the concentration-time curve from the time of dosing to the time of the last observation (calculated by linear trapezoidal summation);
AUC0→∞: Area under the curve from the time of dosing extrapolated to infinity (calculated by the linear trapezoidal summation and extrapolated to infinity using Clast/λz);
λz: Terminal elimination rate constant. The value of λz is determined from the slope of the regression line of log (concentration) vs. time with the following constraints: (i) there must be at least 3 consecutive measurable concentrations, (ii) all concentrations must be declining with time, and (iii) the correlation coefficient (r) of regression must be ≥0.95;
Cmax: Maximum serum concentration observed post-dose;
tmax: Time at which the Cmax occurs; and
t½: Elimination half-life, determined as 0.693/λz.

The PK parameters are summarized using descriptive statistics. Individual as well as mean concentration-time plots are depicted. Unresolved missing data may be imputed when the analysis integrity is affected. The conservative principle is used for data imputation.

Serum titers of anti-drug antibodies: The safety immunogenicity testing strategy is implemented and conducted in line with:
Immunogenicity Assessment of Biotechnology-Derived Therapeutic Proteins (see Guideline on Immunogenicity Assessment of Therapeutic Proteins. 18 May 2017 EMEA/CHMP/BMWP/14327/2006 Rev 1 Committee for Medicinal Products for Human Use (CHMP); European Medicines Agency);
Immunogenicity assessment of mAbs intended for in vivo clinical use (see Guideline on Immunogenicity Assessment of Monoclonal Antibodies Intended for In Vivo Clinical Use. 24 May 2012 EMA/CHMP/BMWP/86289/2010 Committee for Medicinal Products for Human Use (CHMP); European Medicines Agency);
FDA (2009, draft) Guidance for Industry: Assay Development for

Immunogenicity Testing of Therapeutic Proteins.

A qualified method that uses an acid dissociation step to detect anti-drug (ie, anti-A49-F3′-TriNKET-Trastuzumab) antibodies in the presence of excess drug in human serum is applied. Removal of drug after acid treatment is not required. ADA titers of positive samples is determined.

Biomarkers: Summary statistics for biomarkers is provided for all preplanned timepoints, separately for each DL or cohort. Changes to baseline levels are presented as applicable. Profiles over time are displayed on a per patient basis.

Safety Analyses: The extent of exposure to A49-F3′-TriNKET-Trastuzumab is characterized by duration (weeks), number of administrations, cumulative dose (mg/kg), dose intensity (mg/kg/week), relative dose intensity (actual dose given/planned dose), number of dose reductions, and number of dose delays. Safety analyses are performed on the safety population. The safety endpoints are tabulated by DL and cohort, using descriptive statistics. Safety assessments are based on review of the incidence of adverse events including adverse events of special interest, adverse drug reactions, and changes in vital signs, electrocardiograms, body weight, and laboratory values (hematology and serum chemistry). The on-treatment period is defined as the time from the first dose of study treatment to the last dose of study treatment +30 days, or the earliest date of new anticancer therapy −1 day, whichever occurs first.

Adverse Events (AEs): Adverse events are coded according to Medical Dictionary for Regulatory Activities (MedDRA). Severity of AEs is graded using the NCI-CTCAE v5.0 toxicity grading scale. Treatment-emergent adverse events (TEAEs) are those AEs with onset dates during the on-treatment period, or if the worsening of an event is during the on-treatment period. The incidence of TEAEs regardless of attribution and AEs defined as possibly related to A49-F3′-TriNKET-Trastuzumab are summarized by preferred term and system organ class and described in terms of intensity and relationship to A49-F3′-TriNKET-Trastuzumab. All premature/permanent discontinuations are summarized by primary reason for study withdrawal. Duration TEAEs is defined as the time between onset and resolution to baseline. Duration of Grade 3 and 4 is defined by the time period during which a particular TEAE reaches a Grade 3 or 4 severity during its course. Descriptive statistics are examined for indications of dose-related ADRs.

Laboratory Variables: Laboratory results are classified by Grade according to NCI-CTCAE. The worst on-trial Grades after the first trial treatment are summarized. Shifts in toxicity grading from first treatment to highest grade are displayed. Results for variables that are not part of NCI-CTCAE are presented as below, within, or above normal limits. Only patients with post-baseline laboratory values are included in these analyses.

PE (including vital signs, 12-lead electrocardiograms, and transthoracic echocardiography (TT-ECHO)/MUGA): PE data, including vital signs (body temperature, respiratory rate, heart rate, and blood pressure) and 12-lead ECG are recorded.

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

What is claimed is:

1. A pharmaceutical formulation comprising:
(a) a multi-specific binding protein comprising:
(i) a Fab that binds NKG2D;
(ii) a single-chain variable fragment (scFv) that binds HER2; and
(iii) an antibody Fc domain;
(b) histidine;
(c) a sugar or sugar alcohol; and
(d) a polysorbate,
at pH 5.5 to 6.5.

2. The pharmaceutical formulation of claim 1, wherein the concentration of histidine in the pharmaceutical formulation is 10 to 25 mM.

3. The pharmaceutical formulation of claim 2, wherein the concentration of histidine in the pharmaceutical formulation is about 20 mM.

4. The pharmaceutical formulation of any one of claims 1-3, wherein the sugar or sugar alcohol is a disaccharide.

5. The pharmaceutical formulation of claim 4, wherein the disaccharide is sucrose.

6. The pharmaceutical formulation of any one of claims 1-3, wherein the sugar or sugar alcohol is a sugar alcohol derived from a monosaccharide.

7. The pharmaceutical formulation of claim 6, wherein the sugar alcohol derived from a monosaccharide is sorbitol.

8. The pharmaceutical formulation of any one of claims 4-7, wherein the concentration of the sugar or sugar alcohol in the pharmaceutical formulation is 200 to 300 mM.

9. The pharmaceutical formulation of claim 8, wherein the concentration of the sugar or sugar alcohol in the pharmaceutical formulation is about 250 mM.

10. The pharmaceutical formulation of any one of claims 1-9, wherein the polysorbate is polysorbate 80.

11. The pharmaceutical formulation of claim 10, wherein the concentration of polysorbate 80 in the pharmaceutical formulation is 0.005% to 0.05%.

12. The pharmaceutical formulation of claim 11, wherein the concentration of polysorbate 80 in the pharmaceutical formulation is about 0.01%.

13. The pharmaceutical formulation of any one of claims 1-12, wherein the concentration of NaCl, if any, is about 10 mM or lower in the pharmaceutical formulation.

14. The pharmaceutical formulation of claim 13, wherein the concentration of NaCl, if any, is about 1 mM or lower in the pharmaceutical formulation.

15. The pharmaceutical formulation of any one of claims 1-14, wherein the pH is 5.8 to 6.2.

16. The pharmaceutical formulation of any one of claims 1-15, wherein the pH is 5.95 to 6.05.

17. The pharmaceutical formulation of any one of claims 1-16, wherein the Fab comprises a heavy chain variable domain and a light chain variable domain, and wherein
(a) the heavy chain variable domain comprises complementarity-determining region 1 (CDR1), complementarity-determining region 2 (CDR2), and complementarity-determining region 3 (CDR3) sequences represented by the amino acid sequences of SEQ ID NOs: 168, 96, and 188, respectively; and
(b) the light chain variable domain comprises CDR1, CDR2, and CDR3 sequences represented by the amino acid sequences of SEQ ID NOs: 99, 100, and 101, respectively.

18. The pharmaceutical formulation of claim 17, wherein
(a) the heavy chain variable domain comprises CDR1, CDR2, and CDR3 sequences represented by the amino acid sequences of SEQ ID NOs: 168, 96, and 169, respectively; and
(b) the light chain variable domain comprises CDR1, CDR2, and CDR3 sequences represented by the amino acid sequences of SEQ ID NOs: 99, 100, and 101, respectively.

19. The pharmaceutical formulation of any one of claims 1-18, wherein the heavy chain variable domain of the Fab comprises an amino acid sequence at least 90% identical to SEQ ID NO:94, and the light chain variable domain comprises an amino acid sequence at least 90% identical to SEQ ID NO:98.

20. The pharmaceutical formulation of any one of claims 1-19, wherein the heavy chain variable domain of the Fab comprises the amino acid sequence of SEQ ID NO:94, and the light chain variable domain comprises the amino acid sequence of SEQ ID NO:98.

21. The pharmaceutical formulation of any one of claims 1-20, wherein the scFv comprises a heavy chain variable domain and a light chain variable domain, and wherein
(a) the heavy chain variable domain comprises CDR1, CDR2, and CDR3 sequences represented by the amino acid sequences of SEQ ID NOs: 115, 116, and 117, respectively; and
(b) the light chain variable domain comprises CDR1, CDR2, and CDR3 sequences represented by the amino acid sequences of SEQ ID NOs: 119, 120, and 121, respectively.

22. The pharmaceutical formulation of claim 21, wherein the heavy chain variable domain of the scFv comprises an amino acid sequence at least 90% identical to SEQ ID NO:195, and the light chain variable domain of the scFv comprises an amino acid sequence at least 90% identical to SEQ ID NO:196.

23. The pharmaceutical formulation of claim 21 or 22, wherein the heavy chain variable domain of the scFv comprises the amino acid sequence of SEQ ID NO:195, and the light chain variable domain of the scFv comprises the amino acid sequence of SEQ ID NO:196.

24. The pharmaceutical formulation of any one of claims 21-23, wherein the light chain variable domain of the scFv is linked to the heavy chain variable domain of the scFv via a flexible linker.

25. The pharmaceutical formulation of claim 24, wherein the flexible linker comprises the amino acid sequence of SEQ ID NO:143.

26. The pharmaceutical formulation of claim 24 or 25, wherein the flexible linker consists of the amino acid sequence of SEQ ID NO:143.

27. The pharmaceutical formulation of any one of claims 21-26, wherein the light chain variable domain of the scFv is positioned to the N-terminus of the heavy chain variable domain of the scFv.

28. The pharmaceutical formulation of any one of claims 21-27, wherein the heavy chain variable domain of the scFv forms a disulfide bridge with the light chain variable domain of the scFv.

29. The pharmaceutical formulation of claim 28, wherein the disulfide bridge is formed between C44 of the heavy chain variable domain and C100 of the light chain variable domain.

30. The pharmaceutical formulation of any one of claims 21-29, wherein the scFv comprises the amino acid sequence of SEQ ID NO:139.

31. The pharmaceutical formulation of any one of claims 1-30, wherein the antibody Fc domain comprises a first antibody Fc sequence linked to the Fab and a second antibody Fc sequence linked to the scFv.

32. The pharmaceutical formulation of claim 31, wherein the first antibody Fc sequence is linked to the heavy chain portion of the Fab.

33. The pharmaceutical formulation of claim 31 or 32, wherein the scFv is linked to the second antibody Fc sequence via a hinge comprising Ala-Ser.

34. The pharmaceutical formulation of any one of claims 31-33, wherein the first and second antibody Fc sequences each comprise a hinge and a CH2 domain of a human IgG1 antibody.

35. The pharmaceutical formulation of claim 34, wherein the first and second antibody Fc sequences each comprise an amino acid sequence at least 90% identical to amino acids 234-332 of a wild-type human IgG1 antibody.

36. The pharmaceutical formulation of any one of claims 31-35, wherein the first and second antibody Fc sequences comprise different mutations promoting heterodimerization.

37. The pharmaceutical formulation of claim 36, wherein the first antibody Fc sequence is a human IgG1 Fc sequence comprising K360E and K409W substitutions.

38. The pharmaceutical formulation of claim 36 or 37, wherein the second antibody Fc sequence is a human IgG1 Fc sequence comprising Q347R, D399V, and F405T substitutions.

39. The pharmaceutical formulation of any one of claims 1-38, wherein the multi-specific binding protein comprises:
(a) a first polypeptide comprising the amino acid sequence of SEQ ID NO:141;
(b) a second polypeptide comprising the amino acid sequence of SEQ ID NO:140; and
(c) a third polypeptide comprising the amino acid sequence of SEQ ID NO:142.

40. The pharmaceutical formulation of any one of claims 1-39, wherein the concentration of the multi-specific binding protein in the pharmaceutical formulation is about 10 to about 20 mg/mL.

41. The pharmaceutical formulation of any one of claims 1-40, wherein more than 94% of the multi-specific binding protein has native conformation, as determined by size-exclusion chromatography, after incubation at 50° C. for 3 weeks.

42. The pharmaceutical formulation of any one of claims 1-41, wherein less than 4% of the multi-specific binding protein form a high molecular weight complex, as determined by size-exclusion chromatography, after incubation at 50° C. for 3 weeks.

43. The pharmaceutical formulation of any one of claims 1-42 for use in treating cancer.

44. The pharmaceutical formulation for use of claim 43, wherein the pharmaceutical formulation is diluted with 0.9% NaCl solution prior to the use.

45. A method for treating cancer, the method comprising administering to a subject in need thereof a multi-specific binding protein in an initial four-week treatment cycle on Day 1, Day 8, and Day 15, wherein the multi-specific binding protein comprises:
(a) a Fab that binds NKG2D;
(b) an scFv that binds HER2; and
(c) an antibody Fc domain.

46. The method of claim 45, further comprising administering to the subject, after the initial treatment cycle, the multi-specific binding protein in one or more subsequent four-week treatment cycles, wherein the multi-specific binding protein is administered on Day 1 and Day 15 in each subsequent treatment cycle.

47. The method of claim 45 or 46, wherein each of the doses comprises the multi-specific binding protein at an amount selected from the group consisting of 5.2×10⁻⁵ mg/kg, 1.6×10⁻⁴ mg/kg, 5.2×10⁻⁴ mg/kg, 1.6×10⁻³ mg/kg, 5.2×10⁻³ mg/kg, 1.6×10⁻² mg/kg, 5.2×10⁻² mg/kg, 1.6×10⁻¹ mg/kg, 0.52 mg/kg, 1.0 mg/kg, 1.6 mg/kg, 5.2 mg/kg, 10 mg/kg, 20 mg/kg, and 50 mg/kg.

48. The method of any one of claims 45-47, wherein the multi-specific binding protein is administered by intravenous infusion.

49. The method of any one of claims 45-48, wherein the multi-specific binding protein is used as a monotherapy.

50. The method of any one of claims 45-48, further comprising administering to the subject an anti-PD-1 antibody.

51. The method of claim 50, wherein the anti-PD-1 antibody is pembrolizumab.

52. The method of claim 51, wherein 200 mg of pembrolizumab is administered on Day 1 of the initial treatment cycle.

53. The method of claim 51 or 52, wherein if the subject receives one or more subsequent treatment cycles, 200 mg of pembrolizumab is administered once every three weeks in the subsequent treatment cycles.

54. The method of any one of claims 45-53, wherein the cancer is HER2-positive as determined by immunohistochemistry.

55. The method of any one of claims 45-53, wherein the HER2 level in the cancer is scored at least 1+ as determined by immunohistochemistry.

56. The method of claim 54 or 55, wherein the HER2 level in the cancer is scored 2+ or 3+.

57. The method of claim 54 or 55, wherein the HER2 level in the cancer is scored 3+.

58. The method of any one of claims 45-57, wherein the cancer has amplification of the ERBB2 gene.

59. The method of claim 58, wherein ERBB2 gene amplification is determined by fluorescent in situ hybridization.

60. The method of claim 58, wherein ERBB2 gene amplification is determined by DNA sequencing.

61. The method of any one of claims 45-60, wherein the cancer is a solid tumor.

62. The method of claim 61, wherein the cancer is a locally advanced or metastatic solid tumor.

63. The method of claim 62, wherein the cancer is urothelial bladder cancer or metastatic breast cancer.

64. The method of claim 61 or 62, wherein the cancer is selected from the group consisting of gastric cancer, colorectal cancer, non-small cell lung cancer (NSCLC), head and neck cancer, biliary tract cancer, glioblastoma, sarcoma, uterine cancer, cervical cancer, ovarian cancer, esophageal cancer, squamous carcinoma of the skin, prostate cancer, carcinoma of the salivary gland, breast cancer, pancreatic cancer, and gallbladder cancer.

65. The method of any one of claims 45-64, wherein the Fab comprises a heavy chain variable domain and a light chain variable domain, and wherein
(a) the heavy chain variable domain comprises CDR1, CDR2, and CDR3 sequences represented by the amino acid sequences of SEQ ID NOs: 168, 96, and 188, respectively; and
(b) the light chain variable domain comprises CDR1, CDR2, and CDR3 sequences represented by the amino acid sequences of SEQ ID NOs: 99, 100, and 101, respectively.

66. The method of claim 65, wherein
(a) the heavy chain variable domain comprises CDR1, CDR2, and CDR3 sequences represented by the amino acid sequences of SEQ ID NOs: 168, 96, and 169, respectively; and
(b) the light chain variable domain comprises CDR1, CDR2, and CDR3 sequences represented by the amino acid sequences of SEQ ID NOs: 99, 100, and 101, respectively.

67. The method of any one of claims 45-66, wherein the heavy chain variable domain of the Fab comprises an amino acid sequence at least 90% identical to SEQ ID NO:94, and the light chain variable domain comprises an amino acid sequence at least 90% identical to SEQ ID NO:98.

68. The method of any one of claims 45-67, wherein the heavy chain variable domain of the Fab comprises the amino acid sequence of SEQ ID NO:94, and the light chain variable domain comprises the amino acid sequence of SEQ ID NO:98.

69. The method of any one of claims 45-68, wherein the scFv comprises a heavy chain variable domain and a light chain variable domain, and wherein
(a) the heavy chain variable domain comprises CDR1, CDR2, and CDR3 sequences represented by the amino acid sequences of SEQ ID NOs: 115, 116, and 117, respectively; and
(b) the light chain variable domain comprises CDR1, CDR2, and CDR3 sequences represented by the amino acid sequences of SEQ ID NOs: 119, 120, and 121, respectively.

70. The method of claim 69, wherein the heavy chain variable domain of the scFv comprises an amino acid sequence at least 90% identical to SEQ ID NO:195, and the light chain variable domain of the scFv comprises an amino acid sequence at least 90% identical to SEQ ID NO:196.

71. The method of claim 69 or 70, wherein the heavy chain variable domain of the scFv comprises the amino acid sequence of SEQ ID NO:195, and the light chain variable domain of the scFv comprises the amino acid sequence of SEQ ID NO:196.

72. The method of any one of claims 69-71, wherein the light chain variable domain of the scFv is linked to the heavy chain variable domain of the scFv via a flexible linker.

73. The method of claim 72, wherein the flexible linker comprises the amino acid sequence of SEQ ID NO:143.

74. The method of claim 72 or 73, wherein the flexible linker consists of the amino acid sequence of SEQ ID NO:143.

75. The method of any one of claims 69-74, wherein the light chain variable domain of the scFv is positioned to the N-terminus of the heavy chain variable domain of the scFv.

76. The method of any one of claims 69-75, wherein the heavy chain variable domain of the scFv forms a disulfide bridge with the light chain variable domain of the scFv.

77. The method of claim 76, wherein the disulfide bridge is formed between C44 of the heavy chain variable domain and C100 of the light chain variable domain.

78. The method of any one of claims 69-77, wherein the scFv comprises the amino acid sequence of SEQ ID NO:139.

79. The method of any one of claims 45-78, wherein the antibody Fc domain comprises a first antibody Fc sequence linked to the Fab and a second antibody Fc sequence linked to the scFv.

80. The method of claim 79, wherein the first antibody Fc sequence is linked to the heavy chain portion of the Fab.

81. The method of claim 79 or 80, wherein the scFv is linked to the second antibody Fc sequence via a hinge comprising Ala-Ser.

82. The method of any one of claims 79-81, wherein the first and second antibody Fc sequences each comprise a hinge and a CH2 domain of a human IgG1 antibody.

83. The method of claim 82, wherein the first and second antibody Fc sequences each comprise an amino acid sequence at least 90% identical to amino acids 234-332 of a wild-type human IgG1 antibody.

84. The method of any one of claims 79-83, wherein the first and second antibody Fc sequences comprise different mutations promoting heterodimerization.

85. The method of claim 84, wherein the first antibody Fc sequence is a human IgG1 Fc sequence comprising K360E and K409W substitutions.

86. The method of claim 84 or 85, wherein the second antibody Fc sequence is a human IgG1 Fc sequence comprising Q347R, D399V, and F405T substitutions.

87. The method of any one of claims 45-86, wherein the multi-specific binding protein comprises:
(a) a first polypeptide comprising the amino acid sequence of SEQ ID NO:141;
(b) a second polypeptide comprising the amino acid sequence of SEQ ID NO:140; and
(c) a third polypeptide comprising the amino acid sequence of SEQ ID NO:142.

88. The method of any one of claims 45-87, wherein the multi-specific binding protein is in a pharmaceutical formulation of pH 5.5 to 6.5 comprising histidine, a sugar or sugar alcohol, and a polysorbate.

89. The method of claim 88, wherein the concentration of histidine in the pharmaceutical formulation is 10 to 25 mM.

90. The method of claim 89, wherein the concentration of histidine in the pharmaceutical formulation is about 20 mM.

91. The method of any one of claims 88-90, wherein the sugar or sugar alcohol in the pharmaceutical formulation is a disaccharide.

92. The method of claim 91, wherein the disaccharide is sucrose.

93. The method of any one of claims 88-92, wherein the sugar or sugar alcohol in the pharmaceutical formulation is a sugar alcohol derived from a monosaccharide.

94. The method of claim 93, wherein the sugar alcohol derived from a monosaccharide is sorbitol.

95. The method of any one of claims 88-94, wherein the concentration of the sugar or sugar alcohol in the pharmaceutical formulation is 200 to 300 mM.

96. The method of claim 95, wherein the concentration of the sugar or sugar alcohol in the pharmaceutical formulation is about 250 mM.

97. The method of any one of claims 88-96, wherein the polysorbate in the pharmaceutical formulation is polysorbate 80.

98. The method of claim 97, wherein the concentration of polysorbate 80 in the pharmaceutical formulation is 0.005% to 0.05%.

99. The method of claim 98, wherein the concentration of polysorbate 80 in the pharmaceutical formulation is about 0.01%.

100. The method of any one of claims 88-99, wherein the concentration of NaCl, if any, is about 10 mM or lower in the pharmaceutical formulation.

101. The method of claim 100, wherein the concentration of NaCl, if any, is about 1 mM or lower in the pharmaceutical formulation.

102. The method of any one of claims 88-101, wherein the pH of the pharmaceutical formulation is 5.8 to 6.2.

103. The method of any one of claims 88-102, wherein the pH of the pharmaceutical formulation is 5.95 to 6.05.

104. The method of any one of claims 88-103, wherein the concentration of the multi-specific binding protein in the pharmaceutical formulation is about 10 to about 20 mg/mL.

105. The method of any one of claims 88-104, wherein more than 94% of the multi-specific binding protein in the pharmaceutical formulation has native conformation, as determined by size-exclusion chromatography, after incubation at 50° C. for 3 weeks.

106. The method of any one of claims 88-105, wherein less than 4% of the multi-specific binding protein in the pharmaceutical formulation form a high molecular weight complex, as determined by size-exclusion chromatography, after incubation at 50° C. for 3 weeks.

107. The method of any one of claims 88-106, wherein the pharmaceutical formulation is diluted with 0.9% NaCl solution prior to administering to the subject in need thereof.


SEQUENCE LISTING (205 sequences)

SEQ ID NO: 1 - protein, 117 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 2 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 3 - protein, 9 aa, Artificial Sequence
  Description of Artificial Sequence Synthetic peptide
GSFSGYYWS

SEQ ID NO: 4 - protein, 16 aa, Artificial Sequence
  Description of Artificial Sequence Synthetic peptide
EIDHSGSTNYNPSLKS

SEQ ID NO: 5 - protein, 11 aa, Artificial Sequence
  Description of Artificial Sequence Synthetic peptide
ARARGPWSFDP

SEQ ID NO: 6 - protein, 117 aa, Artificial Sequence; identical to SEQ ID NO: 1
  Description of Artificial Sequence Synthetic polypeptide

SEQ ID NO: 7 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGGGTKVEIK

SEQ ID NO: 8 - protein, 117 aa, Artificial Sequence; identical to SEQ ID NO: 1
  Description of Artificial Sequence Synthetic polypeptide

SEQ ID NO: 9 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFYTFGGGTKVEIK

SEQ ID NO: 10 - protein, 117 aa, Artificial Sequence; identical to SEQ ID NO: 1
  Description of Artificial Sequence Synthetic polypeptide

SEQ ID NO: 11 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQSNSYYTFGGGTKVEIK

SEQ ID NO: 12 - protein, 117 aa, Artificial Sequence; identical to SEQ ID NO: 1
  Description of Artificial Sequence Synthetic polypeptide

SEQ ID NO: 13 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 14 - protein, 117 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWGFDPWGQGTLVTVSS

SEQ ID NO: 15 - protein, 107 aa, Artificial Sequence
  Description of Artificial Sequence Synthetic polypeptide
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 16 - protein, 117 aa, Artificial Sequence; identical to SEQ ID NO: 1
  Description of Artificial Sequence Synthetic polypeptide

SEQ ID NO: 17 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 18 - protein, 117 aa, Artificial Sequence; identical to SEQ ID NO: 1
  Description of Artificial Sequence Synthetic polypeptide

SEQ ID NO: 19 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTDFTLTISSLQPDDFATYYCQQSKEVPWTFGQGTKVEIK

SEQ ID NO: 20 - protein, 117 aa, Artificial Sequence; identical to SEQ ID NO: 1
  Description of Artificial Sequence Synthetic polypeptide

SEQ ID NO: 21 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSFPTFGGGTKVEIK

SEQ ID NO: 22 - protein, 117 aa, Artificial Sequence; identical to SEQ ID NO: 1
  Description of Artificial Sequence Synthetic polypeptide

SEQ ID NO: 23 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYDIYPTFGGGTKVEIK

SEQ ID NO: 24 - protein, 117 aa, Artificial Sequence; identical to SEQ ID NO: 1
  Description of Artificial Sequence Synthetic polypeptide

SEQ ID NO: 25 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSYPTFGGGTKVEIK

SEQ ID NO: 26 - protein, 117 aa, Artificial Sequence; identical to SEQ ID NO: 1
  Description of Artificial Sequence Synthetic polypeptide

SEQ ID NO: 27 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPTFGGGTKVEIK

SEQ ID NO: 28 - protein, 117 aa, Artificial Sequence; identical to SEQ ID NO: 1
  Description of Artificial Sequence Synthetic polypeptide

SEQ ID NO: 29 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSFPTFGGGTKVEIK

SEQ ID NO: 30 - protein, 117 aa, Artificial Sequence; identical to SEQ ID NO: 1
  Description of Artificial Sequence Synthetic polypeptide

SEQ ID NO: 31 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYSSFSTFGGGTKVEIK

SEQ ID NO: 32 - protein, 117 aa, Artificial Sequence; identical to SEQ ID NO: 1
  Description of Artificial Sequence Synthetic polypeptide

SEQ ID NO: 33 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYESYSTFGGGTKVEIK

SEQ ID NO: 34 - protein, 117 aa, Artificial Sequence; identical to SEQ ID NO: 1
  Description of Artificial Sequence Synthetic polypeptide

SEQ ID NO: 35 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSFITFGGGTKVEIK

SEQ ID NO: 36 - protein, 117 aa, Artificial Sequence; identical to SEQ ID NO: 1
  Description of Artificial Sequence Synthetic polypeptide

SEQ ID NO: 37 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSYPTFGGGTKVEIK

SEQ ID NO: 38 - protein, 117 aa, Artificial Sequence; identical to SEQ ID NO: 1
  Description of Artificial Sequence Synthetic polypeptide

SEQ ID NO: 39 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFPTFGGGTKVEIK

SEQ ID NO: 40 - protein, 117 aa, Artificial Sequence; identical to SEQ ID NO: 1
  Description of Artificial Sequence Synthetic polypeptide

SEQ ID NO: 41 - protein, 107 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYELYSYTFGGGTKVEIK

SEQ ID NO: 42 - protein, 117 aa, Artificial Sequence; identical to SEQ ID NO: 1
  Description of Artificial Sequence Synthetic polypeptide

SEQ ID NO: 43 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDTFITFGGGTKVEIK

SEQ ID NO: 44 - protein, 125 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDSSIRHAYYYYGMDVWGQGTTVTVSS

SEQ ID NO: 45 - protein, 9 aa, Artificial Sequence
  Description of Artificial Sequence Synthetic peptide
GTFSSYAIS

SEQ ID NO: 46 - protein, 17 aa, Artificial Sequence
  Description of Artificial Sequence Synthetic peptide
GIIPIFGTANYAQKFQG

SEQ ID NO: 47 - protein, 18 aa, Artificial Sequence
  Description of Artificial Sequence Synthetic peptide
ARGDSSIRHAYYYYGMDV

SEQ ID NO: 48 - protein, 113 aa, Artificial Sequence
  Description of Artificial Sequence Synthetic polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGGGTKVEIK

SEQ ID NO: 49 - protein, 17 aa, Artificial Sequence
  Description of Artificial Sequence Synthetic peptide
KSSQSVLYSSNNKNYLA

SEQ ID NO: 50 - protein, 7 aa, Artificial Sequence
  Description of Artificial Sequence Synthetic peptide
WASTRES

SEQ ID NO: 51 - protein, 9 aa, Artificial Sequence
  Description of Artificial Sequence Synthetic peptide
QQYYSTPIT

SEQ ID NO: 52 - protein, 121 aa, Artificial Sequence
  Description of Artificial Sequence Synthetic polypeptide
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSDRFHPYFDYWGQGTLVTVSS

SEQ ID NO: 53 - protein, 11 aa, Artificial Sequence
  Description of Artificial Sequence Synthetic peptide
GSISSSSYYWG

SEQ ID NO: 54 - protein, 16 aa, Artificial Sequence
  Description of Artificial Sequence Synthetic peptide
SIYYSGSTYYNPSLKS

SEQ ID NO: 55 - protein, 13 aa, Artificial Sequence
  Description of Artificial Sequence Synthetic peptide
ARGSDRFHPYFDY

SEQ ID NO: 56 - protein, 107 aa, Artificial Sequence
  Description of Artificial Sequence Synthetic polypeptide
EIVLTQSPATLSLSPGERATLSCRASQSVSRYLAWYQQKPGQAPRLLIYDASNRATGIPARFSGSGSGTDFTLTISSLEPEDFAVYYCQQFDTWPPTFGGGTKVEIK

SEQ ID NO: 57 - protein, 11 aa, Artificial Sequence
  Description of Artificial Sequence Synthetic peptide
RASQSVSRYLA

SEQ ID NO: 58 - protein, 7 aa, Artificial Sequence
  Description of Artificial Sequence Synthetic peptide
DASNRAT

SEQ ID NO: 59 - protein, 9 aa, Artificial Sequence
  Description of Artificial Sequence Synthetic peptide
QQFDTWPPT

SEQ ID NO: 60 - protein, 117 aa, Artificial Sequence; identical to SEQ ID NO: 1
  Description of Artificial Sequence Synthetic polypeptide

SEQ ID NO: 61 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCEQYDSYPTFGGGTKVEIK

SEQ ID NO: 62 - protein, 126 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGRKASGSFYYYYGMDVWGQGTTVTVSS

SEQ ID NO: 63 - protein, 9 aa, Artificial Sequence
  Description of Artificial Sequence Synthetic peptide
GTFSSYAIS

SEQ ID NO: 64 - protein, 17 aa, Artificial Sequence
  Description of Artificial Sequence Synthetic peptide
GIIPIFGTANYAQKFQG

SEQ ID NO: 65 - protein, 19 aa, Artificial Sequence
  Description of Artificial Sequence Synthetic peptide
ARRGRKASGSFYYYYGMDV

SEQ ID NO: 66 - protein, 113 aa, Artificial Sequence [sequence omitted]

SEQ ID NO: 67 - protein, 17 aa, Artificial Sequence
  Description of Artificial Sequence Synthetic peptide
ESSQSLLNSGNQKNYLT

SEQ ID NO: 68 - protein, 7 aa, Artificial Sequence
  Description of Artificial Sequence Synthetic peptide
WASTRES

SEQ ID NO: 69 - protein, 9 aa, Artificial Sequence
  Description of Artificial Sequence Synthetic peptide
QNDYSYPYT

SEQ ID NO: 70 - protein, 126 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGAPNYGDTTHDYYYMDVWGKGTTVTVSS

SEQ ID NO: 71 - protein, 9 aa, Artificial Sequence
  Description of Artificial Sequence Synthetic peptide
YTFTSYYMH

SEQ ID NO: 72 - protein, 17 aa, Artificial Sequence
  Description of Artificial Sequence Synthetic peptide
IINPSGGSTSYAQKFQG

SEQ ID NO: 73 - protein, 19 aa, Artificial Sequence
  Description of Artificial Sequence Synthetic peptide
ARGAPNYGDTTHDYYYMDV

SEQ ID NO: 74 - protein, 107 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYDDWPFTFGGGTKVEIK

SEQ ID NO: 75 - protein, 11 aa, Artificial Sequence
  Description of Artificial Sequence Synthetic peptide
RASQSVSSNLA

SEQ ID NO: 76 - protein, 7 aa, Artificial Sequence
  Description of Artificial Sequence Synthetic peptide
GASTRAT

SEQ ID NO: 77 - protein, 9 aa, Artificial Sequence
  Description of Artificial Sequence Synthetic peptide
QQYDDWPFT

SEQ ID NO: 78 - protein, 124 aa, Artificial Sequence
  Description of Artificial Sequence Synthetic polypeptide
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDTGEYYDTDDHGMDVWGQGTTVTVSS

SEQ ID NO: 79 - protein, 9 aa, Artificial Sequence
  Description of Artificial Sequence Synthetic peptide
YTFTGYYMH

SEQ ID NO: 80 - protein, 17 aa, Artificial Sequence
  Description of Artificial Sequence Synthetic peptide
WINPNSGGTNYAQKFQG

SEQ ID NO: 81 - protein, 17 aa, Artificial Sequence
  Description of Artificial Sequence Synthetic peptide
ARDTGEYYDTDDHGMDV

SEQ ID NO: 82 - protein, 107 aa, Artificial Sequence
  Description of Artificial Sequence Synthetic polypeptide
EIVLTQSPGTLSLSPGERATLSCRASQSVSSNLAWYQQKPGQAPRLLIYGASTRATGIPARFSGSGSGTEFTLTISSLQSEDFAVYYCQQDDYWPPTFGGGTKVEIK

SEQ ID NO: 83 - protein, 50 aa, Artificial Sequence
  Description of Artificial Sequence Synthetic polypeptide
GGSGGGGSGGGGSGGGGSGGGGSGGGGSGGGGSGGGGSGGGGSGGGGSGG

SEQ ID NO: 84 - protein, 50 aa, Artificial Sequence
  Description of Artificial Sequence Synthetic polypeptide
GGGGSGGGGSGGGGSGGGGSGGGGSGGGGSGGGGSGGGGSGGGGSGGGGS

SEQ ID NO: 85 - protein, 9 aa, Artificial Sequence
  Description of Artificial Sequence Synthetic peptide
QQDDYWPPT

SEQ ID NO: 86 - protein, 121 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSS

SEQ ID NO: 87 - protein, 9 aa, Artificial Sequence
  Description of Artificial Sequence Synthetic peptide
FTFSSYAMS

SEQ ID NO: 88 - protein, 17 aa, Artificial Sequence
  Description of Artificial Sequence Synthetic peptide
AISGSGGSTYYADSVKG

SEQ ID NO: 89 - protein, 14 aa, Artificial Sequence
  Description of Artificial Sequence Synthetic peptide
AKDGGYYDSGAGDY

SEQ ID NO: 90 - protein, 107 aa, Artificial Sequence
  Description of Artificial Sequence Synthetic polypeptide
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIK

SEQ ID NO: 91 - protein, 11 aa, Artificial Sequence
  Description of Artificial Sequence Synthetic peptide
RASQGIDSWLA

SEQ ID NO: 92 - protein, 7 aa, Artificial Sequence
  Description of Artificial Sequence Synthetic peptide
AASSLQS

SEQ ID NO: 93 - protein, 9 aa, Artificial Sequence
  Description of Artificial Sequence Synthetic peptide
QQGVSYPRT

SEQ ID NO: 94 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 95 - protein, 9 aa, Artificial Sequence
  Description of Artificial Sequence Synthetic peptide
FTFSSYSMN

SEQ ID NO: 96 - protein, 17 aa, Artificial Sequence
  Description of Artificial Sequence Synthetic peptide
SISSSSSYIYYADSVKG

SEQ ID NO: 97 - protein, 15 aa, Artificial Sequence
  Description of Artificial Sequence Synthetic peptide
ARGAPMGAAAGWFDP

SEQ ID NO: 98 - protein, 107 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 99 - protein, 11 aa, Artificial Sequence
  Description of Artificial Sequence Synthetic peptide
RASQGISSWLA

SEQ ID NO: 100 - protein, 7 aa, Artificial Sequence
  Description of Artificial Sequence Synthetic peptide
AASSLQS

SEQ ID NO: 101 - protein, 9 aa, Artificial Sequence
  Description of Artificial Sequence Synthetic peptide
QQGVSFPRT

SEQ ID NO: 102 - protein, 125 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREGAGFAYGMDYYYMDVWGKGTTVTVSS

SEQ ID NO: 103 - protein, 40 aa, Artificial Sequence
  Description of Artificial Sequence Synthetic polypeptide
GGGSGGGSGGGSGGGSGGGSGGGSGGGSGGGSGGGSGGGS

SEQ ID NO: 104 - protein, 40 aa, Artificial Sequence
  Description of Artificial Sequence Synthetic polypeptide
GGSGGGSGGGSGGGSGGGSGGGSGGGSGGGSGGGSGGGSG

SEQ ID NO: 105 - protein, 18 aa, Artificial Sequence
  Description of Artificial Sequence Synthetic peptide
AREGAGFAYGMDYYYMDV

SEQ ID NO: 106 - protein, 107 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSDNWPFTFGGGTKVEIK

SEQ ID NO: 107 - protein, 11 aa, Artificial Sequence
  Description of Artificial Sequence Synthetic peptide
RASQSVSSYLA

SEQ ID NO: 108 - protein, 7 aa, Artificial Sequence
  Description of Artificial Sequence Synthetic peptide
DASNRAT

SEQ ID NO: 109 - protein, 9 aa, Artificial Sequence
  Description of Artificial Sequence Synthetic peptide
QQSDNWPFT

SEQ ID NO: 110 - protein, 121 aa, Artificial Sequence
  Description of Artificial Sequence Synthetic polypeptide
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 111 - protein, 110 aa, Artificial Sequence
  Description of Artificial Sequence Synthetic polypeptide
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 112 - protein, 115 aa, Artificial Sequence
  Description of Artificial Sequence Synthetic polypeptide
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 113 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 114 - protein, 120 aa, Artificial Sequence [sequence omitted]

SEQ ID NO: 115 - protein, 7 aa, Artificial Sequence
  Description of Artificial Sequence Synthetic peptide
GFNIKDT

SEQ ID NO: 116 - protein, 6 aa, Artificial Sequence
  Description of Artificial Sequence Synthetic peptide
YPTNGY

SEQ ID NO: 117 - protein, 11 aa, Artificial Sequence
  Description of Artificial Sequence Synthetic peptide
WGGDGFYAMDY

SEQ ID NO: 118 - protein, 107 aa, Artificial Sequence [sequence omitted]

SEQ ID NO: 119 - protein, 8 aa, Artificial Sequence
  Description of Artificial Sequence Synthetic peptide
QDVNTAVA

SEQ ID NO: 120 - protein, 7 aa, Artificial Sequence
  Description of Artificial Sequence Synthetic peptide
SASFLYS

SEQ ID NO: 121 - protein, 9 aa, Artificial Sequence
  Description of Artificial Sequence Synthetic peptide
QQHYTTPPT

SEQ ID NO: 122 - protein, 120 aa, Artificial Sequence
  Description of Artificial Sequence Synthetic polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSA

SEQ ID NO: 123 - protein, 7 aa, Artificial Sequence
  Description of Artificial Sequence Synthetic peptide
GFTFTDY

SEQ ID NO: 124 - protein, 6 aa, Artificial Sequence
  Description of Artificial Sequence Synthetic peptide
NPNSGG

SEQ ID NO: 125 - protein, 10 aa, Artificial Sequence
  Description of Artificial Sequence Synthetic peptide
NLGPSFYFDY

SEQ ID NO: 126 - protein, 108 aa, Artificial Sequence
  Description of Artificial Sequence Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKR

SEQ ID NO: 127 - protein, 8 aa, Artificial Sequence
  Description of Artificial Sequence Synthetic peptide
QDVSIGVA

SEQ ID NO: 128 - protein, 7 aa, Artificial Sequence
  Description of Artificial Sequence Synthetic peptide
SASYRYT

SEQ ID NO: 129 - protein, 9 aa, Artificial Sequence
  Description of Artificial Sequence Synthetic peptide
QQYYIYPYT

SEQ ID NO: 130 - protein, 120 aa, Artificial Sequence
  Description of Artificial Sequence Synthetic polypeptide
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSS

SEQ ID NO: 131 - protein, 7 aa, Artificial Sequence
  Description of Artificial Sequence Synthetic peptide
GFNIKDT

SEQ ID NO: 132 - protein, 6 aa, Artificial Sequence
  Description of Artificial Sequence Synthetic peptide
YPTNGY

SEQ ID NO: 133 - protein, 11 aa, Artificial Sequence
  Description of Artificial Sequence Synthetic peptide
WGGDGFYAMDY

SEQ ID NO: 134 - protein, 107 aa, Artificial Sequence
  Description of Artificial Sequence Synthetic polypeptide
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIK

SEQ ID NO: 135 - protein, 8 aa, Artificial Sequence
  Description of Artificial Sequence Synthetic peptide
QDVNTAVA

SEQ ID NO: 136 - protein, 7 aa, Artificial Sequence
  Description of Artificial Sequence Synthetic peptide
SASFRYT

SEQ ID NO: 137 - protein, 9 aa, Artificial Sequence
  Description of Artificial Sequence Synthetic peptide
QQHYTTPPT

SEQ ID NO: 138 - protein, 1255 aa, Artificial Sequence [sequence omitted]

SEQ ID NO: 139 - protein, 247 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSS

SEQ ID NO: 140 - protein, 475 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 141 - protein, 451 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 142 - protein, 210 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIKRTVAAPSPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 20 aa, Artificial Sequence
  Description of Artificial Sequence Synthetic peptide
GGGGSGGGGSGGGGSGGGGS

SEQ ID NO: 144 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSS

SEQ ID NO: 145 - protein, 451 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 146 - protein, 475 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 147 - protein, 451 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 148 - protein, 450 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 149 - protein, 214 aa, Artificial Sequence
  Description of Artificial Sequence Synthetic polypeptide
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 15 aa, Artificial Sequence
  Description of Artificial Sequence Synthetic peptide
GGGGSGGGGSGGGGS

SEQ ID NO: 151 - protein, 80 aa, Artificial Sequence
  Description of Artificial Sequence Synthetic polypeptide
  MISC_FEATURE (1)..(80): This sequence may encompass 1-20 “Gly Gly Gly Ser” repeating units
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 152 - protein, 100 aa, Artificial Sequence
  Description of Artificial Sequence Synthetic polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 153 - protein, 80 aa, Artificial Sequence
  Description of Artificial Sequence Synthetic polypeptide
  MISC_FEATURE (1)..(80): This sequence may encompass 1-20 “Gly Gly Ser Gly” repeating units
GGSGGGSGGGSGGGSGGGSGGGSGGGSGGGSGGGSGGGSGGGSGGGSGGGSGGGSGGGSGGGSGGGSGGGSGGGSGGGSG

SEQ ID NO: 154 - protein, 100 aa, Artificial Sequence
  Description of Artificial Sequence Synthetic polypeptide
GGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGG

SEQ ID NO: 155 - protein, 450 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 156 - protein, 100 aa, Artificial Sequence; identical to SEQ ID NO: 154
  Description of Artificial Sequence Synthetic polypeptide
  MISC_FEATURE (1)..(100): This sequence may encompass 1-20 “Gly Gly Ser Gly Gly” repeating units

SEQ ID NO: 157 - protein, 100 aa, Artificial Sequence; identical to SEQ ID NO: 152
  Description of Artificial Sequence Synthetic polypeptide
  MISC_FEATURE (1)..(100): This sequence may encompass 1-20 “Gly Gly Gly Gly Ser” repeating units

SEQ ID NO: 158 - protein, 5 aa, Artificial Sequence
  Description of Artificial Sequence Synthetic peptide
SYAIS

SEQ ID NO: 159 - protein, 16 aa, Artificial Sequence
  Description of Artificial Sequence Synthetic peptide
GDSSIRHAYYYYGMDV

SEQ ID NO: 160 - protein, 7 aa, Artificial Sequence
  Description of Artificial Sequence Synthetic peptide
SSSYYWG

SEQ ID NO: 161 - protein, 11 aa, Artificial Sequence
  Description of Artificial Sequence Synthetic peptide
GSDRFHPYFDY

SEQ ID NO: 162 - protein, 5 aa, Artificial Sequence
  Description of Artificial Sequence Synthetic peptide
SYYMH

SEQ ID NO: 163 - protein, 17 aa, Artificial Sequence
  Description of Artificial Sequence Synthetic peptide
GAPNYGDTTHDYYYMDV

SEQ ID NO: 164 - protein, 5 aa, Artificial Sequence
  Description of Artificial Sequence Synthetic peptide
GYYMH

SEQ ID NO: 165 - protein, 15 aa, Artificial Sequence
  Description of Artificial Sequence Synthetic peptide
DTGEYYDTDDHGMDV

SEQ ID NO: 166 - protein, 5 aa, Artificial Sequence
  Description of Artificial Sequence Synthetic peptide
SYAMS

SEQ ID NO: 167 - protein, 12 aa, Artificial Sequence
  Description of Artificial Sequence Synthetic peptide
DGGYYDSGAGDY

SEQ ID NO: 168 - protein, 5 aa, Artificial Sequence
  Description of Artificial Sequence Synthetic peptide
SYSMN

SEQ ID NO: 169 - protein, 13 aa, Artificial Sequence
  Description of Artificial Sequence Synthetic peptide
GAPMGAAAGWFDP

SEQ ID NO: 170 - protein, 16 aa, Artificial Sequence
  Description of Artificial Sequence Synthetic peptide
EGAGFAYGMDYYYMDV

SEQ ID NO: 171 - protein, 249 aa, Artificial Sequence
  Description of Artificial Sequence Synthetic polypeptide
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGCGTKVEIKRGGGGSGGGGSGGGGSGGGGSQVQLQQSGPELVKPGASLKLSCTASGFNIKDTYIHWVKQRPEQCLEWIGRIYPTNGYTRYDPKFQDKATITADTSSNTAYLQVSRLTSEDTAVYYCSRWGGDGFYAMDYWGQGASVTVSSA

SEQ ID NO: 172 - protein, 15 aa, Artificial Sequence
  Description of Artificial Sequence Synthetic peptide
ARGAPIGAAAGWFDP

SEQ ID NO: 173 - protein, 13 aa, Artificial Sequence
  Description of Artificial Sequence Synthetic peptide
GAPIGAAAGWFDP

SEQ ID NO: 174 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPQGAAAGWFDPWGQGTLVTVSS

SEQ ID NO: 175 - protein, 15 aa, Artificial Sequence
  Description of Artificial Sequence Synthetic peptide
ARGAPQGAAAGWFDP

SEQ ID NO: 176 - protein, 13 aa, Artificial Sequence
  Description of Artificial Sequence Synthetic peptide
GAPQGAAAGWFDP

SEQ ID NO: 177 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPLGAAAGWFDPWGQGTLVTVSS

SEQ ID NO: 178 - protein, 15 aa, Artificial Sequence
  Description of Artificial Sequence Synthetic peptide
ARGAPLGAAAGWFDP

SEQ ID NO: 179 - protein, 13 aa, Artificial Sequence
  Description of Artificial Sequence Synthetic peptide
GAPLGAAAGWFDP

SEQ ID NO: 180 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPFGAAAGWFDPWGQGTLVTVSS

SEQ ID NO: 181 - protein, 15 aa, Artificial Sequence
  Description of Artificial Sequence Synthetic peptide
ARGAPFGAAAGWFDP

SEQ ID NO: 182 - protein, 13 aa, Artificial Sequence
  Description of Artificial Sequence Synthetic peptide
GAPFGAAAGWFDP

SEQ ID NO: 183 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPVGAAAGWFDPWGQGTLVTVSS

SEQ ID NO: 184 - protein, 15 aa, Artificial Sequence
  Description of Artificial Sequence Synthetic peptide
ARGAPVGAAAGWFDP

SEQ ID NO: 185 - protein, 13 aa, Artificial Sequence
  Description of Artificial Sequence Synthetic peptide
GAPVGAAAGWFDP

SEQ ID NO: 186 - protein, 122 aa, Artificial Sequence
  Description of Artificial Sequence Synthetic polypeptide
  MOD_RES (102)..(102): Met, Leu, Ile, Val, Gln, or Phe
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPXGAAAGWFDPWGQGTLVTVSS

SEQ ID NO: 187 - protein, 15 aa, Artificial Sequence
  Description of Artificial Sequence Synthetic peptide
  MOD_RES (6)..(6): Met, Leu, Ile, Val, Gln, or Phe
ARGAPXGAAAGWFDP

SEQ ID NO: 188 - protein, 13 aa, Artificial Sequence
  Description of Artificial Sequence Synthetic peptide
  MOD_RES (4)..(4): Met, Leu, Ile, Val, Gln, or Phe
GAPXGAAAGWFDP

SEQ ID NO: 189 - protein, 248 aa, Artificial Sequence
  Description of Artificial Sequence Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGCGTKVEIKRGGGGSGGGGSGGGGSGGGGSEVQLVESGGGLVQPGGSLRLSCAASGFTFTDYTMDWVRQAPGKCLEWVADVNPNSGGSIYNQRFKGRFTLSVDRSKNTLYLQMNSLRAEDTAVYYCARNLGPSFYFDYWGQGTLVTVSSA

SEQ ID NO: 190 - protein, 476 aa, Artificial Sequence
  Description of Artificial Sequence Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGCGTKVEIKRGGGGSGGGGSGGGGSGGGGSEVQLVESGGGLVQPGGSLRLSCAASGFTFTDYTMDWVRQAPGKCLEWVADVNPNSGGSIYNQRFKGRFTLSVDRSKNTLYLQMNSLRAEDTAVYYCARNLGPSFYFDYWGQGTLVTVSSAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 191 - protein, 476 aa, Artificial Sequence
  Description of Artificial Sequence Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGCGTKVEIKRGGGGSGGGGSGGGGSGGGGSEVQLVESGGGLVQPGGSLRLSCAASGFTFTDYTMDWVRQAPGKCLEWVADVNPNSGGSIYNQRFKGRFTLSVDRSKNTLYLQMNSLRAEDTAVYYCARNLGPSFYFDYWGQGTLVTVSSAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 192 - protein, 477 aa, Artificial Sequence
  Description of Artificial Sequence Synthetic polypeptide
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGCGTKVEIKRGGGGSGGGGSGGGGSGGGGSQVQLQQSGPELVKPGASLKLSCTASGFNIKDTYIHWVKQRPEQCLEWIGRIYPTNGYTRYDPKFQDKATITADTSSNTAYLQVSRLTSEDTAVYYCSRWGGDGFYAMDYWGQGASVTVSSAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 193 - protein, 477 aa, Artificial Sequence
  Description of Artificial Sequence Synthetic polypeptide
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGCGTKVEIKRGGGGSGGGGSGGGGSGGGGSQVQLQQSGPELVKPGASLKLSCTASGFNIKDTYIHWVKQRPEQCLEWIGRIYPTNGYTRYDPKFQDKATITADTSSNTAYLQVSRLTSEDTAVYYCSRWGGDGFYAMDYWGQGASVTVSSAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 194 - protein, 451 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 195 - protein, 120 aa, Artificial Sequence
  Description of Artificial Sequence Synthetic polypeptide
EVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSS

SEQ ID NO: 196 - protein, 107 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIK

SEQ ID NO: 197 - protein, 120 aa, Artificial Sequence
  Description of Artificial Sequence Synthetic polypeptide
EVQLVESGGGLVQPGGSLRLSCAASGFTFTDYTMDWVRQAPGKCLEWVADVNPNSGGSIYNQRFKGRFTLSVDRSKNTLYLQMNSLRAEDTAVYYCARNLGPSFYFDYWGQGTLVTVSSA

SEQ ID NO: 198 - protein, 108 aa, Artificial Sequence
  Description of Artificial Sequence Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGCGTKVEIKR

SEQ ID NO: 199 - protein, 121 aa, Artificial Sequence
  Description of Artificial Sequence Synthetic polypeptide
QVQLQQSGPELVKPGASLKLSCTASGFNIKDTYIHWVKQRPEQCLEWIGRIYPTNGYTRYDPKFQDKATITADTSSNTAYLQVSRLTSEDTAVYYCSRWGGDGFYAMDYWGQGASVTVSSA

SEQ ID NO: 200 - protein, 108 aa, Artificial Sequence
  Description of Artificial Sequence Synthetic polypeptide
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGCGTKVEIKR

SEQ ID NO: 201 - protein, 20 aa, Artificial Sequence
  Description of Artificial Sequence Synthetic peptide
GSGSGSGSGSGSGSGSGSGS

SEQ ID NO: 202 - protein, 30 aa, Artificial Sequence
  Description of Artificial Sequence Synthetic polypeptide
GGSGGSGGSGGSGGSGGSGGSGGSGGSGGS

SEQ ID NO: 203 - protein, 20 aa, Artificial Sequence
  Description of Artificial Sequence Synthetic peptide
GGGGSGGGGSGGGGSGGGGS

SEQ ID NO: 204 - protein, 40 aa, Artificial Sequence
  Description of Artificial Sequence Synthetic polypeptide
  MISC_FEATURE (1)..(40): This sequence may encompass 1-20 “Gly Ser” repeating units
GSGSGSGSGSGSGSGSGSGSGSGSGSGSGSGSGSGSGSGS

SEQ ID NO: 205 - protein, 60 aa, Artificial Sequence
  Description of Artificial Sequence Synthetic polypeptide
  MISC_FEATURE (1)..(60): This sequence may encompass 1-20 “Gly Gly Ser” repeating units
GGSGGSGGSGGSGGSGGSGGSGGSGGSGGSGGSGGSGGSGGSGGSGGSGGSGGSGGSGGS